SEQUENCE LISTING (573 sequences)

SEQ ID NO: 1 - RNA, 23 nt, Homo sapiens
uggaagacuagugauuuuguugu

SEQ ID NO: 2 - RNA, 22 nt, Homo sapiens
ugagguaguagguuguauaguu

SEQ ID NO: 3 - RNA, 22 nt, Homo sapiens
ugagguaguagguugugugguu

SEQ ID NO: 4 - RNA, 22 nt, Homo sapiens
ugagguaguagguuguaugguu

SEQ ID NO: 5 - RNA, 22 nt, Homo sapiens
agagguaguagguugcauaguu

SEQ ID NO: 6 - RNA, 21 nt, Homo sapiens
ugagguaggagguuguauagu

SEQ ID NO: 7 - RNA, 22 nt, Homo sapiens
ugagguaguagauuguauaguu

SEQ ID NO: 8 - RNA, 22 nt, Homo sapiens
ugagguaguagauuauauaguu

SEQ ID NO: 9 - RNA, 22 nt, Homo sapiens
ugagguaguaguuuguacaguu

SEQ ID NO: 10 - RNA, 22 nt, Homo sapiens
cugcgcaagcuacugccuugcu

SEQ ID NO: 11 - RNA, 22 nt, Homo sapiens
uaguggcuugcuuguaggcugu

SEQ ID NO: 12 - DNA, 22 bp, Homo sapiens
tagcagcacataatggtttgtg

SEQ ID NO: 13 - RNA, 22 nt, Homo sapiens
uagcagcacauaaugguuugug

SEQ ID NO: 14 - RNA, 22 nt, Homo sapiens
uagcagcacaucaugguuuaca

SEQ ID NO: 15 - RNA, 22 nt, Homo sapiens
uagcagcacguaaauauuggcg

SEQ ID NO: 16 - RNA, 24 nt, Homo sapiens
ugauggacgugacauucgugaaac

SEQ ID NO: 17 - RNA, 23 nt, Homo sapiens
uaaggugcaucuagugcagauag

SEQ ID NO: 18 - RNA, 23 nt, Homo sapiens
ugugcaaaucuaugcaaaacuga

SEQ ID NO: 19 - RNA, 23 nt, Homo sapiens
ugugcaaauccaugcaaaacuga

SEQ ID NO: 20 - RNA, 22 nt, Homo sapiens
acugcauuaugagcacuuaaag

SEQ ID NO: 21 - RNA, 23 nt, Homo sapiens
caaagugcucauagugcagguag

SEQ ID NO: 22 - RNA, 22 nt, Homo sapiens
uagcuuaucagacugauguuga

SEQ ID NO: 23 - RNA, 22 nt, Homo sapiens
aagcugccaguugaagaacugu

SEQ ID NO: 24 - RNA, 21 nt, Homo sapiens
aucacauugccagggauuucc

SEQ ID NO: 25 - RNA, 21 nt, Homo sapiens
aucacauugccagggauuacc

SEQ ID NO: 26 - RNA, 22 nt, Homo sapiens
uggcucaguucagcaggaacag

SEQ ID NO: 27 - RNA, 22 nt, Homo sapiens
cauugcacuugucucggucuga

SEQ ID NO: 28 - RNA, 22 nt, Homo sapiens
uucaaguaauccaggauaggcu

SEQ ID NO: 29 - RNA, 21 nt, Homo sapiens
uucaaguaauucaggauaggu

SEQ ID NO: 30 - DNA, 22 bp, Homo sapiens
ttcacagtggctaagttccgct

SEQ ID NO: 31 - RNA, 21 nt, Homo sapiens
uucacaguggcuaaguuccgc

SEQ ID NO: 32 - RNA, 21 nt, Homo sapiens
uucacaguggcuaaguucugc

SEQ ID NO: 33 - RNA, 22 nt, Homo sapiens
uagcaccaucugaaaucgguua

SEQ ID NO: 34 - RNA, 23 nt, Homo sapiens
uagcaccauuugaaaucaguguu

SEQ ID NO: 35 - RNA, 22 nt, Homo sapiens
uagcaccauuugaaaucgguua

SEQ ID NO: 36 - RNA, 34 nt, Homo sapiens
aaccuugcuuccagucgaggauguuuacaccaag

SEQ ID NO: 37 - RNA, 22 nt, Homo sapiens
uguaaacauccuacacucagcu

SEQ ID NO: 38 - RNA, 23 nt, Homo sapiens
uguaaacauccuacacucucagc

SEQ ID NO: 39 - RNA, 22 nt, Homo sapiens
uguaaacauccccgacuggaag

SEQ ID NO: 40 - RNA, 22 nt, Homo sapiens
uguaaacauccuugacuggaag

SEQ ID NO: 41 - RNA, 22 nt, Homo sapiens
uggcagugucuuagcugguugu

SEQ ID NO: 42 - RNA, 22 nt, Caenorhabditis elegans
ucaccggguguaaaucagcuug

SEQ ID NO: 43 - RNA, 24 nt, Caenorhabditis elegans
uacccguaaucuucauaauccgag

SEQ ID NO: 44 - RNA, 21 nt, Homo sapiens
uauugcacuugucccggccug

SEQ ID NO: 45 - RNA, 23 nt, Homo sapiens
caaagugcuguucgugcagguag

SEQ ID NO: 46 - RNA, 22 nt, Homo sapiens
ugagguaguaaguuguauuguu

SEQ ID NO: 47 - RNA, 22 nt, Homo sapiens
cacccguagaaccgaccuugcg

SEQ ID NO: 48 - RNA, 22 nt, Homo sapiens
aacccguagauccgaacuugug

SEQ ID NO: 49 - RNA, 21 nt, Homo sapiens
uacaguacugugauaacugaa

SEQ ID NO: 50 - RNA, 23 nt, Homo sapiens
agcagcauuguacagggcuauga

SEQ ID NO: 51 - RNA, 21 nt, Homo sapiens
uaaagugcugacagugcagau

SEQ ID NO: 52 - RNA, 23 nt, Homo sapiens
agcagcauuguacagggcuauca

SEQ ID NO: 53 - RNA, 24 nt, Homo sapiens
ucccugagacccuuuaaccuguga

SEQ ID NO: 54 - RNA, 22 nt, Homo sapiens
ucccugagacccuaacuuguga

SEQ ID NO: 55 - RNA, 22 nt, Homo sapiens
cagugcaauguuaaaagggcau

SEQ ID NO: 56 - RNA, 22 nt, Homo sapiens
cagugcaaugaugaaagggcau

SEQ ID NO: 57 - RNA, 22 nt, Homo sapiens
uaacagucuacagccauggucg

SEQ ID NO: 58 - RNA, 22 nt, Homo sapiens
uuugguccccuucaaccagcug

SEQ ID NO: 59 - RNA, 23 nt, Homo sapiens
uauggcuuuuuauuccuauguga

SEQ ID NO: 60 - RNA, 23 nt, Homo sapiens
acuccauuuguuuugaugaugga

SEQ ID NO: 61 - RNA, 22 nt, Homo sapiens
uaacacugucugguaaagaugg

SEQ ID NO: 62 - RNA, 20 nt, Homo sapiens
cauaaaguagaaagcacuac

SEQ ID NO: 63 - RNA, 21 nt, Homo sapiens
ugagaugaagcacuguagcuc

SEQ ID NO: 64 - RNA, 22 nt, Homo sapiens
ucagugcaucacagaacuuugu

SEQ ID NO: 65 - RNA, 23 nt, Homo sapiens
ucuggcuccgugucuucacuccc

SEQ ID NO: 66 - RNA, 22 nt, Homo sapiens
ucucccaacccuuguaccagug

SEQ ID NO: 67 - RNA, 21 nt, Homo sapiens
ucagugcaugacagaacuugg

SEQ ID NO: 68 - RNA, 22 nt, Homo sapiens
uagguuauccguguugccuucg

SEQ ID NO: 69 - RNA, 23 nt, Homo sapiens
aacauucauugcugucggugggu

SEQ ID NO: 70 - RNA, 24 nt, Homo sapiens
uuuggcaaugguagaacucacacu

SEQ ID NO: 71 - RNA, 23 nt, Homo sapiens
caacggaaucccaaaagcagcug

SEQ ID NO: 72 - RNA, 21 nt, Homo sapiens
uagcagcacagaaauauuggc

SEQ ID NO: 73 - RNA, 22 nt, Homo sapiens
uagguaguuuccuguuguuggg

SEQ ID NO: 74 - RNA, 23 nt, Homo sapiens
cccaguguucagacuaccuguuc

SEQ ID NO: 75 - RNA, 22 nt, Homo sapiens
acaguagucugcacauugguua

SEQ ID NO: 76 - RNA, 23 nt, Homo sapiens
cccaguguuuagacuaucuguuc

SEQ ID NO: 77 - RNA, 22 nt, Homo sapiens
uaacacugucugguaacgaugu

SEQ ID NO: 78 - RNA, 22 nt, Homo sapiens
uaauacugccugguaaugauga

SEQ ID NO: 79 - RNA, 23 nt, Homo sapiens
uaauacugccggguaaugaugga

SEQ ID NO: 80 - RNA, 22 nt, Homo sapiens
gugaaauguuuaggaccacuag

SEQ ID NO: 81 - RNA, 22 nt, Homo sapiens
uccuucauuccaccggagucug

SEQ ID NO: 82 - RNA, 22 nt, Homo sapiens
cugugcgugugacagcggcuga

SEQ ID NO: 83 - RNA, 21 nt, Homo sapiens
uaacagucuccagucacggcc

SEQ ID NO: 84 - RNA, 22 nt, Homo sapiens
acagcaggcacagacaggcagu

SEQ ID NO: 85 - RNA, 23 nt, Homo sapiens
uacugcaucaggaacugauugga

SEQ ID NO: 86 - RNA, 23 nt, Homo sapiens
agcuacauugucugcuggguuuc

SEQ ID NO: 87 - RNA, 21 nt, Homo sapiens
agcuacaucuggcuacugggu

SEQ ID NO: 88 - RNA, 21 nt, Homo sapiens
caagucacuagugguuccguu

SEQ ID NO: 89 - RNA, 23 nt, Caenorhabditis elegans
uuuguacuccgaugccauucaga

SEQ ID NO: 90 - RNA, 21 nt, Homo sapiens
agggcccccccucaauccugu

SEQ ID NO: 91 - RNA, 22 nt, Homo sapiens
ugguuuaccgucccacauacau

SEQ ID NO: 92 - RNA, 23 nt, Homo sapiens
cgcauccccuagggcauuggugu

SEQ ID NO: 93 - RNA, 22 nt, Homo sapiens
cuagguauggucccagggaucc

SEQ ID NO: 94 - RNA, 23 nt, Homo sapiens
ucccuguccuccaggagcucacg

SEQ ID NO: 95 - RNA, 21 nt, Homo sapiens
aggggugcuaucugugauuga

SEQ ID NO: 96 - RNA, 22 nt, Homo sapiens
uuaucagaaucuccagggguac

SEQ ID NO: 97 - RNA, 22 nt, Homo sapiens
uaaugccccuaaaaauccuuau

SEQ ID NO: 98 - DNA, 20 bp, Homo sapiens
agggagatcgaccgtgttat

SEQ ID NO: 99 - RNA, 22 nt, Homo sapiens
uuuguucguucggcucgcguga

SEQ ID NO: 100 - RNA, 22 nt, Homo sapiens
gaaguuguucgugguggauucg

SEQ ID NO: 101 - RNA, 23 nt, Homo sapiens
cgaauguugcucggugaaccccu

SEQ ID NO: 102 - RNA, 22 nt, Homo sapiens
cagcagcaauucauguuuugaa

SEQ ID NO: 103 - RNA, 22 nt, Homo sapiens
uaauacugucugguaaaaccgu

SEQ ID NO: 104 - RNA, 22 nt, Homo sapiens
uggcaguguauuguuagcuggu

SEQ ID NO: 105 - RNA, 22 nt, Homo sapiens
aggcaguguauuguuagcuggc

SEQ ID NO: 106 - RNA, 22 nt, Homo sapiens
ucaggcucaguccccucccgau

SEQ ID NO: 107 - RNA, 22 nt, Homo sapiens
aaacaaacauggugcacuucuu

SEQ ID NO: 108 - RNA, 23 nt, Homo sapiens
acuuacagacaagagccuugcuc

SEQ ID NO: 109 - RNA, 21 nt, Homo sapiens
uggguuuacguugggagaacu

SEQ ID NO: 110 - RNA, 22 nt, Homo sapiens
guucucccaacguaagcccagc

SEQ ID NO: 111 - RNA, 22 nt, Homo sapiens
uacccauugcauaucggaguug

SEQ ID NO: 112 - RNA, 18 nt, Artificial Sequence
  Primer
agcguguagggauccaaa

SEQ ID NO: 113 - RNA, 24 nt, Artificial Sequence
  Primer
ggccaacguucucaacaauaguga

SEQ ID NO: 114 - RNA, 50 nt, Artificial Sequence
  Primer
rgrururcrargrargrururcrurarcrargrurcrararcrararurc

SEQ ID NO: 115 - DNA, 37 bp, Artificial Sequence
  Primer
caagcagaagacggcatacgattgatggtgcctacag

SEQ ID NO: 116 - DNA, 44 bp, Artificial Sequence
  Primer
aatgatacggcgaccaccgacaggttcagagttctacagtccga

SEQ ID NO: 117 - DNA, 29 bp, Artificial Sequence
  Primer
atcgtrargrgrcrarcrcrurgrarara

SEQ ID NO: 118 - DNA, 15 bp, Artificial Sequence
  Primer
attgatggtgcctac

SEQ ID NO: 119 - DNA, 17 bp, Artificial Sequence
  Primer
attgatggtgcctacag

SEQ ID NO: 120 - DNA, 17 bp, Artificial Sequence
  Primer
atcgtaggcacctgaga

SEQ ID NO: 121 - DNA, 36 bp, Artificial Sequence
  Primer
gcctccctcgcgccatcagatcgtaggcacctgaga

SEQ ID NO: 122 - DNA, 36 bp, Artificial Sequence
  Primer
gccttgccagcccgctcagattgatggtgcctacag

SEQ ID NO: 123 - RNA, 19 nt, Homo sapiens
ggucccaucugggucgcca

SEQ ID NO: 124 - RNA, 22 nt, Homo sapiens
ugugucccauuauuggugauuu

SEQ ID NO: 125 - RNA, 19 nt, Homo sapiens
gcugcgucuuugugcuuuc

SEQ ID NO: 126 - RNA, 19 nt, Homo sapiens
ugucccaucugggucgcca

SEQ ID NO: 127 - RNA, 19 nt, Homo sapiens
gcuccagcccugccggggc

SEQ ID NO: 128 - RNA, 18 nt, Homo sapiens
aucccacuccugacacca

SEQ ID NO: 129 - RNA, 22 nt, Homo sapiens
uucucaaggaggugucguuuau

SEQ ID NO: 130 - RNA, 18 nt, Homo sapiens
gucccuguucgggcgcca

SEQ ID NO: 131 - RNA, 19 nt, Homo sapiens
agucccuucguggucgcca

SEQ ID NO: 132 - RNA, 19 nt, Homo sapiens
agucccaucugggucgcca

SEQ ID NO: 133 - RNA, 22 nt, Homo sapiens
agaggauacccuuuguauguuc

SEQ ID NO: 134 - RNA, 19 nt, Homo sapiens
ugucccuucguggucgcca

SEQ ID NO: 135 - RNA, 20 nt, Homo sapiens
uuuccggcucgcgugggugu

SEQ ID NO: 136 - skipped

SEQ ID NO: 137 - RNA, 22 nt, Homo sapiens
cuuggcaccuagcaagcacuca

SEQ ID NO: 138 - RNA, 18 nt, Homo sapiens
ugguguggucuguuguuu

SEQ ID NO: 139 - RNA, 17 nt, Homo sapiens
ugcauaaggugggucca

SEQ ID NO: 140 - RNA, 17 nt, Homo sapiens
uguugccagucucuagg

SEQ ID NO: 141 - RNA, 22 nt, Homo sapiens
uuagggcccuggcuccaucucc

SEQ ID NO: 142 - RNA, 22 nt, Homo sapiens
cuccguuugccuguuucgcuga

SEQ ID NO: 143 - RNA, 18 nt, Homo sapiens
agggaggaaccaagaugg

SEQ ID NO: 144 - RNA, 19 nt, Homo sapiens
uggggcggagcuuccggag

SEQ ID NO: 145 - RNA, 18 nt, Homo sapiens
aggaaccgcagguucaga

SEQ ID NO: 146 - RNA, 22 nt, Homo sapiens
cuggacugagccgugcuacugg

SEQ ID NO: 147 - RNA, 22 nt, Homo sapiens
uagucccuuccuugaagcgguc

SEQ ID NO: 148 - RNA, 22 nt, Homo sapiens
agcuuccaugacuccugaugga

SEQ ID NO: 149 - RNA, 21 nt, Homo sapiens
uugcagcugccugggagugac

SEQ ID NO: 150 - RNA, 22 nt, Homo sapiens
uucuggaauucugugugaggga

SEQ ID NO: 151 - RNA, 21 nt, Homo sapiens
uguggugcuuauguguguguc

SEQ ID NO: 152 - RNA, 20 nt, Homo sapiens
cguguggugugcgccuguaa

SEQ ID NO: 153 - RNA, 17 nt, Homo sapiens
cgacacaaggguuugaa

SEQ ID NO: 154 - RNA, 17 nt, Homo sapiens
aucccaccgcugccaca

SEQ ID NO: 155 - RNA, 17 nt, Homo sapiens
cucgcugugaugaguga

SEQ ID NO: 156 - RNA, 22 nt, Homo sapiens
aacuagacugugagcuucuaga

SEQ ID NO: 157 - RNA, 22 nt, Homo sapiens
ccaggaauccugcuguggugga

SEQ ID NO: 158 - RNA, 21 nt, Homo sapiens
uguccuugcuguuuggagaua

SEQ ID NO: 159 - RNA, 23 nt, Homo sapiens
cggccccacgcaccaggguaaga

SEQ ID NO: 160 - RNA, 21 nt, Homo sapiens
gguucuuagcauaggaggucu

SEQ ID NO: 161 - RNA, 22 nt, Homo sapiens
uggugcaaaguaauugugguuu

SEQ ID NO: 162 - RNA, 21 nt, Homo sapiens
gcaguaguguagagauugguu

SEQ ID NO: 163 - RNA, 20 nt, Homo sapiens
uuggccccagcuccccgacc

SEQ ID NO: 164 - RNA, 19 nt, Homo sapiens
gagauguuaccuagcguuu

SEQ ID NO: 165 - RNA, 24 nt, Homo sapiens
agcuacaucuggcuacugggucuc

SEQ ID NO: 166 - skipped

SEQ ID NO: 167 - RNA, 21 nt, Homo sapiens
cauuauuacuuuugguacgcg

SEQ ID NO: 168 - RNA, 21 nt, Homo sapiens
uucaaguaauccaggauaggc

SEQ ID NO: 169-170 - skipped (2 entries)

SEQ ID NO: 171 - RNA, 23 nt, Homo sapiens
uguaguguuuccuacuuuaugga

SEQ ID NO: 172 - RNA, 23 nt, Homo sapiens
ucuuugguuaucuagcuguauga

SEQ ID NO: 173 - RNA, 22 nt, Homo sapiens
uucaaguaauucaggauagguu

SEQ ID NO: 174 - RNA, 22 nt, Homo sapiens
caacggaaucccaaaagcagcu

SEQ ID NO: 175 - RNA, 23 nt, Homo sapiens
uaaagugcuuauagugcagguag

SEQ ID NO: 176 - RNA, 22 nt, Homo sapiens
ugagaacugaauuccauggguu

SEQ ID NO: 177-178 - skipped (2 entries)

SEQ ID NO: 179 - RNA, 21 nt, Homo sapiens
uaaagcuagauaaccgaaagu

SEQ ID NO: 180 - RNA, 22 nt, Homo sapiens
uccuguacugagcugccccgag

SEQ ID NO: 181 - RNA, 23 nt, Homo sapiens
caaagaauucuccuuuugggcuu

SEQ ID NO: 182 - RNA, 21 nt, Homo sapiens
ucguaccgugaguaauaaugc

SEQ ID NO: 183-186 - skipped (4 entries)

SEQ ID NO: 187 - RNA, 21 nt, Homo sapiens
ccacaccguaucugacacuuu

SEQ ID NO: 188 - RNA, 22 nt, Homo sapiens
acuagacugaagcuccuugagg

SEQ ID NO: 189-190 - skipped (2 entries)

SEQ ID NO: 191 - RNA, 24 nt, Homo sapiens
caaagugcuuacagugcagguagu

SEQ ID NO: 192 - RNA, 22 nt, Homo sapiens
uguaaacauccucgacuggaag

SEQ ID NO: 193 - RNA, 21 nt, Homo sapiens
agugguuuuacccuaugguag

SEQ ID NO: 194 - RNA, 24 nt, Homo sapiens
ucucacacagaaaucgcacccguc

SEQ ID NO: 195 - RNA, 23 nt, Homo sapiens
aaugacacgaucacucccguuga

SEQ ID NO: 196 - skipped

SEQ ID NO: 197 - RNA, 21 nt, Homo sapiens
uauugcacauuacuaaguugc

SEQ ID NO: 198 - skipped

SEQ ID NO: 199 - RNA, 22 nt, Homo sapiens
uuauaauacaaccugauaagug

SEQ ID NO: 200 - skipped

SEQ ID NO: 201 - RNA, 22 nt, Homo sapiens
ugagaacugaauuccauaggcu

SEQ ID NO: 202 - RNA, 23 nt, Homo sapiens
ucuuggaguaggucauugggugg

SEQ ID NO: 203 - RNA, 22 nt, Homo sapiens
uucaccaccuucuccacccagc

SEQ ID NO: 204 - RNA, 23 nt, Homo sapiens
gcaaagcacacggccugcagaga

SEQ ID NO: 205 - RNA, 22 nt, Homo sapiens
ucggauccgucugagcuuggcu

SEQ ID NO: 206 - RNA, 21 nt, Homo sapiens
ugagguaguaguuuguacagu

SEQ ID NO: 207 - RNA, 23 nt, Homo sapiens
ucccugagacccuuuaaccugug

SEQ ID NO: 208 - RNA, 22 nt, Homo sapiens
cuggcccucucugcccuuccgu

SEQ ID NO: 209 - RNA, 23 nt, Homo sapiens
cagugcaauaguauugucaaagc

SEQ ID NO: 210 - skipped

SEQ ID NO: 211 - RNA, 22 nt, Homo sapiens
accaucgaccguugauuguacc

SEQ ID NO: 212 - RNA, 22 nt, Homo sapiens
aaggagcucacagucuauugag

SEQ ID NO: 213 - RNA, 21 nt, Homo sapiens
aucgggaaugucguguccgcc

SEQ ID NO: 214-216 - skipped (3 entries)

SEQ ID NO: 217 - RNA, 21 nt, Homo sapiens
ugugacugguugaccagaggg

SEQ ID NO: 218-219 - skipped (2 entries)

SEQ ID NO: 220 - RNA, 57 nt, Homo sapiens
gaugaccccagguaacucugagugugucgcugaugccaucaccgcagcgcucugacc

SEQ ID NO: 221 - skipped

SEQ ID NO: 222 - RNA, 22 nt, Homo sapiens
gucauacacggcucuccucucu

SEQ ID NO: 223 - RNA, 21 nt, Homo sapiens
uaguagaccguauagcguacg

SEQ ID NO: 224 - RNA, 23 nt, Homo sapiens
ucaagagcaauaacgaaaaaugu

SEQ ID NO: 225 - skipped

SEQ ID NO: 226 - RNA, 24 nt, Homo sapiens
aacauucauuguugucgguggguu

SEQ ID NO: 227 - RNA, 22 nt, Homo sapiens
gcacauuacacggucgaccucu

SEQ ID NO: 228 - RNA, 21 nt, Homo sapiens
aucauagaggaaaauccacgu

SEQ ID NO: 229 - RNA, 22 nt, Homo sapiens
ucagugcaugacagaacuuggg

SEQ ID NO: 230 - RNA, 22 nt, Homo sapiens
caugccuugaguguaggaccgu

SEQ ID NO: 231 - RNA, 23 nt, Homo sapiens
aaaccguuaccauuacugaguuu

SEQ ID NO: 232 - RNA, 22 nt, Homo sapiens
ccacugccccaggugcugcugg

SEQ ID NO: 233 - RNA, 21 nt, Homo sapiens
aauauaacacagauggccugu

SEQ ID NO: 234 - RNA, 23 nt, Homo sapiens
uccgucucaguuacuuuauagcc

SEQ ID NO: 235 - RNA, 22 nt, Homo sapiens
ucagugcacuacagaacuuugu

SEQ ID NO: 236 - RNA, 22 nt, Homo sapiens
uacaguagucugcacauugguu

SEQ ID NO: 237 - RNA, 22 nt, Homo sapiens
aaucguacagggucauccacuu

SEQ ID NO: 238 - RNA, 23 nt, Homo sapiens
caagucacuagugguuccguuua

SEQ ID NO: 239 - RNA, 22 nt, Homo sapiens
uaaggugcaucuagugcagaua

SEQ ID NO: 240 - RNA, 21 nt, Homo sapiens
uagcaccaucugaaaucgguu

SEQ ID NO: 241 - RNA, 22 nt, Homo sapiens
cuuucagucggauguuuacagc

SEQ ID NO: 242 - RNA, 22 nt, Homo sapiens
uaauacugccggguaaugaugg

SEQ ID NO: 243 - RNA, 21 nt, Homo sapiens
ugucaguuugucaaauacccc

SEQ ID NO: 244 - RNA, 21 nt, Homo sapiens
ucccuguccuccaggagcuca

SEQ ID NO: 245 - RNA, 22 nt, Homo sapiens
uuaaugcuaaucgugauagggg

SEQ ID NO: 246 - RNA, 22 nt, Homo sapiens
aucaugaugggcuccucggugu

SEQ ID NO: 247 - RNA, 21 nt, Homo sapiens
cugaccuaugaauugacagcc

SEQ ID NO: 248-250 - skipped (3 entries)

SEQ ID NO: 251 - RNA, 23 nt, Homo sapiens
aguggggaacccuuccaugagga

SEQ ID NO: 252 - RNA, 60 nt, Homo sapiens
ccuggaugaugauagcaaaugcugacugaacaugaaggucuuaauuagcucuaacugacu

SEQ ID NO: 253 - RNA, 22 nt, Homo sapiens
agcucggucugaggccccucag

SEQ ID NO: 254 - RNA, 21 nt, Homo sapiens
uugguccccuucaaccagcua

SEQ ID NO: 255 - skipped

SEQ ID NO: 256 - RNA, 22 nt, Homo sapiens
uuguacaugguaggcuuucauu

SEQ ID NO: 257-258 - skipped (2 entries)

SEQ ID NO: 259 - RNA, 21 nt, Homo sapiens
ugcugacuccuaguccagggc

SEQ ID NO: 260-262 - skipped (3 entries)

SEQ ID NO: 263 - RNA, 22 nt, Homo sapiens
ugucuuacucccucaggcacau

SEQ ID NO: 264 - RNA, 21 nt, Homo sapiens
uggaauguaaagaaguaugua

SEQ ID NO: 265 - skipped

SEQ ID NO: 266 - RNA, 23 nt, Homo sapiens
gugccuacugagcugauaucagu

SEQ ID NO: 267 - RNA, 22 nt, Homo sapiens
uacaguacugugauaacugaag

SEQ ID NO: 268 - RNA, 24 nt, Homo sapiens
ugaaacauacacgggaaaccucuu

SEQ ID NO: 269 - RNA, 22 nt, Homo sapiens
agaggcuggccgugaugaauuc

SEQ ID NO: 270 - RNA, 23 nt, Homo sapiens
uauggcacugguagaauucacug

SEQ ID NO: 271 - RNA, 20 nt, Homo sapiens
ccucugggcccuuccuccag

SEQ ID NO: 272 - RNA, 20 nt, Homo sapiens
guccgcucggcgguggccca

SEQ ID NO: 273 - RNA, 23 nt, Homo sapiens
ggcagguucucacccucucuagg

SEQ ID NO: 274 - RNA, 20 nt, Homo sapiens
uguaaacauccuugacugga

SEQ ID NO: 275 - RNA, 22 nt, Homo sapiens
uggaagacuagugauuuuguug

SEQ ID NO: 276 - skipped

SEQ ID NO: 277 - RNA, 20 nt, Homo sapiens
acugcagugaaggcacuugu

SEQ ID NO: 278 - RNA, 22 nt, Homo sapiens
aacccguagauccgaucuugug

SEQ ID NO: 279 - RNA, 22 nt, Homo sapiens
aacauucauugcugucgguggg

SEQ ID NO: 280 - RNA, 22 nt, Homo sapiens
ugauauguuugauauauuaggu

SEQ ID NO: 281 - skipped

SEQ ID NO: 282 - RNA, 22 nt, Homo sapiens
aacauucaaccugucggugagu

SEQ ID NO: 283-284 - skipped (2 entries)

SEQ ID NO: 285 - RNA, 22 nt, Homo sapiens
aaagugcuguucgugcagguag

SEQ ID NO: 286 - RNA, 23 nt, Homo sapiens
uacccuguagauccgaauuugug

SEQ ID NO: 287 - RNA, 22 nt, Homo sapiens
uacccuguagaaccgaauuugu

SEQ ID NO: 288 - RNA, 22 nt, Homo sapiens
uaaggugcaucuagugcaguua

SEQ ID NO: 289 - RNA, 22 nt, Homo sapiens
cuuucagucggauguuugcagc

SEQ ID NO: 290 - RNA, 21 nt, Homo sapiens
ggcaagaugcuggcauagcug

SEQ ID NO: 291 - RNA, 21 nt, Homo sapiens
gccccugggccuauccuagaa

SEQ ID NO: 292 - RNA, 23 nt, Homo sapiens
uggcagugucuuagcugguuguu

SEQ ID NO: 293 - RNA, 23 nt, Homo sapiens
uaggcagugucauuagcugauug

SEQ ID NO: 294 - RNA, 23 nt, Homo sapiens
aggcaguguaguuagcugauugc

SEQ ID NO: 295 - RNA, 22 nt, Homo sapiens
uucaacggguauuuauugagca

SEQ ID NO: 296 - RNA, 22 nt, Homo sapiens
uuuggcacuagcacauuuuugc

SEQ ID NO: 297 - skipped

SEQ ID NO: 298 - RNA, 20 nt, Homo sapiens
ucaaaugcucagacuccugu

SEQ ID NO: 299 - RNA, 23 nt, Homo sapiens
uggagugugacaaugguguuugu

SEQ ID NO: 300 - RNA, 22 nt, Homo sapiens
uuaaggcacgcggugaaugcca

SEQ ID NO: 301 - skipped

SEQ ID NO: 302 - RNA, 22 nt, Homo sapiens
ucacagugaaccggucucuuuc

SEQ ID NO: 303 - RNA, 21 nt, Homo sapiens
cuuuuugcggucugggcuugc

SEQ ID NO: 304 - skipped

SEQ ID NO: 305 - RNA, 22 nt, Homo sapiens
uugguccccuucaaccagcugu

SEQ ID NO: 306 - skipped

SEQ ID NO: 307 - RNA, 22 nt, Homo sapiens
uauggcuuuucauuccuaugug

SEQ ID NO: 308 - RNA, 22 nt, Homo sapiens
uauugcuuaagaauacgcguag

SEQ ID NO: 309 - RNA, 18 nt, Homo sapiens
ucuacagugcacgugucu

SEQ ID NO: 310 - skipped

SEQ ID NO: 311 - RNA, 22 nt, Homo sapiens
ugagaugaagcacuguagcuca

SEQ ID NO: 312 - RNA, 20 nt, Homo sapiens
guguguggaaaugcuucugc

SEQ ID NO: 313 - RNA, 22 nt, Homo sapiens
ucuggcuccgugucuucacucc

SEQ ID NO: 314 - RNA, 20 nt, Homo sapiens
uugcauagucacaaaaguga

SEQ ID NO: 315 - RNA, 22 nt, Homo sapiens
uuuggcaaugguagaacucaca

SEQ ID NO: 316 - RNA, 21 nt, Homo sapiens
ugguucuagacuugccaacua

SEQ ID NO: 317 - RNA, 22 nt, Homo sapiens
uggacggagaacugauaagggu

SEQ ID NO: 318 - RNA, 18 nt, Homo sapiens
uggagagaaaggcaguuc

SEQ ID NO: 319 - RNA, 21 nt, Homo sapiens
ucgugucuuguguugcagccg

SEQ ID NO: 320 - RNA, 21 nt, Homo sapiens
aacuggccuacaaagucccag

SEQ ID NO: 321 - RNA, 24 nt, Homo sapiens
aacuggcccucaaagucccgcuuu

SEQ ID NO: 322 - RNA, 22 nt, Homo sapiens
uguaacagcaacuccaugugga

SEQ ID NO: 323 - RNA, 21 nt, Homo sapiens
uagguaguuucauguuguugg

SEQ ID NO: 324 - RNA, 19 nt, Homo sapiens
gguccagaggggagauagg

SEQ ID NO: 325 - skipped

SEQ ID NO: 326 - RNA, 22 nt, Homo sapiens
caucuuaccggacagugcugga

SEQ ID NO: 327 - RNA, 23 nt, Homo sapiens
uaauacugccugguaaugaugac

SEQ ID NO: 328 - RNA, 22 nt, Homo sapiens
agagguauagggcaugggaaaa

SEQ ID NO: 329 - RNA, 22 nt, Homo sapiens
uuuccuaugcauauacuucuuu

SEQ ID NO: 330 - skipped

SEQ ID NO: 331 - RNA, 22 nt, Homo sapiens
uucccuuugucauccuaugccu

SEQ ID NO: 332 - skipped

SEQ ID NO: 333 - RNA, 22 nt, Homo sapiens
uggaauguaaggaagugugugg

SEQ ID NO: 334 - RNA, 22 nt, Homo sapiens
auaagacgagcaaaaagcuugu

SEQ ID NO: 335 - skipped

SEQ ID NO: 336 - RNA, 22 nt, Homo sapiens
uucccuuugucauccuucgccu

SEQ ID NO: 337 - RNA, 21 nt, Homo sapiens
acagcaggcacagacaggcag

SEQ ID NO: 338 - RNA, 21 nt, Homo sapiens
augaccuaugaauugacagac

SEQ ID NO: 339 - RNA, 21 nt, Homo sapiens
uaaucucagcuggcaacugug

SEQ ID NO: 340 - RNA, 24 nt, Homo sapiens
uacugcaucaggaacugauuggau

SEQ ID NO: 341 - RNA, 21 nt, Homo sapiens
uugugcuugaucuaaccaugu

SEQ ID NO: 342 - RNA, 21 nt, Homo sapiens
ugauuguccaaacgcaauucu

SEQ ID NO: 343-344 - skipped (2 entries)

SEQ ID NO: 345 - RNA, 22 nt, Homo sapiens
uaugugggaugguaaaccgcuu

SEQ ID NO: 346 - skipped

SEQ ID NO: 347 - RNA, 23 nt, Homo sapiens
uaagugcuuccauguuuugguga

SEQ ID NO: 348 - RNA, 22 nt, Homo sapiens
uaaacguggauguacuugcuuu

SEQ ID NO: 349 - RNA, 23 nt, Homo sapiens
uaagugcuuccauguuuuaguag

SEQ ID NO: 350 - RNA, 23 nt, Homo sapiens
acuuuaacauggaagugcuuucu

SEQ ID NO: 351 - RNA, 23 nt, Homo sapiens
uaagugcuuccauguuucagugg

SEQ ID NO: 352 - RNA, 22 nt, Homo sapiens
uuuaacauggggguaccugcug

SEQ ID NO: 353 - RNA, 23 nt, Homo sapiens
uaagugcuuccauguuugagugu

SEQ ID NO: 354 - RNA, 23 nt, Homo sapiens
aaaagcuggguugagagggcgaa

SEQ ID NO: 355 - skipped

SEQ ID NO: 356 - RNA, 23 nt, Homo sapiens
ccuaguagguguccaguaagugu

SEQ ID NO: 357 - RNA, 22 nt, Homo sapiens
aacacaccugguuaaccucuuu

SEQ ID NO: 358 - RNA, 23 nt, Homo sapiens
uccagcuccuauaugaugccuuu

SEQ ID NO: 359 - RNA, 23 nt, Homo sapiens
uccagcaucagugauuuuguuga

SEQ ID NO: 360 - RNA, 22 nt, Homo sapiens
cggguggaucacgaugcaauuu

SEQ ID NO: 361 - skipped

SEQ ID NO: 362 - RNA, 22 nt, Homo sapiens
aauugcacuuuagcaaugguga

SEQ ID NO: 363 - RNA, 22 nt, Homo sapiens
acauagaggaaauuccacguuu

SEQ ID NO: 364 - RNA, 21 nt, Homo sapiens
aauaauacaugguugaucuuu

SEQ ID NO: 365 - RNA, 22 nt, Homo sapiens
agaucgaccguguuauauucgc

SEQ ID NO: 366 - RNA, 21 nt, Homo sapiens
gugccgccaucuuuugagugu

SEQ ID NO: 367 - RNA, 23 nt, Homo sapiens
aaagugcugcgacauuugagcgu

SEQ ID NO: 368 - RNA, 23 nt, Homo sapiens
gaagugcuucgauuuuggggugu

SEQ ID NO: 369 - RNA, 22 nt, Homo sapiens
acucaaaaugggggcgcuuucc

SEQ ID NO: 370 - RNA, 21 nt, Homo sapiens
gguagauucuccuucuaugag

SEQ ID NO: 371 - RNA, 22 nt, Homo sapiens
aucauagaggaaaauccauguu

SEQ ID NO: 372 - RNA, 22 nt, Homo sapiens
uauguaauaugguccacaucuu

SEQ ID NO: 373 - RNA, 22 nt, Homo sapiens
ugguugaccauagaacaugcgc

SEQ ID NO: 374 - RNA, 22 nt, Homo sapiens
uauacaagggcaagcucucugu

SEQ ID NO: 375 - RNA, 22 nt, Homo sapiens
agguuacccgagcaacuuugca

SEQ ID NO: 376 - RNA, 23 nt, Homo sapiens
acuucaccugguccacuagccgu

SEQ ID NO: 377 - RNA, 22 nt, Homo sapiens
cuggacuuagggucagaaggcc

SEQ ID NO: 378 - RNA, 22 nt, Homo sapiens
cuggacuuggagucagaaggcc

SEQ ID NO: 379 - skipped

SEQ ID NO: 380 - RNA, 21 nt, Homo sapiens
cuggauggcuccuccaugucu

SEQ ID NO: 381 - RNA, 22 nt, Homo sapiens
uugcauauguaggaugucccau

SEQ ID NO: 382-383 - skipped (2 entries)

SEQ ID NO: 384 - RNA, 22 nt, Homo sapiens
uuuuugcgauguguuccuaaua

SEQ ID NO: 385 - RNA, 22 nt, Homo sapiens
uguuugcagaggaaacugagac

SEQ ID NO: 386 - RNA, 21 nt, Homo sapiens
ucagucucaucugcaaagaag

SEQ ID NO: 387 - RNA, 22 nt, Homo sapiens
gagguuguccguggugaguucg

SEQ ID NO: 388 - RNA, 21 nt, Homo sapiens
cccagauaauggcacucucaa

SEQ ID NO: 389 - RNA, 23 nt, Homo sapiens
agugacaucacauauacggcagc

SEQ ID NO: 390 - RNA, 22 nt, Homo sapiens
caaccuggaggacuccaugcug

SEQ ID NO: 391 - RNA, 23 nt, Homo sapiens
aggaccugcgggacaagauucuu

SEQ ID NO: 392 - RNA, 17 nt, Homo sapiens
auuacauggccaaucuc

SEQ ID NO: 393 - RNA, 21 nt, Homo sapiens
cagcagcacacugugguuugu

SEQ ID NO: 394 - RNA, 22 nt, Homo sapiens
augcaccugggcaaggauucug

SEQ ID NO: 395 - RNA, 22 nt, Homo sapiens
aauccuuugucccugggugaga

SEQ ID NO: 396 - RNA, 21 nt, Homo sapiens
auccuugcuaucugggugcua

SEQ ID NO: 397 - RNA, 23 nt, Homo sapiens
uagcagcgggaacaguucugcag

SEQ ID NO: 398 - RNA, 21 nt, Homo sapiens
agacccuggucugcacucuau

SEQ ID NO: 399 - RNA, 22 nt, Homo sapiens
gucaacacuugcugguuuccuc

SEQ ID NO: 400 - RNA, 21 nt, Homo sapiens
uaaggcacccuucugaguaga

SEQ ID NO: 401 - RNA, 21 nt, Homo sapiens
uuuugcaccuuuuggagugaa

SEQ ID NO: 402 - RNA, 23 nt, Homo sapiens
ugauuguagccuuuuggaguaga

SEQ ID NO: 403 - RNA, 23 nt, Homo sapiens
ugauugguacgucuguggguaga

SEQ ID NO: 404 - RNA, 23 nt, Homo sapiens
uacucaggagaguggcaaucaca

SEQ ID NO: 405 - RNA, 21 nt, Homo sapiens
gugucuuuugcucugcaguca

SEQ ID NO: 406 - RNA, 22 nt, Homo sapiens
aagugcugucauagcugagguc

SEQ ID NO: 407 - RNA, 23 nt, Homo sapiens
cacucagccuugagggcacuuuc

SEQ ID NO: 408 - RNA, 22 nt, Homo sapiens
uucacagggaggugucauuuau

SEQ ID NO: 409 - RNA, 20 nt, Homo sapiens
auugacacuucugugaguag

SEQ ID NO: 410 - RNA, 21 nt, Homo sapiens
gagugccuucuuuuggagcgu

SEQ ID NO: 411 - RNA, 24 nt, Homo sapiens
uucuccaaaagaaagcacuuucug

SEQ ID NO: 412 - RNA, 23 nt, Homo sapiens
caucuggagguaagaagcacuuu

SEQ ID NO: 413 - RNA, 22 nt, Homo sapiens
ccucuagauggaagcacugucu

SEQ ID NO: 414 - RNA, 23 nt, Homo sapiens
aucgugcaucccuuuagaguguu

SEQ ID NO: 415 - RNA, 22 nt, Homo sapiens
ucgugcaucccuuuagaguguu

SEQ ID NO: 416 - RNA, 22 nt, Homo sapiens
aucgugcauccuuuuagagugu

SEQ ID NO: 417 - RNA, 21 nt, Homo sapiens
aaagcgcuucccuuugcugga

SEQ ID NO: 418 - RNA, 22 nt, Homo sapiens
caaagcgcuccccuuuagaggu

SEQ ID NO: 419 - RNA, 22 nt, Homo sapiens
caaagcgcuucucuuuagagug

SEQ ID NO: 420 - RNA, 23 nt, Homo sapiens
ucucuggagggaagcacuuucug

SEQ ID NO: 421 - RNA, 21 nt, Homo sapiens
caaagcgcuucccuuuggagc

SEQ ID NO: 422 - RNA, 22 nt, Homo sapiens
aaagcgcuucccuucagagugu

SEQ ID NO: 423 - RNA, 21 nt, Homo sapiens
aaagcgcuucucuuuagagga

SEQ ID NO: 424 - RNA, 23 nt, Homo sapiens
aaagugcauccuuuuagagguuu

SEQ ID NO: 425 - RNA, 22 nt, Homo sapiens
aaagugcaucuuuuuagaggau

SEQ ID NO: 426 - RNA, 23 nt, Homo sapiens
caaagugccucccuuuagagugu

SEQ ID NO: 427 - RNA, 22 nt, Homo sapiens
aaagugccuccuuuuagagugu

SEQ ID NO: 428 - RNA, 22 nt, Homo sapiens
aaagugcuucccuuuggacugu

SEQ ID NO: 429 - RNA, 21 nt, Homo sapiens
aaagugcuuccuuuuagaggg

SEQ ID NO: 430 - RNA, 23 nt, Homo sapiens
aaagugcuuccuuuuagaggguu

SEQ ID NO: 431 - RNA, 22 nt, Homo sapiens
agaucagaaggugauuguggcu

SEQ ID NO: 432 - RNA, 21 nt, Homo sapiens
agagguaguagguugcauagu

SEQ ID NO: 433 - RNA, 20 nt, Homo sapiens
uagcaccauuugaaaucggu

SEQ ID NO: 434 - RNA, 24 nt, Homo sapiens
aauccuuggaaccuaggugugagu

SEQ ID NO: 435 - RNA, 22 nt, Homo sapiens
cuccugacuccagguccugugu

SEQ ID NO: 436 - RNA, 19 nt, Homo sapiens
gugcauuguaguugcauug

SEQ ID NO: 437 - RNA, 24 nt, Homo sapiens
guccaguuuucccaggaaucccuu

SEQ ID NO: 438 - RNA, 19 nt, Homo sapiens
ugguagacuauggaacgua

SEQ ID NO: 439 - RNA, 21 nt, Homo sapiens
uagguaguuuccuguuguugg

SEQ ID NO: 440 - RNA, 23 nt, Homo sapiens
aaagugcuucucuuugguggguu

SEQ ID NO: 441 - RNA, 21 nt, Homo sapiens
aaagugcuuccuuuuugaggg

SEQ ID NO: 442 - RNA, 22 nt, Homo sapiens
aagugcuuccuuuuagaggguu

SEQ ID NO: 443 - RNA, 24 nt, Homo sapiens
acaaagugcuucccuuuagagugu

SEQ ID NO: 444 - RNA, 22 nt, Homo sapiens
acaaagugcuucccuuuagagu

SEQ ID NO: 445 - RNA, 22 nt, Homo sapiens
aacgcacuucccuuuagagugu

SEQ ID NO: 446 - RNA, 23 nt, Homo sapiens
aaaaugguucccuuuagaguguu

SEQ ID NO: 447 - RNA, 21 nt, Homo sapiens
aacgcgcuucccuauagaggg

SEQ ID NO: 448 - RNA, 21 nt, Homo sapiens
gaaggcgcuucccuuuggagu

SEQ ID NO: 449 - RNA, 21 nt, Homo sapiens
aaagugcuuccuuuuagaggc

SEQ ID NO: 450 - RNA, 22 nt, Homo sapiens
ucggggaucaucaugucacgag

SEQ ID NO: 451 - RNA, 20 nt, Homo sapiens
auucugcauuuuuagcaagu

SEQ ID NO: 452 - RNA, 22 nt, Homo sapiens
aucagcaaacauuuauugugug

SEQ ID NO: 453 - RNA, 22 nt, Homo sapiens
caaaacuggcaauuacuuuugc

SEQ ID NO: 454 - RNA, 22 nt, Homo sapiens
caagaaccucaguugcuuuugu

SEQ ID NO: 455 - RNA, 22 nt, Homo sapiens
caaaaaucucaauuacuuuugc

SEQ ID NO: 456 - RNA, 22 nt, Homo sapiens
caaaaaccacaguuucuuuugc

SEQ ID NO: 457 - RNA, 21 nt, Homo sapiens
ugacaacuauggaugagcucu

SEQ ID NO: 458 - RNA, 21 nt, Homo sapiens
gcgacccacucuugguuucca

SEQ ID NO: 459-488 - skipped (30 entries)

SEQ ID NO: 489 - RNA, 21 nt, Homo sapiens
gcgacccauacuugguuucag

SEQ ID NO: 490 - RNA, 21 nt, Homo sapiens
aacaggugacugguuagacaa

SEQ ID NO: 491 - RNA, 21 nt, Homo sapiens
aaaacggugagauuuuguuuu

SEQ ID NO: 492 - RNA, 21 nt, Homo sapiens
gcuaguccugacucagccagu

SEQ ID NO: 493 - RNA, 21 nt, Homo sapiens
aggguaagcugaaccucugau

SEQ ID NO: 494 - RNA, 20 nt, Homo sapiens
gaugagcucauuguaauaug

SEQ ID NO: 495 - RNA, 19 nt, Homo sapiens
ugagcugcuguaccaaaau

SEQ ID NO: 496 - RNA, 20 nt, Homo sapiens
aaaguagcuguaccauuugc

SEQ ID NO: 497 - RNA, 19 nt, Homo sapiens
agguugacauacguuuccc

SEQ ID NO: 498 - RNA, 19 nt, Homo sapiens
aggcacggugucagcaggc

SEQ ID NO: 499 - RNA, 22 nt, Homo sapiens
ggcuggcucgcgaugucuguuu

SEQ ID NO: 500 - RNA, 19 nt, Homo sapiens
gggcgccugugaucccaac

SEQ ID NO: 501 - RNA, 21 nt, Homo sapiens
aguuaaugaauccuggaaagu

SEQ ID NO: 502 - RNA, 22 nt, Homo sapiens
gaaaacagcaauuaccuuugca

SEQ ID NO: 503 - RNA, 19 nt, Homo sapiens
gagccaguuggacaggagc

SEQ ID NO: 504 - RNA, 23 nt, Homo sapiens
auucuaauuucuccacgucuuug

SEQ ID NO: 505 - RNA, 21 nt, Homo sapiens
cuucuugugcucuaggauugu

SEQ ID NO: 506 - RNA, 23 nt, Homo sapiens
auucauuugguauaaaccgcgau

SEQ ID NO: 507 - RNA, 22 nt, Homo sapiens
uugagaaugaugaaucauuagg

SEQ ID NO: 508 - RNA, 19 nt, Homo sapiens
ugggcguaucuguaugcua

SEQ ID NO: 509 - RNA, 22 nt, Homo sapiens
uaugcauuguauuuuuaggucc

SEQ ID NO: 510 - RNA, 21 nt, Homo sapiens
uuuccauaggugaugagucac

SEQ ID NO: 511 - RNA, 21 nt, Homo sapiens
uuggccacaauggguuagaac

SEQ ID NO: 512 - RNA, 24 nt, Homo sapiens
ucagaacaaaugccgguucccaga

SEQ ID NO: 513 - RNA, 20 nt, Homo sapiens
agaccauggguucucauugu

SEQ ID NO: 514 - RNA, 25 nt, Homo sapiens
aggcaccagccaggcauugcucagc

SEQ ID NO: 515 - RNA, 25 nt, Homo sapiens
cccaucugggguggccugugacuuu

SEQ ID NO: 516 - RNA, 21 nt, Homo sapiens
aagccugcccggcuccucggg

SEQ ID NO: 517 - RNA, 22 nt, Homo sapiens
ugugucacucgaugaccacugu

SEQ ID NO: 518 - RNA, 20 nt, Homo sapiens
guugugucaguuuaucaaac

SEQ ID NO: 519 - skipped

SEQ ID NO: 520 - RNA, 22 nt, Homo sapiens
uggucuaggauuguuggaggag

SEQ ID NO: 521 - RNA, 22 nt, Homo sapiens
cacacacugcaauuacuuuugc

SEQ ID NO: 522 - RNA, 21 nt, Homo sapiens
aaacuacugaaaaucaaagau

SEQ ID NO: 523 - RNA, 21 nt, Homo sapiens
guucaaauccagaucuauaac

SEQ ID NO: 524 - RNA, 25 nt, Homo sapiens
aggggugguguugggacagcuccgu

SEQ ID NO: 525 - RNA, 21 nt, Homo sapiens
aggguguuuccucucaucucu

SEQ ID NO: 526 - RNA, 20 nt, Homo sapiens
aggaauguuccuucuuugcc

SEQ ID NO: 527 - RNA, 23 nt, Homo sapiens
gaacgccuguucuugccaggugg

SEQ ID NO: 528 - RNA, 21 nt, Homo sapiens
uccgagccugggucucccucu

SEQ ID NO: 529 - RNA, 22 nt, Homo sapiens
acucaaaacccuucagugacuu

SEQ ID NO: 530 - RNA, 22 nt, Homo sapiens
agacuucccauuugaagguggc

SEQ ID NO: 531 - RNA, 23 nt, Homo sapiens
aaacucuacuuguccuucugagu

SEQ ID NO: 532 - RNA, 21 nt, Homo sapiens
acagucugcugagguuggagc

SEQ ID NO: 533 - RNA, 22 nt, Homo sapiens
uaguaccaguaccuuguguuca

SEQ ID NO: 534 - RNA, 19 nt, Homo sapiens
agcugucugaaaaugucuu

SEQ ID NO: 535 - RNA, 22 nt, Homo sapiens
gugagucucuaagaaaagagga

SEQ ID NO: 536 - skipped

SEQ ID NO: 537 - RNA, 22 nt, Homo sapiens
aguauucuguaccagggaaggu

SEQ ID NO: 538 - RNA, 21 nt, Homo sapiens
agaccuggcccagaccucagc

SEQ ID NO: 539 - RNA, 23 nt, Homo sapiens
cuaauaguaucuaccacaauaaa

SEQ ID NO: 540 - RNA, 23 nt, Homo sapiens
aucgcugcgguugcgagcgcugu

SEQ ID NO: 541 - RNA, 22 nt, Homo sapiens
gucccucuccaaaugugucuug

SEQ ID NO: 542 - RNA, 19 nt, Homo sapiens
aguguggcuuucuuagagc

SEQ ID NO: 543 - RNA, 19 nt, Homo sapiens
ucuaggcugguacugcuga

SEQ ID NO: 544 - RNA, 19 nt, Homo sapiens
aagcagcugccucugaggc

SEQ ID NO: 545 - RNA, 21 nt, Homo sapiens
guggcugcacucacuuccuuc

SEQ ID NO: 546 - RNA, 22 nt, Homo sapiens
aaaccuguguuguucaagaguc

SEQ ID NO: 547 - RNA, 21 nt, Homo sapiens
aggaggcagcgcucucaggac

SEQ ID NO: 548 - RNA, 22 nt, Homo sapiens
uuuaggauaagcuugacuuuug

SEQ ID NO: 549 - RNA, 23 nt, Homo sapiens
aauggcgccacuaggguugugca

SEQ ID NO: 550 - RNA, 21 nt, Homo sapiens
uugaaacaaucucuacugaac

SEQ ID NO: 551 - RNA, 22 nt, Homo sapiens
uggugggccgcagaacaugugc

SEQ ID NO: 552 - RNA, 21 nt, Homo sapiens
aauauuauacagucaaccucu

SEQ ID NO: 553 - RNA, 25 nt, Homo sapiens
ggcggagggaaguagguccguuggu

SEQ ID NO: 554 - RNA, 22 nt, Homo sapiens
cuugguucagggagggucccca

SEQ ID NO: 555 - RNA, 24 nt, Homo sapiens
ugccugggucucuggccugcgcgu

SEQ ID NO: 556 - RNA, 21 nt, Homo sapiens
ucccacguuguggcccagcag

SEQ ID NO: 557 - RNA, 42 nt, Homo sapiens
cgcaaggaugacacgcaaauucgugaagcguuccauauuuuu

SEQ ID NO: 558 - RNA, 52 nt, Homo sapiens
rgrururcrargrargrururcrurarcrargrurcrcrgrarcrgrarurc

SEQ ID NO: 559 - skipped

SEQ ID NO: 560 - RNA, 22 nt, Homo sapiens
guaaggcacccuucugaguaga

SEQ ID NO: 561 - RNA, 23 nt, Homo sapiens
uguaaggcacccuucugaguaga

SEQ ID NO: 562 - RNA, 21 nt, Homo sapiens
guaaggcacccuucugaguag

SEQ ID NO: 563 - RNA, 22 nt, Homo sapiens
uguaaggcacccuucugaguag

SEQ ID NO: 564 - RNA, 23 nt, Homo sapiens
ugauugacacuucugugaguaga

SEQ ID NO: 565 - RNA, 20 nt, Homo sapiens
ugauugacacuucugugagu

SEQ ID NO: 566 - RNA, 22 nt, Homo sapiens
ugauugacacuucugugaguag

SEQ ID NO: 567 - RNA, 23 nt, Homo sapiens
uugauugacacuucugugaguag

SEQ ID NO: 568 - RNA, 124 nt, Homo sapiens
gccaccaccaucagccauacuauguguagugccuuauucaggaagguguuacuuaauagauuaauauuuguaaggcacccuucugaguagaguaaugugcaacauggacaacauuugugguggc

SEQ ID NO: 569 - RNA, 124 nt, Homo sapiens; identical to SEQ ID NO: 568

SEQ ID NO: 570 - RNA, 124 nt, Homo sapiens; identical to SEQ ID NO: 568

SEQ ID NO: 571 - RNA, 98 nt, Homo sapiens
aacauguugucugugguacccuacucuggagagugacaaucauguauaauuaaauuugauugacacuucugugaguagaguaacgcaugacacguacg

SEQ ID NO: 572 - RNA, 100 nt, Homo sapiens
aacauguugucugugguacccuacucuggagagagugacaaucauguauaauuaaauuugauugacacuucugugaguagaguaacgcaugacacguacg

SEQ ID NO: 573 - RNA, 98 nt, Homo sapiens; identical to SEQ ID NO: 571

(19) United States
(12) Patent Application Publication
(10) Pub. No.: US 2011/0160290 A1
(43) Pub. Date: Jun. 30, 2011

(54) USE OF EXTRACELLULAR RNA TO MEASURE DISEASE

(76) Inventor: Muneesh Tewari, Seattle, WA (US)

(21) Appl. No.: 12/993,828

(22) PCT Filed: May 21, 2009
(86) PCT No.: PCT/US2009/044906

§ 371 Date: Feb. 18, 2011

Related U.S. Application Data
(60) Provisional application No. 61/054,984, filed on May 21, 2008.
(60) Provisional application No. 61/055,970, filed on May 24, 2008.
(60) Provisional application No. 61/057,818, filed on May 30, 2008.

(51) Int. Cl. A61K 31/7088; G01N 33/50; C12Q 1/68; C40B 30/04; A61P 35/00

(52) U.S. Cl. 514/44 R; 436/94; 435/6.12; 506/9

(57) ABSTRACT

Stable, extracellular microRNAs and methods for isolating and identifying such microRNAs from a body fluid are provided. The extracellular microRNAs isolated from a bodily fluid of a subject can be used to measure disease and provide sensitive, efficient, and non invasive methods for the detection of disease, including cancer. The extracellular microRNAs can be used to develop new therapeutics for the treatment of disease, including cancer. The examples illustrate diagnosis of prostate and ovarian cancer and differential expression of miR-100, miR-135b, miR-141, miR-148a, miR-200a, miR-200c, miR-210, miR-222, miR-375, miR-425-Sp and miR-429.

This application claims priority to U.S. applications 61/057,818, filed 30 May 2008, 61/055,970, filed 24 May 2008, and 61/054,984, filed 21 May 2008, hereby incorporated by reference in their entirety.

### BACKGROUND

MicroRNAs (miRNAs) are small, noncoding RNAs that influence gene regulatory networks by post-transcriptional regulation of specific messenger RNA (mRNA) targets via specific base-pairing interactions. More than 9,500 miRNA have been identified and are recorded on a miRNA registry maintained by the Sanger Institute and available on its website microRNA@sanger.ac.uk New miRNA sequences continue to be discovered by sequencing small RNA (e.g., 18-25 nucleotides in length) isolated from normal or diseased cells; quite possibly the number of miRNA sequences may be on par with the number of coding mRNA in a mammalian cell.

Over-expression and silencing of specific miRNAs have been described in a number of diseases, including cancer. The miR-17-92 cluster is overexpressed in tumor samples from lymphoma patients, and this overexpression is correlated with amplification of the particular region of chromosome 13 in which the miR cluster is located (He et al., 2005, Nature 435:828-33); miR-342 is commonly suppressed in human colorectal cancer (Grady et al., 2008, Oncogene, Feb. 11 2008, No: 18264139); miR-15 and miR-16 are under-expressed in chronic lymphocytic leukemia as a result of a deletion on chromosome 13 (Calin et al., 2005 Proc. Natl. Acad. Sci. USA 99:15524-29); reduced expression of let-7 miRNA is correlated with poor prognosis in lung cancers (Takamizawa et al., 2004, Cancer Res. 64:3753-56); and let-7 may acts as a tumor suppressor by inhibiting the expression of the RAS oncogene lung tissue (Johnson et al., 2005, Cell 120:635-47).

MiRNAs are also involved in the development and function of the cardiovascular system. For example, specific miRNAs have been implicated in vascular angiogenesis and cardiomyocyte apoptosis, and also in the development of cardiac hypertrophy, arrhythmia, and heart failure, and in numerous other diseases and developmental processes, including schizophrenia, Alzheimer's disease, immune cell development and modulation of both adaptive and innate immunity, stem cell maintenance and pluripotency, nervous system development, endocrine disease, including diabetes, development of the pancreas, Fragile X Syndrome, cutaneous wound healing, cell cycle progression, transplanted tissue rejection, hypoxia, skeletal muscle differentiation. MiRNAs are also expressed by viruses, and target genes of those miRNA have been identified.

Given the important functional role of miRNA in disease, this set of nucleic acid molecules contains candidates for diagnosing and prognosing disease, and monitoring response to therapies in a wide variety of patients and in subjects prior to manifesting disease. This potential utility is severely limited however by current methods, which are limited to extracting RNA from cells. Further, the tissue responsible for many disease conditions is not accessible to biopsy or may not be detectable until a late stage of disease. There remains an unmet need to diagnose disease using miRNA in a readily available biological sample, such as blood, serum or plasma miRNA dysregulation contributes to cancer diagnosis and therapy and the identification of specific diagnostics and therapeutic targets is needed to provide new and useful tools for improved management of disease.

### SUMMARY

In one aspect, the present invention provides a method for the detection, classification, diagnosis, prognosis, or monitoring treatment response, of disease or disorder in a human subject, by determining the amount of a miRNA in a biological sample obtained non-invasively from the human subject. The comparative abundance of one or more miRNA, for example an increased or decreased expression of the miRNA in the biological sample relative to a control sample, is used to indicate detection, classification, diagnosis, prognosis, and/or response to treatment of the disease in the subject. The detection methods preferably include normalizing the amount of miRNA to an internal control, preferably a spiked-in control added to the biological sample.

The biological sample can be blood and fractions thereof, blood serum, blood plasma, urine, excreta, semen, seminal fluid, seminal plasma, prostatic fluid, pre-ejaculatory fluid (Cowper's fluid), pleural effusion, tears, saliva, sputum, sweat, biopsy, ascites, cerebrospinal fluid, amniotic fluid, lymph, marrow, cervical secretions, vaginal secretions, endometrial secretions, gastrointestinal secretions, bronchial secretions, breast secretions, ovarian cyst secretions, hair, and tissue extract samples, and the like.

In one method, a miRNA can be purified as RNA from the biological sample, reverse transcribed from the miRNA to form a miRNA cDNA, and amplifying the cDNA by PCR. The spiked in control is preferably added to the purified miRNA prior to reverse transcription, and preferably is added to the body fluid sample during processing for RNA isolation immediately after denaturation and inactivation of RNases in the body fluid sample. This permits the control to reflect technical variations in RNA isolation from sample to sample.

Amplification can be performed using quantitative polymerase chain reaction analysis, with identity determined on the basis of the specificity of the primers. Identity of a purified miRNA can also be determined, for example, by hybridization techniques, antibody binding, or by sequencing.

Provided are methods comprising: contacting RNA of a subject with at least one nucleic acid probe to measure blood levels of at least one miRNA selected from the group consisting of mir-100, miR-135b, miR-141, miR-148a, miR-200a, miR-200c, miR-210, miR-222, miR-375, miR-425-5p and miR-429.

Another aspect of the inventions are methods comprising: administering to a subject for treatment of prostate cancer an effective therapeutic amount of a drug, wherein said subject is identified by the blood levels of at least one miRNA selected from the group consisting of mir-100, miR-135b, miR-141, miR-148a, miR-200a, miR-200c, miR-210, miR-222, miR-375, miR-42.5-5p and miR-429.

Another aspect of the inventions are methods comprising: providing a blood sample from a subject having prostate cancer; extracting RNA from said sample, and contacting said RNA with at least one nucleic acid probe to measure blood levels of at least one miRNA selected from the group consisting of mir-100, miR-135b, miR-141, miR-148a, miR-200a, miR-200c, miR-210, miR-222, miR-375, miR-425-5p and miR-429, wherein said blood levels indicates a therapy suitable for said subject.

Another aspect of the inventions are methods comprising: providing a blood sample from a subject having prostate cancer; extracting RNA from said sample; contacting said RNA with a microarray comprising a multiplicity of single stranded oligonucleotides to measure blood levels of at least one miRNA selected from the group consisting of mir-100, miR-135b, miR-141, miR-148a, miR-200a, miR-200c, miR-210, miR-222, miR-375, miR-425-5p and miR-429; and transforming said expression levels into a probability value by a computer.

Another aspect of the inventions are methods comprising: measuring blood levels of at least one gene selected from the group consisting of mir-100, miR-135b, miR-141, miR-148a, miR-200a, miR-200c, miR-210, miR-222, miR-375, miR-425-5p and miR-429 from a subject having prostate cancer; transforming said blood levels into a probability value by a computer; repeating step a at one or more time points during treatment of said subject for prostate cancer, wherein a low probability value indicates effective treatment.

The spiked in control generally comprises one or more miRNA species that does not interfere with the detection of miRNA(s), and preferably does not react with known human miRNAs and do not cross-hybridize with oligonucleotide probes designed to detect human miRNAs. In the preferred embodiment, the internal control comprises at least 3 miRNA species. The spiked in control miRNAs can be, for example, non-human miRNAs that do not hybridize to human RNAs, for example, one, two, or three of C. elegans miRNAs cel-miR-39, cel-miR-54, and cel-miR-238.

In one embodiment, identity is determined or confirmed by high throughput sequencing or ultra-high throughput sequencing, and particularly by massively parallel sequencing such as 454® Sequencing and Solexa® sequencing.

In one aspect, normalizing the amount of miRNA comprises multiplying the number of copies of a miRNA in the body fluid sample by a normalization factor to obtain a normalized copy number. The normalization factor equals 1/(2^{ΔCt}) wherein

ΔCt=(median control cycle threshold(Ct))−(average control Ct).

The normalized cycle threshold (Ct) for a miRNA in the body fluid sample is calculated according to the following equations:

Normalized Ct=Raw miRNA Ct−ΔCt, and

ΔCt=(median control Ct)−(average control Ct).

The ratio of expression of the miRNA in the body fluid sample relative to the control is calculated as 2^{(-ΔΔCt)}; wherein

ΔΔCt=ΔCt_{miRNA}−ΔCt_{control},

ΔCt_{miRNA}=(Raw miRNA Ct)−(average control Ct), and

ΔCt_{control}=(Raw control Ct)−(average control Ct).

In general, a ratio that is greater than or equal to about 1.2 and less than or equal to about 0.8 indicates a positive, i.e., detection, classification, diagnosis, or prognosis of the disease in the individual.

In another aspect, the present invention provides a method for identifying one or more miRNA candidates for detecting and diagnosing a disease or disorder. In one embodiment, the method comprises differential expression profiling of disease versus normal body fluid miRNAs. An expression profile of miRNA is determined for a sample of body fluid obtained from an individual suffering from the disease or disorder and is compared with a miRNA profile of a sample obtained from an individual who is not suffering from the disease or disorder, e.g., “normal”. Spiked-in control miRNA can be used to normalize the data, as described above. Identification of body fluid miRNAs that are differentially expressed in a disease sample compared the normal sample is indicative of a candidate for the disease or disorder.

The present invention provides one or more miRNA for diagnosis of a disease or disorder, for example cancer. In one embodiment, methods for detecting an epithelial cell cancer in an individual are provided. This method comprises analyzing a sample of body fluid from an individual for expression of one or more miRNA. Diagnosis can be based on the level of a miRNA in a given individual relative to a set threshold, or relative to a standard, where expression, absence of expression, and/or relative abundance of one or more miRNA is indicative of epithelial cell cancer, including, but not limited to, prostate cancer and ovarian cancer. miRNAs for use in this method can include, but are not limited to, mir-100, miR-135b, miR-141, miR-148a, miR-200a, miR-200c, miR-210, miR-222, miR-375, miR-425-5p and miR-429, or a combination thereof.

Method for detection of a disease or disorder comprising detection of miRNAs in body fluid can further include a detection of other biological components, such as nucleic acids, analytes, and physical and medical history measures, such as age, weight, history of diabetes or heart disease, smoking status, occupational exposures, diet, known genetic markers, and the like.

The present invention provides a method of treating a disease or disorder characterized by differential expression of a miRNA in a body fluid. In those instances where a miRNA is overexpressed, the treatment comprises administering an antagonist of the miRNA, such as an antisense molecule or inactive variant or portion of the miRNA. Where the miRNA is underexpressed, the treatment comprises replenishing the miRNA, for example by inducing expression of endogenous miRNA or delivering an exogenous version of the miRNA.

### BRIEF DESCRIPTION OF THE DRAWINGS

FIG. 1 compares miR-141 in serum of advanced prostate cancer patients versus healthy controls (top left panel), and a ROC plot (top right panel) reflects strong separation between the two groups; serum levels of non-candidate miRNAs miR-16, miR-19b, and miR-24 (bottom panels) are shown for comparison.

### DESCRIPTION OF THE SEQUENCES

TABLE 1

| SEQ ID | Name | Type | Species | Sequence |
|---|---|---|---|---|
| 1 | miR-7 | RNA | Human | uggaagacuagugauuuuguugu |
| 2 | miR-let-7a | RNA | Human | ugagguaguagguuguauaguu |
| 3 | miR-let-7b | RNA | Human | ugagguaguagguugugugguu |
| 4 | miR-let-7c | RNA | Human | ugagguaguagguuguaugguu |
| 5 | miR-let-7d | RNA | Human | agagguaguagguugcauaguu |
| 6 | miR-let-7e | RNA | Human | ugagguaggagguuguauagu |
| 7 | miR-let-7f | RNA | Human | ugagguaguagauuguauaguu |
| 8 | miR-let-7f G15A | RNA | Human | ugagguaguagauuauauaguu |
| 9 | miR-let-7g | RNA | Human | ugagguaguaguuuguacaguu |
| 10 | miR-let-7i | RNA | Human | cugcgcaagcuacugccuugcu |
| 11 | miR-9-3p | RNA | Human | uaguggcuugcuuguaggcugu |
| 12 | miR-15 | RNA | Human | tagcagcacataatggtttgtg |
| 13 | miR-15a | RNA | Human | uagcagcacauaaugguuugug |
| 14 | miR-15b | RNA | Human | uagcagcacaucaugguuuaca |
| 15 | miR-16 | RNA | Human | uagcagcacguaaauauuggcg |
| 16 | miR-17-5p | RNA | Human | ugauggacgugacauucgugaaac |
| 17 | miR-18a | RNA | Human | uaaggugcaucuagugcagauag |
| 18 | miR-19a | RNA | Human | ugugcaaaucuaugcaaaacuga |
| 19 | miR-19b | RNA | Human | ugugcaaauccaugcaaaacuga |
| 20 | miR-20a | RNA | Human | acugcauuaugagcacuuaaag |
| 21 | miR-20b | RNA | Human | caaagugcucauagugcagguag |
| 22 | miR-21 | RNA | Human | uagcuuaucagacugauguuga |
| 23 | miR-22 | RNA | Human | aagcugccaguugaagaacugu |
| 24 | miR-23a | RNA | Human | aucacauugccagggauuucc |
| 25 | miR-23b | RNA | Human | aucacauugccagggauuacc |
| 26 | miR-24 | RNA | Human | uggcucaguucagcaggaacag |
| 27 | miR-25 | RNA | Human | cauugcacuugucucggucuga |
| 28 | miR-26a | RNA | Human | uucaaguaauccaggauaggcu |
| 29 | miR-26b | RNA | Human | uucaaguaauucaggauaggu |
| 30 | miR-27 | RNA | Human | ttcacagtggctaagttccgct |
| 31 | miR-27a | RNA | Human | uucacaguggcuaaguuccgc |
| 32 | miR-27b | RNA | Human | uucacaguggcuaaguucugc |
| 33 | miR-29a | RNA | Human | uagcaccaucugaaaucgguua |
| 34 | miR-29b | RNA | Human | uagcaccauuugaaaucaguguu |
| 35 | miR-29c | RNA | Human | uagcaccauuugaaaucgguua |
| 36 | miR-30a-5p | RNA | Human | aaccuugcuuccagucgaggauguuuacaccaag |
| 37 | miR-30b | RNA | Human | uguaaacauccuacacucagcu |
| 38 | miR-30c | RNA | Human | uguaaacauccuacacucucagc |
| 39 | miR-30d | RNA | Human | uguaaacauccccgacuggaag |
| 40 | miR-30e | RNA | Human | uguaaacauccuugacuggaag |
| 41 | miR-34a | RNA | Human | uggcagugucuuagcugguugu |
| 42 | miR-29 | RNA | C. elegans | ucaccggguguaaaucagcuug |
| 43 | miR-54 | RNA | C. elegans | uacccguaaucuucauaauccgag |
| 44 | miR-92 | RNA | Human | uauugcacuugucccggccug |
| 45 | miR-93 | RNA | Human | caaagugcuguucgugcagguag |
| 46 | miR-98 | RNA | Human | ugagguaguaaguuguauuguu |
| 47 | miR-99b | RNA | Human | cacccguagaaccgaccuugcg |
| 48 | miR-100 | RNA | Human | aacccguagauccgaacuugug |
| 49 | miR-101 | RNA | Human | uacaguacugugauaacugaa |
| 50 | miR-103 | RNA | Human | agcagcauuguacagggcuauga |
| 51 | miR-106b | RNA | Human | uaaagugcugacagugcagau |
| 52 | miR-107 | RNA | Human | agcagcauuguacagggcuauca |
| 53 | miR-125a | RNA | Human | ucccugagacccuuuaaccuguga |
| 54 | miR-125b | RNA | Human | ucccugagacccuaacuuguga |
| 55 | miR-130a | RNA | Human | cagugcaauguuaaaagggcau |
| 56 | miR-130b | RNA | Human | cagugcaaugaugaaagggcau |
| 57 | miR-132 | RNA | Human | uaacagucuacagccauggucg |
| 58 | miR-133a | RNA | Human | uuugguccccuucaaccagcug |
| 59 | miR-135a | RNA | Human | uauggcuuuuuauuccuauguga |
| 60 | miR-136 | RNA | Human | acuccauuuguuuugaugaugga |
| 61 | miR-141 | RNA | Human | uaacacugucugguaaagaugg |
| 62 | miR-142-5p | RNA | Human | cauaaaguagaaagcacuac |
| 63 | miR-143 | RNA | Human | ugagaugaagcacuguagcuc |
| 64 | miR-148b | RNA | Human | ucagugcaucacagaacuuugu |
| 65 | miR-149 | RNA | Human | ucuggcuccgugucuucacuccc |
| 66 | miR-150 | RNA | Human | ucucccaacccuuguaccagug |
| 67 | miR-152 | RNA | Human | ucagugcaugacagaacuugg |
| 68 | miR-154 | RNA | Human | uagguuauccguguugccuucg |
| 69 | miR-181b | RNA | Human | aacauucauugcugucggugggu |
| 70 | miR-182 | RNA | Human | uuuggcaaugguagaacucacacu |
| 71 | miR-191 | RNA | Human | caacggaaucccaaaagcagcug |
| 72 | miR-195 | RNA | Human | uagcagcacagaaauauuggc |
| 73 | miR-196b | RNA | Human | uagguaguuuccuguuguuggg |
| 74 | miR-199a | RNA | Human | cccaguguucagacuaccuguuc |
| 75 | miR-199a* | RNA | Human | acaguagucugcacauugguua |
| 76 | miR-199b | RNA | Human | cccaguguuuagacuaucuguuc |
| 77 | miR-200a | RNA | Human | uaacacugucugguaacgaugu |
| 78 | miR-200b | RNA | Human | uaauacugccugguaaugauga |
| 79 | miR-200c | RNA | Human | uaauacugccggguaaugaugga |
| 80 | miR-203 | RNA | Human | gugaaauguuuaggaccacuag |
| 81 | miR-205 | RNA | Human | uccuucauuccaccggagucug |
| 82 | miR-210 | RNA | Human | cugugcgugugacagcggcuga |
| 83 | miR-212 | RNA | Human | uaacagucuccagucacggcc |
| 84 | miR-214 | RNA | Human | acagcaggcacagacaggcagu |
| 85 | miR-217 | RNA | Human | uacugcaucaggaacugauugga |
| 86 | miR-221 | RNA | Human | agcuacauugucugcuggguuuc |
| 87 | miR-222 | RNA | Human | agcuacaucuggcuacugggu |
| 88 | miR-224 | RNA | Human | caagucacuagugguuccguu |
| 89 | miR-238 | RNA | C. elegans | uuuguacuccgaugccauucaga |
| 90 | miR-296 | RNA | Human | agggcccccccucaauccugu |
| 91 | miR-299-5p | RNA | Human | ugguuuaccgucccacauacau |
| 92 | miR-324-5p | RNA | Human | cgcauccccuagggcauuggugu |
| 93 | miR-331 | RNA | Human | cuagguauggucccagggaucc |
| 94 | miR-339 | RNA | Human | ucccuguccuccaggagcucacg |
| 95 | miR-342 | RNA | Human | aggggugcuaucugugauuga |
| 96 | miR-361 | RNA | Human | uuaucagaaucuccagggguac |
| 97 | miR-365 | RNA | Human | uaaugccccuaaaaauccuuau |
| 98 | miR-369-3 | RNA | Human | agggagatcgaccgtgttat |
| 99 | miR-375 | RNA | Human | uuuguucguucggcucgcguga |
| 100 | miR-382 | RNA | Human | gaaguuguucgugguggauucg |
| 101 | miR-409-3p | RNA | Human | cgaauguugcucggugaaccccu |
| 102 | miR-424 | RNA | Human | cagcagcaauucauguuuugaa |
| 103 | miR-429 | RNA | Human | uaauacugucugguaaaaccgu |
| 104 | miR-449a | RNA | Human | uggcaguguauuguuagcuggu |
| 105 | miR-449b | RNA | Human | aggcaguguauuguuagcuggc |
| 106 | miR-484 | RNA | Human | ucaggcucaguccccucccgau |
| 107 | miR-495 | RNA | Human | aaacaaacauggugcacuucuu |
| 108 | miR-600 | RNA | Human | acuuacagacaagagccuugcuc |
| 109 | miR-629 | RNA | Human | uggguuuacguugggagaacu |
| 110 | miR-629* | RNA | Human | guucucccaacguaagcccagc |
| 111 | miR-660 | RNA | Human | uacccauugcauaucggaguug |
| 112 | 18 nt miRNA | RNA | Synthetic | agcguguagggauccaaa |
| 113 | 24 nt miRNA | RNA | Synthetic | ggccaacguucucaacaauaguga |
| 114 | Primer | RNA | Synthetic | rgrururcrargrargrururcrurarcrargrurcr |
| | | | | ararcrararurc |
| 115 | Primer | RNA | Synthetic | caagcagaagacggcatacgattgatggtgcctacag |
| 116 | Primer | RNA | Synthetic | aatgatacggcgaccaccgacaggttcagagttctac |
| | | | | agtccga |
| 117 | Primer | RNA | Synthetic | atcgtrargrgrcrarcrcrurgrarara |
| 118 | Primer | RNA | Synthetic | attgatggtgcctac |
| 119 | Primer | RNA | Synthetic | phosattgatggtgcctacag |
| 120 | Primer | RNA | Synthetic | phosatcgtaggcacctgaga |
| 121 | Primer | RNA | Synthetic | gcctccctcgcgccatcagatcgtaggcacctgaga |
| 122 | Primer | RNA | Synthetic | gccttgccagcccgctcagattgatggtgcctacag |
| 123 | S1359.1 Novel | RNA | Human | ggucccaucugggucgcca |
| 124 | S2574.1 Novel | RNA | Human | ugugucccauuauuggugauuu |
| 125 | S1982.1-2 Novel | RNA | Human | gcugcgucuuugugcuuuc |
| 126 | S2102.3 Novel | RNA | Human | ugucccaucugggucgcca |
| 127 | S4204.1-3 Novel | RNA | Human | gcuccagcccugccggggc |
| 128 | Novel | RNA | Human | aucccacuccugacacca |
| 129 | Novel | RNA | Human | uucucaaggaggugucguuuau |
| 130 | Novel | RNA | Human | gucccuguucgggcgcca |
| 131 | Novel | RNA | Human | agucccuucguggucgcca |
| 132 | Novel | RNA | Human | agucccaucugggucgcca |
| 133 | Novel | RNA | Human | agaggauacccuuuguauguuc |
| 134 | Novel | RNA | Human | ugucccuucguggucgcca |
| 135 | Novel | RNA | Human | uuuccggcucgcgugggugu |
| 136 | Novel | RNA | Human | gcuccagcccugccggggc |
| 137 | Novel | RNA | Human | cuuggcaccuagcaagcacuca |
| 138 | Novel | RNA | Human | ugguguggucuguuguuu |
| 139 | Novel | RNA | Human | ugcauaaggugggucca |
| 140 | Novel | RNA | Human | uguugccagucucuagg |
| 141 | Novel | RNA | Human | uuagggcccuggcuccaucucc |
| 142 | Novel | RNA | Human | cuccguuugccuguuucgcuga |
| 143 | Novel | RNA | Human | agggaggaaccaagaugg |
| 144 | Novel | RNA | Human | uggggcggagcuuccggag |
| 145 | Novel | RNA | Human | aggaaccgcagguucaga |
| 146 | Novel | RNA | Human | cuggacugagccgugcuacugg |
| 147 | Novel | RNA | Human | uagucccuuccuugaagcgguc |
| 148 | Novel | RNA | Human | agcuuccaugacuccugaugga |
| 149 | Novel | RNA | Human | uugcagcugccugggagugac |
| 150 | Novel | RNA | Human | uucuggaauucugugugaggga |
| 151 | Novel | RNA | Human | uguggugcuuauguguguguc |
| 152 | Novel | RNA | Human | cguguggugugcgccuguaa |
| 153 | Novel | RNA | Human | cgacacaaggguuugaa |
| 154 | Novel | RNA | Human | aucccaccgcugccaca |
| 155 | Novel | RNA | Human | cucgcugugaugaguga |
| 156 | Novel | RNA | Human | aacuagacugugagcuucuaga |
| 157 | Novel | RNA | Human | ccaggaauccugcuguggugga |
| 158 | Novel | RNA | Human | uguccuugcuguuuggagaua |
| 159 | Novel | RNA | Human | cggccccacgcaccaggguaaga |
| 160 | Novel | RNA | Human | gguucuuagcauaggaggucu |
| 161 | Novel | RNA | Human | uggugcaaaguaauugugguuu |
| 162 | Novel | RNA | Human | gcaguaguguagagauugguu |
| 163 | Novel | RNA | Human | uuggccccagcuccccgacc |
| 164 | Novel | RNA | Human | gagauguuaccuagcguuu |

### DETAILED DESCRIPTION OF ILLUSTRATIVE EMBODIMENTS

### I. Definitions

The terminology described herein is for the purpose of describing particular embodiments of the disclosure and is not intended to be limiting.

The singular forms “a”, “an”, and “the” as used herein include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

As used herein, “microRNA” or “miRNA” means a small, noncoding RNA sequence of 5 to 40 nucleotides in length that can be detected in a biological specimen. Some miRNAs are derived from hairpin precursors processed, for example, by the enzyme DICER to a mature species, for example, about 18-25 nucleotides, preferably 21-23 nucleotides.

In the canonical pathway, complementary codes found in the miRNA can bind to corresponding mRNA. This process can lead to inhibition of protein translation or degradation of the mRNA itself. Non-canonical modes of action have yet to be identified.

“Biological fluid” or “body fluid” can be used interchangeably and refer to a fluid isolated from a mammal. Such fluids include, but are not limited to, blood fluid, a blood fluid fraction, serum, plasma, urine, saliva, lymph, tears, pleural effusion, mucus, ascitic fluid, respiratory secretions such as bronchial secretions, amniotic fluid, cerebrospinal fluid, breast secretions, ovarian cyst fluid, and fluid isolated from a tissue.

The term “biological sample” refers to all biological fluids and excretions isolated from any given subject. In the context of the invention such samples include, but are not limited to, blood and fractions thereof, blood serum, blood plasma, urine, excreta, semen, seminal fluid, seminal plasma, prostatic fluid, pre-ejaculatory fluid (Cowper's fluid), pleural effusion, tears, saliva, sputum, sweat, biopsy, ascites, cerebrospinal fluid, amniotic fluid, lymph, marrow, cervical secretions, vaginal secretions, endometrial secretions, gastrointestinal secretions, bronchial secretions, breast secretions, ovarian cyst secretions, hair, and tissue fluid samples.

“MicroRNA Variants” are common, for example, among different animal species. In addition, variation at the 5′ and 3′ ends of miRNAs are common, and can be the result of imprecise cleavage by enzymes such as DICER during maturation. These variants demonstrate a scope of acceptable variation in the sequence of the miRNAs that does not impair function or the ability to detect the miRNA(s). Another type of variant is post-Dicer processing addition of non-templated nucleotide(s) to the 3′ end of the miRNA (these are non-templated because they do not match the human genome). The most common variants are the miRNA sequence with an extra A or U added to the 3′ end.

The terms “polynucleotide”, “oligonucleotide”, or “nucleic acid” can be used interchangeably and refer to nucleotide sequences of any length, including DNA and RNA. The nucleotides can be deoxyribonucleotides, ribonucleotides, modified nucleotides or bases, and/or their analogs, or any substrate that can be incorporated into a nucleotide sequence, for example by DNA or RNA polymerase, or by chemical reaction. Nucleic acids may be single stranded or double stranded, or may contain portions of both double and single stranded sequence. A single strand can provide a probe that hybridizes to a target sequence. A polynucleotide may comprise modified nucleotides, such as methylated nucleotides and their analogs. If present, modification to the nucleotide structure may be imparted before or after assembly of the nucleotide sequence. The sequence of nucleotides may be interrupted by non-nucleotide components. A polynucleotide may be further modified after polymerization, such as by conjugation with a labeling component. Other types of modifications include, for example, “caps”, substitution of one or more of the naturally occurring nucleotides with an analog, internucleotide modifications such as, for example, those with uncharged linkages (e.g., methyl. phosphonates, phosphotriesters, phosphoamidates, cabamates, etc.) and with charged linkages (e.g., phosphorothioates, phosphorodithioates, etc.), those containing pendant moieties, such as, for example, proteins (e.g., nucleases, antibodies, signal peptides, etc.), those with intercalators (e.g., acridine, psoralen, etc.), those containing chelators (e.g., metals, radioactive metals, boron, oxidative metals, etc.), those containing alkylators, those with modified linkages (e.g., α-anomeric nucleic acids, etc.), as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping groups moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, α-anomeric sugars, epimeric sugars such as arabinose, xyloses or lyxoses, pyranose sugars, furanose sugars, sedoheptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O) S(“thioate”), P(S)S (“dithioate”), “(O)NR₂ (“amidate”), P(O)R, P(O)OR, CO or CH₂ (“formacetal”), in which each R or R′ is independently H or substituted or unsubstituted alkyl (1-20 C). optionally containing an ether (—O—) linkage, aryl, alkenyl, cycloalkyl, cycloalkenyl or araldyl. Not all linkages in a polynucleotide need be identical.

An “isolated” polynucleotide is a nucleic acid molecule that is identified and separated from at least one contaminant nucleic acid molecule with which it is ordinarily associated in its natural source. An isolated nucleic acid molecule is other than in the form or setting in which it is found in nature. Isolated nucleic acid molecules therefore are distinguished from the specific nucleic acid molecule as it exists in natural cells.

“Complement” or “complementary” as used herein in reference to a nucleic acid sequence means Watson and Crick or Hoogsteen base pairing between nucleotides or nucleotide analogs.

“Percent (%) nucleic acid sequence identity” as used herein means the percentage of nucleotides in a candidate sequence that are identical with the nucleotides in a nucleic acid sequence of interest, after aligning the sequences and introducing gaps, if necessary, to achieve the maximum percent sequence identity. Alignment for purposes of determining percent nucleic acid sequence identity can be achieved in various ways that are within the skill in the art, for instance, using publicly available computer software such as BLAST, BLAST-2, ALIGN, ALIGN-2 or Megalign (DNASTAR) software. When comparing DNA and RNA, thymine (T) and uracil (U) may be considered equivalent.

As used herein, “differential expression” means qualitative or quantitative differences in the expression pattern of one or more polynucleotides, including miRNA, in a body fluid, cell, or tissue. Expression of the one or more polynucleotides may be upregulated, resulting in an increased amount of transcripts, or down-regulated, resulting in a decreased amount of transcripts. Expression of the one or more polynucleotides may be up-regulated or down-regulated in a particular state, such as a disease state, relative to a reference state, such as a normal state, thus permitting comparison of two or more states. The one or more polynucleotides may exhibit a pattern of expression in said body fluid, cell, or tissue that is detectable by standard techniques, including but not limited to expression arrays, quantitative reverse transcriptase PCR, northern analysis, and real-time PCR. Some of the polynucleotides may be expressed in one state but not another.

As used herein, “gene” includes any polynucleotide sequence or portion thereof with a functional role in encoding or transcribing a protein or regulating other gene expression or producing a non-coding RNA such as a miRNA. The gene may consist of all the nucleic acids responsible for encoding a functional protein or only a portion of the nucleic acids responsible for encoding or expressing a protein. The polynucleotide sequence may contain a genetic abnormality within exons, introns, initiation or termination regions, promoter sequences, other regulatory sequences or unique adjacent regions to the gene.

“Stringency” of hybridization reactions is readily determinable by one of ordinary skill in the art, and generally is an empirical calculation dependent upon probe length, washing temperature, and salt concentration. In general, longer probes can be annealed at higher temperatures, whereas shorter probes anneal well only at lower temperatures. Hybridization generally depends on the ability of denatured DNA or RNA to reanneal when complementary strands are present in an environment below their melting temperature. The higher the degree of desired homology between the probe and hybridizable sequence, the higher the relative temperature that can be used. As a result, it follows that higher relative temperatures would tend to make the reaction conditions more stringent, while lower temperatures less so.

“Stringent conditions” or “high stringency conditions” are sequence dependent and can vary dependent upon circumstances. Stringent conditions can be selected to be about 5-10° C. lower than the thermal melting point (Tₘ) for the specific sequence at a defined ionic strength pH. The Tₘ can be the temperature (under defined ionic strength, pH, and nucleic concentration) at which 50% of the probes complementary to the target hybridize to the target sequence at equilibrium. Stringent conditions include those in which the salt concentration is less than about 1.0 M sodium ion, such as about 0.01-1.0 M sodium ion concentration (or other salts) at pH 7.0 to 8.3 and the temperature is at least about 30° C. for short probes (e.g., about 10-50 nucleotides) and at least about 60° C. for long probes (e.g., greater than about 50 nucleotides). Stringent conditions can also be achieved with the addition of destabilizing agents such as formamide. Exemplary stringent condition include those that: (1) employ low ionic strength and high temperature for washing, for example 0.0 15 M sodium chloride/0.0015 M sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) overnight hybridization in a solution that employs 50% formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 pg/ml), 0.1% SDS, and 10% dextran sulfate at 42° C., with a 10 minute wash at 42° C. in 0.2×SSC (sodium chloride/sodium citrate) followed by a 10 minute high-stringency wash consisting of 0.1×SSC containing EDTA at 55° C.

“Moderately stringent conditions” may be identified as described by Sambrook et al., Molecular Cloning A Laboratory Manual, New York: Cold Spring Harbor Press, 1989, and include the use of washing solution and hybridization conditions (e.g., temperature, ionic strength and % SDS) less stringent than those described above. One example of moderately stringent conditions is overnight incubation at 37° C. in a solution comprising: 20% formamide, 5×SSC (150 mM NaCl, 15 mM trisodium citrate), 50 mM sodium phosphate (pH 7.6), 5×Denhardt's solution, 10% dextran sulfate, and 20 mg/ml denatured sheared salmon sperm DNA, followed by washing the filters in 1×SSC at about 37-50° C. The skilled artisan will recognize how to adjust the temperature, ionic strength, etc. as necessary to accommodate factors such as probe length and the like.

The term “cancer” refers to or describes the physiological condition in animals, including humans, that is typically characterized by unregulated cell growth. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases.

“Tumor”, as used herein, refers to malignant neoplastic cell growth and proliferation, and all pre-cancerous and cancerous cells and tissues.

“Carriers” as used herein include pharmaceutically acceptable carriers, exciplents, or stabilizers that are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

“Treatment” is an intervention performed with the intention of preventing the development or altering the pathology of a disease or disorder. Accordingly, “treatment” refers to both therapeutic treatment and prophylactic or preventative measures. Those in need of treatment include those already with the disease or disorder as well as those in which the disease or disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

### II. Modes for Carrying Out the Invention

The diagnostic methods described herein provide a rapid and non-invasive assay for detecting the presence of a disease or disorder in an individual, and also provide early detection and risk assessment methods. Numerous disorders can be detected according to the methods described herein, by the detection of miRNAs in a body fluid sample. In one embodiment, the disease is cancer, particularly epithelial cancers such as prostate, breast, colon, and ovarian cancers.

Many tissue based miRNAs for various diseases are known and are being rapidly discovered. Data described in the Examples below demonstrate for the first time that tissue-based diseases can be diagnosed by analysis of extracellular miRNAs, for example present in body fluids. The ability to measure tissue-based miRNAs in body fluids now provides rapid and non-invasive assays for the diagnosis of a disease or disorder, monitoring of patient response to therapeutic treatments, monitoring reoccurrence of disease, monitoring the progression of disease, and detection of risk of developing a disease or disorder. The Examples below further teach specific miRNA assays for detection of epithelial cell cancers.

In general, the diagnostic methods include determining the presence, absence, and/or amount of a miRNA in an extracellular sample, for example, body fluid obtained from an individual. The presence, absence, or amount of the miRNA in a body fluid can be compared to a control, for example a matched sample of normal body fluid, a previously analyzed sample, or a suitable standard control developed for the particular diagnostic assay.

Biological Samples

The methods of the invention comprise extracellular detection of miRNA. The miRNA is isolated from a subject's biological sample. In one embodiment, the subject's biological sample is provided, preferably a body fluid, most preferably a sample from blood. The body fluid can be blood and fractions thereof, blood serum, blood plasma, urine, excreta, semen, seminal fluid, seminal plasma, prostatic fluid, pre-ejaculatory fluid (Cowper's fluid), pleural effusion, tears, saliva, sputum, sweat, biopsy, ascites, cerebrospinal fluid, amniotic fluid, lymph, marrow, cervical secretions, vaginal secretions, endometrial secretions, gastrointestinal secretions, bronchial secretions, breast secretions, ovarian cyst secretions, hair, and tissue extract samples, including paraffin-embedded (FFPE) tissue homogenates.

RNA Isolation

In a preferred embodiment, RNA is isolated from the subject's biological sample. RNA is isolated or purified by methods known in the art, e.g., according to methods described in published laboratory manuals. The method for isolating RNA from a body fluid should be adapted to retain the small RNA fraction, for example, less than 40 nucleotides. Preferably, the miRNA isolation method retains the small RNA fraction in a background of total RNA or as an enriched fraction of RNA species. In specific embodiments, the retained RNA species are 200 nucleotides or smaller, 150 nucleotides or smaller, 100 nucleotides or smaller, 50 nucleotides or smaller, 40 nucleotides or smaller, 30 nucleotides or smaller, 25 nucleotides or smaller.

Kits for isolating RNA, and in particular miRNA, from a biological sample are known and commercially available, such as the mirVana™ miRNA isolation kits (Ambion, Austin, Tex.), e.g., the mirVana™ PARIS™ and mirVana™ miRNA isolation kits.

A biological sample can be obtained from a single individual or pooled, for example, from a group of individuals suffering from a particular disease or disorder.

Detection

MiRNA can be detected and quantified without a need to first isolate or purify RNA from the subject's biological sample. These methods include QuantiGene® (Panomics, Fremont, Calif.), and Invader® (Hologic).

Generally, miRNA can be detected and quantified from samples of isolated RNA by various methods known for mRNA, including northern blotting, ribonuclease protection assay (RPA), reverse transcription polymerase chain reaction (RT-PCR), in situ hybridization (ISH), microarray, and mass spectroscopy.

In a preferred embodiment, RNA is converted to DNA (cDNA) prior to analysis. cDNA can be generated by reverse transcription of isolated miRNA using reverse transcription conventional techniques. miRNA reverse transcription kits are known and commercially available. Examples of suitable kits include, but are not limited to, mirVana™ TaqMan® miRNA transcription kit (Ambion, Austin, Tex.) and the TaqMan® miRNA transcription kit (Applied Biosystems, Foster City, Calif.). Universal primers, or specific primers, including miRNA-specific stem-loop primers, are known and commercially available, for example, from Applied Biosystems, Ambion, and Qiagen (Valencia, Calif.).

The reverse transcript of the miRNA can be amplified using conventional PCR techniques including, but not limited to, real time PCR. Kits for quantitative real time PCR of RNA and miRNA are known and commercially available. Examples of suitable kits include, but are not limited to, the TaqMan® MiRNA Assay (Applied Biosystems) and the mirVana qRT-PCR miRNA Detection Kit (Ambion). One example of a suitable primer set is the mirVana™ qRT-PCR primer set (Ambion). The RNA can be ligated to a single stranded oligonucleotide containing universal primer sequences, a polyadenylated sequence, or adaptor sequence prior to reverse transcriptase and amplified using a primer complementary to the universal primer sequence, poly(T) primer, or primer comprising a sequence that is complementary to the adaptor sequence.

In some instances, novel miRNAs may require the development of custom qRT-PCR assays for their measurement. Custom qRT-PCR assays to measure novel miRNAs in a body fluid can be developed using a robust method that involves an extended reverse transcription primer and locked nucleic acid modified PCR. Although several qRT-PCR methods have been reported for measuring miRNAs, extended reverse transcription primer and locked nucleic acid modified PCR is likely to provide the greatest specificity. Custom miRNA assays can be tested by running it on a dilution series of chemically synthesized miRNA corresponding to the target sequence. This permits determination of the limit of detection and linear range of quantitation of each assay. Furthermore, when used as a standard curve, these data permit an estimate of the absolute abundance of endogenous miRNAs measured in body fluid samples.

Amplification curves may optionally be checked to verify that Ct values are assessed in the linear range of each amplification plot. Typically, the linear range spans several orders of magnitude. For each candidate miRNA assayed, whether known or novel, a chemically synthesized version of the miRNA can be obtained and analyzed in a dilution series to determine the limit of sensitivity of the assay, the linear range of quantitation, and to estimate the absolute abundance of the candidate miRNAs measured.

Another aspect of the invention is microarrays comprising the miRNA s of the invention. Microarrays can be used to measure the expression levels of large numbers of miRNAs simultaneously. Microarrays can be fabricated using a variety of technologies, including printing with fine-pointed pins onto glass slides, photolithography using pre-made masks, photolithography using dynamic micromirror devices, ink-jet printing, or electrochemistry on microelectrode arrays, Also useful are microfluidic TaqMan Low-Density Arrays based on an array of microfluidic qRT-PCR reactions as well as related microfluidic qRT-PCR based methods.

In one example of microarray detection, various oligonucleotides (e.g., 200+ 5′-amino-modified-C6 oligos) corresponding to human sense miRNA sequences are spotted on three-dimensional CodeLink slides (GE Health/Amersham Biosciences) at a final concentration of about 20 μM and processed according to manufacturer's recommendations. First strand cDNA synthesized from 20 μg TRIzol-purified total RNA was labeled with biotinylated ddUTP using the Enzo BioArray end labeling kit (Enzo Life Sciences Inc.) Hybridization, staining, and washing can be performed according to a modified Affymetrix Antisense genome array protocol. Axon B-4000 scanner and Gene-Pix Pro 4.0 software to scan images. Non-positive spots after background subtraction, and outliers detected by the ESD procedure are removed. The resulting signal intensity values are normalized to per-chip median values and then used to obtain geometric means and standard errors for each miRNA. Each miRNA signal was transformed to log base 2, and a one-sample t test was conducted. ^{I}ndependent hybridizations for each sample can be performed on chips with each miRNA spotted multiple times to increase the robustness of the data.

Microarrays can be used for the expression profiling of miRNAs in diseases, such as cancer. For this purpose, RNA is extracted from the body fluid and the miRNAs are size-selected from total RNA. Oligonucleotide linkers are attached to the 5′ and 3′ ends of the miRNAs and the resulting ligation products are used as templates for an RT-PCR reaction. The sense strand PCR primer can have a fluorophore attached to its 5′ end, thereby labeling the sense strand of the PCR product. The PCR product is denatured and then hybridized to the microarray. A PCR product, referred to as the target nucleic acid that is complementary to the corresponding miRNA capture probe sequence on the array will hybridize, via base pairing, to the spot at which the, capture probes are affixed. The spot will then fluoresce when excited using a microarray laser scanner. The fluorescence intensity of each spot is then evaluated in terms of the number of copies of a particular miRNA, using a number of positive and negative controls and array data normalization methods, which will result in assessment of the level of expression of a particular miRNA.

Alternatively, total RNA containing the miRNA extracted from a body fluid sample can be used directly without size-selection of the miRNAs, and the RNA is 3′ end labeled using T4 RNA ligase and a fluorophore-labeled short RNA linker. Fluorophore-labeled miRNAs complementary to the corresponding miRNA capture probe sequences on the array hybridize, via base pairing, to the spot at which the capture probes are affixed. The fluorescence intensity of each spot is then evaluated in terms of the number of copies of a particular miRNA, using a number of positive and negative controls and array data normalization methods, which will result in assessment of the level of expression of a particular miRNA.

Several types of microarrays can be employed including, but not limited to, spotted oligonucleotide microarrays, pre-fabricated oligonucleotide microarrays or spotted long oligonucleotide arrays.

Mass spectroscopy can be used to quantify miRNA using RNase mapping. Isolated RNAs can be enzymatically digested with RNA endonucleases (RNases) having high specificity (e.g., RNase T1, which cleaves at the 3′-side of all unmodified guanosine residues) prior to their analysis by MS or tandem MS (MS/MS) approaches. The first approach developed utilized the on-line chromatographic separation of endonuclease digests by reversed phase HPLC coupled directly to ESI-MS. The presence of posttranscriptional modifications can be revealed by mass shifts from those expected based upon the RNA sequence. Ions of anomalous mass/charge values can then be isolated for tandem MS sequencing to locate the sequence placement of the posttranscriptionally modified nucleoside.

Matrix-assisted laser desorption/ionization mass spectrometry (MALDI-MS) has also been used as an analytical approach for obtaining information about posttranscriptionally modified nucleosides. MALDI-based approaches can be differentiated from ESI-based approaches by the separation step. In MALDI-MS, the mass spectrometer is used to separate the miRNA.

To analyze a limited quantity of intact miRNAs, a system of capillary LC coupled with nanoESI-MS can be employed, by using a linear ion trap-orbitrap hybrid mass spectrometer (LTQ Orbitrap XL, Thermo Fisher Scientific) or a tandem-quadrupole time-of-flight mass spectrometer (QSTAR® XL, Applied Biosystems) equipped with a custom-made nanospray ion source, a Nanovolume Valve (Valco Instruments), and a splitless nano HPLC system (DiNa, KYA Technologies). Analyte/TEAA is loaded onto a nano-LC trap column, desalted, and then concentrated. Intact miRNAs were eluted from the trap column and directly injected into a C18 capillary column, and chromatographed by RP-HPLC using a gradient of solvents of increasing polarity. The chromatographic eluent is sprayed from a sprayer tip attached to the capillary column, using an ionization voltage that allows ions to be scanned in the negative polarity mode.

Additional methods for miRNA detection and measurement include, for example, strand invasion assay (Third Wave Technologies, Inc.), surface plasmon resonance (SPR), cDNA, MTDNA (metallic DNA; Advance Technologies, Saskatoon, SK), and single-molecule methods such as the one developed by US Genomics. Multiple miRNAs can be detected in a microarray format using a novel approach that combines a surface enzyme reaction with nanoparticle-amplified SPR imaging (SPRI). The surface reaction of poly(A) polymerase creates poly(A) tails on miRNAs hybridized onto locked nucleic acid (LNA) microarrays. DNA-modified nanoparticles are then adsorbed onto the poly(A) tails and detected with SPRI. This ultrasensitive nanoparticle-amplified SPRI methodology can be used for miRNA profiling at attamole levels. Advanced sequencing methods can be used as available.

Evaluation

The miRNA detected in an extracellular sample fluid can be used to identify miRNAs that are differentially expressed in an individual or pool of individuals that are suffering from a particular disease or disorder, or are at risk of developing a particular disease or disorder, as compared to a healthy individual or pool of healthy individuals. Such differentially expressed miRNAs can be used to diagnose or detect a particular disease or disorder. An ideal miRNA has low or absent expression in an extracellular sample (for example, body fluid) from a healthy individual or pool of healthy individuals versus present or high expression in disease. Conversely, a miRNA may have high expression in an extracellular sample (for example, body fluid) from a healthy individual or pool of healthy individuals, where a decrease in expression in a miRNA in a subject or pool of subjects is found associated with a disease or disorder. The miRNA may be differentially expressed within a population of patients with disease, providing information useful for prognostication, selection of therapy, or design of individualized therapies, including miRNA-directed therapies.

Candidate miRNAs can be identified by comparing extracellular miRNA expression profiles obtained from normal versus diseased individuals. In another embodiment, tissue miRNA profiles from disease individuals can be compared with extracellular (e.g., body fluids such as serum, plasma, etc.) miRNA profiles from healthy individuals, looking for miRNAs expressed in cancer but generally absent in the body fluid of healthy individuals, as described in the Examples below. Differences in the miRNA profile versus normal profile can indicate risk of disease, active disease, and response to therapy.

The isolated miRNA of the invention can be used to generate small RNA cDNA libraries. Methods for making cDNA libraries are known.

One method for identifying miRNAs comprises calculating the ratio of expression of the nonredundant miRNA sequences in the disease pool relative to the control pool (Relative Quantification (RQ)). The RQ is calculated as 2^{-ΔΔCt} where

ΔΔCt=ΔCt_{cancer}−ΔCt_{control}.

miRNA expression is represented as raw cycle threshold (Ct) values from qRT-PCR analysis. The cycle threshold values are inversely related to expression level (e.g., a lower Ct value corresponds to higher expression). ΔCt values are the Raw Ct value−Avg Ct of spiked-in synthetic miRNAs for that sample.

In an embodiment, the spiked-in synthetic miRNAs comprise C. elegans miRNAs. Generally, a RQ of about ≦0.8 or ≧1.2 indicates a differential between the presence and amount of the one or more miRNA in the disease and control samples, and identifies a candidate for the disease. For those samples analyzed by sequence data, the frequency of non-redundant miRNA sequences in the case pool or sample is compared with a control pool or sample using t-tests and/or receiver operating characteristic (ROC) curve analysis, among other statistical methods.

MiRNAs can also be diagnostically useful as a group. A p-value can be computed to determine whether or not miRNAs as a group can classify disease samples, such as cancer samples, from normal controls. Logistic regression methods can be adapted to identify optimal combinations of miRNAs. Properties of the approach include accommodating case-control sampling (unbiased in case-control sampling designs typically used to evaluate miRNAs), accommodating covariates (practical approaches must accommodate covariate adjustments due to sampling biases), and selecting miRNA candidates based on their complementarity (“variable selection” procedures allow selecting miRNAs that work better together). This framework is used when evaluating the combinations of miRNA measurements. Likelihood ratio methods can be used to assess whether certain miRNAs as a group contribute to classification above and beyond a null model.

MiRNAs can be analyzed individually and in complementarity to known miRNAs and ranked with respect to their ability to classic disease samples, such as cancer samples, from controls. This ranking can be performed in two ways. First, each miRNA can be ranked based on its performance when used alone. Secondly, the miRNAs can be ranked, one by one, on their ability to complement one or more known miRNAs. For example, a group of miRNAs isolated from serum can be ranked, one by one, on their ability to complement known ovarian cancer markers CA 125, HE4, and mesothelin, markers for which plasma samples have already been characterized. Receiver operating characteristic (ROC) curve methods can be used for the individual analysis and odds ratios can be used for analysis involving complementarity to known markers.

ROC analysis is useful to determine the extent to which each miRNA can distinguish cancer samples from controls. Differences in an individual's miRNA profile versus a normal profile can indicate risk of disease, active disease, and response to therapy that each miRNA may not indicate alone. Overall miRNAs can be ranked by their FDR (false discovery rate, or equivalently, their p-value). ROC analysis is generally performed after quantitative RT-PCR data are obtained for candidate miRNAs and appropriately normalized.

In the complementarity analysis, miRNAs are ranked based on their complementarity to known diagnostic molecules. A miRNA panel can be constructed using three or more known miRNAs to form a composite miRNA (CM) that is a weighted summary of this existing monoclonal panel; specifically, CM=f(CA125, HE4 and mesothelin). Once new CM for each miRNA is then estimated by a second logarithmic regression,

log(P)=b₁+(CM)+b₂(miRNA),

where P represents the probability of cancer. The p-value for b₂ is used to calculate FDR and also to rank miRNAs based on their significance to an existing panel.

Normalizing Measurement

The abundance of one or more miRNA can be determined by any of a number of methods, for example by calculating average Ct values. Data for each candidate miRNA in a given sample can be normalized by subtracting the Reference Ct for that sample. Normalized data can be represented by a Act value (Normalized=Average Ct of the miRNA assayed−Reference Ct). Given that Ct values are on a log₂ scale, the value 2^{ΔCt} represents linear scale expression values that can be compared directly for subsequent statistical analyses.

No established endogenous small RNA control is known for normalization of technical variations in sample processing or of potential variation in sample quality. One example of variation in sample quality of a body fluid, such as plasma, may be the presence of PCR inhibitors due to occult red blood cell lysis in plasma samples. Normalizing by matching the amount of input RNA into the reverse transcription reaction is not an appropriate approach because the RNA content of the body fluid can vary considerably and may vary with disease states. Therefore, a fixed volume of RNA eluate from a given volume of starting body sample, rather than a fixed mass of RNA, can be used as input into the reverse transcription reaction. For example, for a sample in which the starting body fluid volume was 400 pl, an input of 1.67 μl of eluted RNA (taken from a total RNA eluate volume of approximately 80.4 pl) into the reverse transcription reaction corresponds to the mass of FNA derived from approximately 8.3 μl of starting body fluid.

The data can be normalized by spiking-in one or more RNA oligonucleotide that is sufficiently distinct from the sequences normally present in the sample, for example, an miRNA obtained from a non-human species where an identical or similar human species miRNA does not exist or a synthetic sequence designed to be distinct in the sample. Any sequence that is sufficiently distinct from the sequences to be measured, but which can be measured by similar assays, is a viable candidate for a spike-in control sequence. RNAs that do not cross hybridize with probes for known human miRNA, such as C. elegans miRNAs cel-miR-39, cel-miR-54, and cel-miR-238, are spiked-in after addition of the denaturing solution to the body fluid to avoid degradation by endogenous plasma RNases. For each RNA sample, the spiked-in miRNAs can be measured using, for example, TaqMan® qRT-PCR assays.

The data can be normalized across samples using a median normalization procedure. For each sample, the Ct values obtained for the three spiked-in miRNAs are averaged to generate SpikeIn_Average_Ct value. The median of the SpikeIn_Average_Ct values obtained from all the samples to be compared is calculated (designated here as the Median_SpikeIn_Ct value). A Normalization_Factor is then calculated for each sample based on the following formula:

Normalization_Factor=1/[2̂(Median_SpikeIn_{—}Ct value)−(SpikeIn_Average_{—}Ct value of the given sample)].

The number of copies of a given miRNA in each sample, calculated using a standard curve, can be multiplied by the Normalization_Factor corresponding to the sample to obtain a normalized copy number value. In cases where it is desirable to apply normalization directly to Raw_Ct values corresponding to miRNAs of interest the Raw_Ct for a given miRNA in a given sample can be adjusted as follows: Normalized-Ct value for the miRNA in the sample=Raw_Ct value−[(SpikeIn_Average_Ct value of the given sample)−(Median_SpikeIn_Ct value)].

miRNA Variation

Mature miRNAs are described herein have specific nucleotide sequences. The sequence of each miRNA probe can also be varied, for example, due to genetic variation between individuals, i.e., SNPs, known to exist for some miRNAs and more likely to be discovered. MiRNA variation can result from changes due to RNA editing. See, for example, a single nucleotide sequence variation shown in Example 3 below, where a variant of a let-7 miRNA was found, likely due to either an SNP or RNA editing.

MiRNA variants can have up to 3 substituted or deleted nucleotides at one or both of the 5′ and 3′ ends of the miRNA. MiRNA sequences can vary at the 5′ and 3′ end (though more commonly at the 3′ end), due to various factors, including imprecise cleavage by enzymes such as the Dicer enzyme during miRNA maturation.

In an embodiment, miRNA is isolated from a body fluid and 5′- and 3′-ligated to a linker sequence, amplified by reverse transcriptase PCR, and cloned into a vector. Multiple clones can be sequenced using conventional techniques, as well as by high throughput sequencing techniques and ultra-high throughput sequencing techniques such as in vitro clonal amplification techniques and massively parallel sequencing techniques. The clones can be massively parallel sequenced using reversible terminator-based sequencing chemistry. The high throughput sequencing comprises 454 sequencing (454 Life Sciences, Branford, Conn.). In an embodiment, the high throughput sequencing comprises Solexa® sequencing (Illumina, San Diego, Calif.). In another embodiment, high-throughput sequencing of miRNAs in a population could be conducted using methods that do not require linker ligation or per amplification (and potentially in the future not even requiring cDNA synthesis), using a sequencing platform such as the Heliscope® Sequencer (Helicos, Cambridge, Mass.) in which single molecule sequencing is employed.

A complete set of reads for the miRNA sequencing data can be compiled into a set of two or more nonredundant sequences. The number of reads for each sequence reflects the relative abundance of said sequence in the sample. In an embodiment, a particular miRNA sequence must be represented by a total of at least 2 reads, 10 reads, at least 20 reads, at least 30 reads, at least 40 reads, at least 50 reads, at least 60 reads, at least 70 reads, at least 80 reads, at least 90 reads, or at least 100 to be included in the data set. miRNAs for a particular disease or disorder, such as cancer, can be identified by comparing the nonredundant sequences to a set or array of known miRNAs. miRNA that are overexpressed or underexpressed in the body fluid compared to a control are identified as candidates.

Determination of miRNA Provides Indication of Disease

Detection of miRNAs identified by the methods of the invention in a body fluid correlates with a disease or disorder. The data presented in the Examples below demonstrate a direct correlation between the presence and amount of circulating miRNA in a body fluid and disease, for example cancer. As described herein, candidate miRNAs can be identified by profiling expression of one or more miRNA in a body fluid from a healthy individual or pool of healthy individuals and comparing said profile to an miRNA expression profile derived from an individual or pool of individuals suffering from a particular disease or disorder or at risk of developing a particular disease or disorder. MiRNAs are preferentially ones that are differentially expressed in an individual or pool of individuals suffering from a particular disease or disorder or at risk of developing a particular disease or disorder compared to a healthy individual or pool of healthy individuals. In an embodiment, the miRNA comprises a Relative Quantification (RQ) of ≦0.8 or ≧1.2. The candidate miRNAs can be further correlated with a particular disease or disorder in a case-control study.

In one embodiment, candidate miRNAs include specific miRNAs shown to be absent or expressed only in very small amounts in normal human extracellular fluids such as plasma. The expression of the candidate in a diseased individual and detection of the miRNA in an extracellular fluid confirms the presence of the disease or disorder.

The miRNA of the invention can be used as diagnostic for a tumor or cancer. Examples of cancer include, but are not limited to, carcinoma, lymphoma, blastoma, sarcoma, and leukemia or lymphoid malignancies. In an embodiment, the cancer comprises squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma; anal carcinoma, penile carcinoma, melanoma, multiple myeloma and B-cell lymphoma, brain, as well as head and neck cancer, and associated metastases. In an embodiment, the cancer is an epithelial cell cancer. Examples of epithelial cell cancer include, but are not limited to, lung cancer, peritoneal cancer, hepatocellular cancer, gastric or stomach cancer including gastrointestinal cancer, pancreatic cancer, cervical cancer, ovarian cancer, liver cancer, bladder cancer, cancer of the urinary tract, breast cancer, colon cancer, rectal cancer, colorectal cancer, endometrial or uterine carcinoma, kidney or renal cancer, prostate cancer, vulval cancer, thyroid cancer, hepatic carcinoma, anal carcinoma, and penile carcinoma.

MiRNAs associated with specific cancer include those listed in Table 2 below:

TABLE 2

| SEQ ID | miRNA | Cancer |
|---|---|---|
| 48 | miR-100 | prostate |
| 54 | miR-125b | prostate |
| 63 | miR-143 | prostate |
| 90 | miR-296 | prostate |
| 111 | miR-660 | prostate |
| 235 | miR-148a | prostate |
| 110 | miR-629* | prostate |
| 61 | miR-141 | prostate, |
| | | ovarian |
| 78 | miR-200b | prostate, |
| | | ovarian |
| 79 | miR-200c | prostate, |
| | | ovarian |
| 80 | miR-203 | prostate, |
| | | ovarian |
| 104 | miR-449 | ovarian |
| 59 | miR-135a | ovarian |
| 307 | miR-135b | ovarian |
| 77 | miR-200a | ovarian |
| 103 | miR-429 | ovarian |
| 81 | miR-205 | ovarian |
| 21 | miR-20b | ovarian |
| 62 | miR-142-5p | ovarian |
| 35 | miR-29c | ovarian |
| 70 | miR-182 | ovarian |
| 123 | S1359.1 | ovarian |
| 124 | S2574.1 | ovarian |
| 125 | S1982.1-2 | ovarian |
| 126 | S2102.3 | ovarian |
| 127 | S4204.1-3 | ovarian |
| | Novel Reads | |
| | Expressed with 3 reads or more | |
| 128 | aucccacuccugacacca | ovarian |
| 129 | uucucaaggaggugucguuuau | ovarian |
| 130 | gucccuguucgggcgcca | ovarian |
| 131 | agucccuucguggucgcca | ovarian |
| 132 | agucccaucugggucgcca | ovarian |
| 133 | agaggauacccuuuguauguuc | ovarian |
| 134 | ugucccuucguggucgcca | ovarian |
| 135 | uuuccggcucgcgugggugugu | ovarian |
| 136 | gcuccagcccugccggggc | ovarian |
| 137 | cuuggcaccuagcaagcacuca | ovarian |
| 138 | ugguguggucuguuguuu | ovarian |
| 139 | ugcauaaggugggucca | ovarian |
| 140 | uguugccagucucuagg | ovarian |
| 141 | uuagggcccuggcuccaucucc | ovarian |
| 142 | cuccguuugccuguuucgcuga | ovarian |
| 143 | agggaggaaccaagaugg | ovarian |
| 144 | uggggcggagcuuccggag | ovarian |
| 145 | aggaaccgcagguucaga | ovarian |
| 146 | cuggacugagccgugcuacugg | ovarian |
| 147 | uagucccuuccuugaagcgguc | ovarian |
| 148 | agcuuccaugacuccugaugga | ovarian |
| 149 | uugcagcugccugggagugac | ovarian |
| 150 | uucuggaauucugugugaggga | ovarian |
| 151 | uguggugcuuauguguguguc | ovarian |
| 152 | cguguggugugcgccuguaa | ovarian |
| 153 | cgacacaaggguuugaa | ovarian |
| 154 | aucccaccgcugccaca | ovarian |
| 155 | cucgcugugaugaguga | ovarian |
| 156 | aacuagacugugagcuucuaga | ovarian |
| | novel reads | |
| | absent with 3 reads or more | |
| 157 | ccaggaauccugcuguggugga | ovarian |
| 158 | uguccuugcuguuuggagaua | ovarian |
| 159 | cggccccacgcaccaggguaaga | ovarian |
| 160 | gguucuuagcauaggaggucu | ovarian |
| 161 | uggugcaaaguaauugugguuu | ovarian |
| 162 | gcaguaguguagagauugguu | ovarian |
| 163 | uuggccccagcuccccgacc | ovarian |
| 164 | gagauguuaccuagcguuu | ovarian |

Computer Systems

Computer readable media comprising a plurality of protein markers, and optionally CA125, is also provided. “Computer readable media” refers to any medium that can be read and accessed directly by a computer, including but not limited to magnetic storage media, such as floppy discs, hard disc storage medium, and magnetic tape; optical storage media such as CD-ROM; electrical storage media such as RAM and ROM; and hybrids of these categories such as magnetic/optical storage media. Thus, the invention contemplates computer readable medium having recorded thereon markers identified for patients and controls. “Recorded” refers to a process for storing information on computer readable medium. The skilled artisan can readily adopt any of the presently known methods for recording information on computer readable medium to generate manufactures comprising information on a plurality of protein markers, and optionally CA125.

A variety of data processor programs and formats can be used to store information on a plurality of miRNA, including mir-100, miR-135b, miR-141, miR-148a, miR-200a, miR-200c, miR-210, miR-222, miR-375, miR-425-5p and miR-429 on computer readable medium. For example, the information can be represented in a word processing text file, formatted in commercially-available software such as WordPerfect and MicroSoft Word, or represented in the form of an ASCII file, stored in a database application, such as DB2, Sybase, Oracle, or the like. Any number of data processor structuring formats (e.g., text file or database) may be adapted in order to obtain computer readable medium having recorded thereon the marker information.

By providing the marker information in computer readable form, one can routinely access the information for a variety of purposes. For example, one skilled in the art can use the information in computer readable form to compare marker information obtained during or following therapy with the information stored within the data storage means.

The invention provides a medium for holding instructions for performing a method for determining whether a patient has a cancer or a pre-disposition to a cancer, comprising determining the presence or absence of a plurality of a miRNA, including mir-100, miR-135b, miR-141, miR-148a, miR-200a, miR-200c, miR-210, miR-222, miR-375, miR-425-5p and miR-429, and determining whether the patient has a cancer or a pre-disposition to a cancer, and optionally recommending treatment for the cancer or pre-cancer condition.

The invention also provides in an electronic system and/or in a network, a method for determining whether a subject has a cancer or a pre-disposition to a cancer associated with a plurality of miRNA, including mir-100, miR-135b, miR-141, miR-148a, miR-200a, miR-200c, miR-210, miR-222, miR-375, miR-425-5p and miR-429, determining whether the subject has a cancer or a pre-disposition to a cancer, and optionally recommending treatment for the a cancer or pre-cancer condition.

The invention further provides in a network, a method for determining whether a subject has a cancer or a pre-disposition to a cancer associated with a plurality of miRNA, including mir-100, miR-135b, miR-141, miR-148a, miR-200a, miR-200c, miR-210, miR-222, miR-375, miR-425-5p and miR-429, comprising: (a) receiving phenotypic information on the subject and information on a plurality of miRNA, including mir-100, miR-135b, miR-141, miR-148a, miR-200a, miR-200c, miR-210, miR-222, miR-375, miR-425-5p and miR-429 associated with samples from the subject; (b) acquiring information from the network corresponding to the plurality of miRNA, including mir-100, miR-135b, miR-141, miR-148a, miR-200a, miR-200c, miR-210, miR-222, miR-375, miR-425-5p and miR-429; and (c) based on the phenotypic information and information on the plurality of miRNA, including mir-100, miR-135b, miR-141, miR-148a, miR-200a, miR-200c, miR-210, miR-222, miR-375, miR-425-5p and miR-429, determining whether the subject has a cancer or a pre-disposition to a cancer; and (d) optionally recommending treatment for the cancer or pre-cancer condition.

The invention still further provides a system for identifying selected records that identify an a cancer cell. A system of the invention generally comprises a digital computer; a database server coupled to the computer; a database coupled to the database server having data stored therein, the data comprising records of data comprising a plurality of miRNA, including mir-100, miR-135b, miR-141, miR-148a, miR-200a, miR-200c, miR-210, miR-222, miR-375, miR-425-5p and miR-429, and a code mechanism for applying queries based upon a desired selection criteria to the data file in the database to produce reports of records which match the desired selection criteria.

In an aspect of the invention a method is provided for detecting a cancer cell using a computer having a processor, memory, display, and input/output devices, the method comprising the steps of:

(a) creating records of a plurality of miRNA, including mir-100, miR-135b, miR-141, miR-148a, miR-200a, miR-200c, miR-210, miR-222, miR-375, miR-425-5p and miR-429, isolated from a biological sample from a subject at risk for a cancer;

(b) providing a database comprising records of data comprising a plurality of miRNA, including mir-100, miR-135b, miR-141, miR-148a, miR-200a, miR-200c, miR-210, miR-222, miR-375, miR-425-5p and miR-429; and

(c) using a code mechanism for applying queries based upon a desired selection criteria to the data file in the database to produce reports of records of step (a) which provide a match of the desired selection criteria of the database of step (b) the presence of a match being a positive indication that the markers of step (a) have been isolated from a cell that is a cancer cell.

Manufacturing Methods

The invention contemplates a method of manufacturing a report comprising (a) contacting a biological sample, preferably a sample of blood, or material derived from the blood, with means of detecting miRNA; measuring at least one miRNA, including miR-16, miR-100, miR-141, miR-200a, miR-200c, miR-210, miR-222, miR-375, and miR-425-5p; and transforming by a computing means said measurement into a level of at least one said miRNA in the biological sample; and producing a report describing said levels of said miRNA in a tangible medium.

A major benefit of the report is to provide a means of diagnosing or measuring the presence of a disease in a clinical setting, to thereby accelerate treatment of the disease to an earlier stage than afforded by conventional diagnostic means. Ultimately a report will provide improved health care for the patient, increased certainty for the health care provider's diagnosis, decreased cost of health care for both health care managers and insurance providers, and improved public health.

Any individual having an interest in measuring the presence of a disease in a subject may cause a report to be manufactured, including a subject or patient, a medical doctor, a physician, a health management organization (HMO), a clinic, a health care provider, a health insurer, a company involved in reimbursing an insurance claim or in negotiating the cost of a diagnostic service. A pharmaceutical company may cause a report to be manufactured during a clinical trial to measure the extent of efficacy of an experimental therapy. In a health care setting, the cost of the report may be paid by an insurance provider who causes the report to conform to certain specifications which are required for payment. In the same way, the doctor or other health care provider may cause the report to contain information relevant to a diagnosis or prognosis.

The offer can be made through advertisement by computer or printed media, preferably to a group of doctors or physicians. or by specific contact with a group of medical service providers.

The offer can be made with a demand for payment. The payment may be made by the person requesting the product report. The requester can be a subject being tested, or the subject's health care provider. The payment can be received from the requester or from a third party, e.g., a group health insurance provider.

The biological sample will be provided to the manufacturer of the report. The sample may be provided directly or through agents who manage transportation within the quality needed to maintain the viability of the biological sample. The sample may be provided to the manufacturer of the report at the doctor's office or through collection centers.

The report must contain information about the levels of at least one of the miRNAs in the biological sample. The levels of the miRNA can described with respect to an absolute concentration or amount, or may be relative to normal levels in the population or relative to prior measurements in the same subject. The report may be a product manufactured from a network corresponding to (a) the plurality of miRNA, including miR-16, miR-100, miR-141, miR-200a, miR-200c, miR-210, miR-222, miR-375, and miR-425-5p; and (b) from phenotypic information, information on a plurality of miRNA, including miR-16, miR-100, miR-141, miR-200a, miR-200c, miR-210, miR-222, miR-375, and miR-425-5p, and acquired information, determining whether the subject has a cancer or a pre-disposition to a cancer; and (c) optionally recommending treatment or a treatment modality for the cancer or pre-cancer condition.

The report may instead or in addition contain a probability assessment that evaluates the relative risk of a cancer, or the extent of an existing cancer. The report may provide information to the requestor that enables a diagnosis or prognosis related to a cancer. The manufactured report can also contain a diagnosis or prognosis resulting from analysis performed by a diagnostic service provider utilizing information about the levels of at least one of the protein markers in the blood.

The report may be produced in a writing. The writing can be in a printed form, or through an electronic means. If electronic, it may be through an email account, a secure web sight or an external FTP site.

### Diagnostic Kits

Diagnostic kits adapted for the determination of miRNAs and diagnoses of disease are provided herein. Such kits may include materials and reagents adapted to specifically determine the presence and/or amount of a miRNA or group of miRNAs selected to be diagnostic of disease in a sample of body fluid. The kit can include nucleic acid molecules or probes in a form suitable for the detection of said miRNAs. The nucleic acid molecules can be in any composition suitable for the use of the nucleic acid molecules according to the instructions. The kit can include a detection component, such as a microarray, a labeling system, a cocktail of components (e.g., suspensions required for any type of PCR, especially real-time quantitative RT-PCR), membranes, color-coded beads, columns and the like. Furthermore, the kit can include a container, pack, kit or dispenser together with instructions for use.

A diagnostic kit may contain, for example, forward and reverse primers designed to amplify and detect the miRNA in body fluid. Many different PCR primers can be designed and adapted as necessary to amplify one or more miRNA that are differentially expressed in a body fluid and correlate to a particular disease or disorder. In one embodiment, the primers are designed to amplify a miRNA or group of miRNA that are differentially expressed in a body fluid of an individual having cancer or at risk of developing cancer. The diagnostic kit may also contain single stranded oligonucleotide containing universal primer sequences, polyadenylated sequences, or adaptor sequences prior and a primer complementary to said sequences. The miRNA isolated from the body fluid is ligated to the single stranded oligonucleotide containing universal primer sequence, polyadenylated sequence, or adaptor sequence prior to reverse transcription and amplified with said complementary primers. In an embodiment, the kit comprises primers that amplify one of more of miRNA, preferably at least one selected from the group consisting of miR-16, miR-100, miR-141, miR-200a, miR-200c, miR-210, miR-222, miR-375, and miR-425-5p. In another embodiment, poly-A-tailing is used to generate a sequence that can then be hybridized to a poly-T primer that is used for reverse transcription.

### Therapeutic Compositions and Methods

Unlike other genes involved in cancer, miRNA is not known to encode proteins. A miRNA can anneal to a messenger RNA containing a nucleotide sequence that complements the sequence of the miRNA. In this way, the miRNA blocks protein translation or causes degradation of the mRNA. MiRNA expression is dysregulated in human malignancies, frequently leading to overexpression or loss of expression of certain miRNAs. The function of miRNA genes depends on their targets in a specific tissue. A miRNA gene can be a tumor suppressor if in a given cell type its critical target is an oncogene, and it can be an oncogene if in a different cell type its target is a tumor-suppressor gene.

Methods of modulating a disease or disorder are provided. The disease or disorder can be a tumor or cancer. Examples of cancer include, but are not limited to, carcinoma, lymphoma, blastoma, sarcoma, and leukemia or lymphoid malignancies. In an embodiment, the cancer comprises squamous cell cancer (e.g., epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, cancer of the urinary tract, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, melanoma, multiple myeloma and B-cell lymphoma, brain, as well as head and neck cancer, and associated metastases. In an embodiment, the cancer is an epithelial cell cancer. Examples of epithelial cell cancer include, but are not limited to, lung cancer, peritoneal cancer, hepatocellular cancer, gastric or stomach cancer including gastrointestinal cancer, pancreatic cancer, cervical cancer, ovarian cancer, liver cancer, bladder cancer, cancer of the urinary tract, breast cancer, colon cancer, rectal cancer, colorectal cancer, endometrial or uterine carcinoma, kidney or renal cancer, prostate cancer, vulval cancer, thyroid cancer, hepatic carcinoma, anal carcinoma, and penile carcinoma.

The therapeutic compositions of the invention can include one or more cytotoxic agents, chemotherapeutic agents, cytokines, growth inhibitory agents, and/or immune modifiers. Such molecules are suitably present in combination in amounts that are effective for the purpose intended.

A therapeutic composition of the invention is formulated to be compatible with its intended route of administration and may thus comprise a pharmaceutically acceptable carrier, Examples of routes of administration include parenteral, e.g., intravenous, intradermal, subcutaneous, intraperitoneal, intramuscular, oral (e.g., inhalation), transdermal (topical), and transmucosal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediamine-tetraacetic acid; buffers such as acetates, citrates or phosphates, and agents for the adjustment of tonicity such as sodium chloride or dextrose. The pH can be adjusted with acids or bases, such as hydrochloric acid, or sodium hydroxide. The parenteral preparation can be enclosed in ampoules, disposable syringes, or multiple dose vials made of glass or plastic.

Another aspect of the invention is a kit containing a therapeutic composition of the invention and instructions for the prophylaxis or treatment of a disease or disorder, such as cancer. The kit may further include a device for delivering the composition to an individual, By way of example, the delivery device may be an aerosol spray device, an atomizer, a dry powder delivery device, a self-propelling solvent/powder-dispensing device, a syringe, a needle, or a dosage measuring container.

### EXAMPLES

The invention is further described by reference to the following Examples. These are intended as exemplifying embodiments of the invention, and are not to limit the invention.

### RNA Isolation Procedures

RNA was isolated from cultured cells generally using, e.g., a commercial kit, mirVana™ miRNA isolation kit (Ambion, Austin, Tex.). RNA was similarly isolated from plasma samples plasma. To allow for normalization of sample-to-sample variation in the RNA isolation step, the synthetic C. elegans miRNAs cel-miR-39, cel-miR-54 and cel-miR-238 (Qiagen, Inc., Valencia, Calif.) were added to each sample. RNA was isolated using the mirVana™ PARIS™ kit (Ambion).

### Plasma Small RNA Library Construction and Sequencing

Total plasma RNA was spiked with ³²P-labeled 18 and 24 nucleotide RNAs (Dharmacon) and gel purified by electrophoresis through denaturing polyacrylamide gel (National Diagnostics). Ligation of miRNA was performed with 3′ cloning linker 1 (IDT) and NEB T4 Rn12, followed by Illumina small-RNA cloning 5′ linker (rGrUrUrCrArGrArGrUrUrCrUrArCrArGrUrCrCrGrArCrGrArUrC) and Ambion T4 RNA ligase.

The 5′-, 3′-ligated miRNA was reverse transcribed using RT primer (CAAGCAGAAGACGGCATACGATTGATGGTGCCTACAG) and Superscript® RT III (Invitrogen). The resulting cDNA was amplified using RT primer and forward primer (AATGATACGGCGACCACCGACAGGTTCAGAGTTCTACAGTCCGA) and Platinum Taq® Polymerase High Fidelity (Invitrogen).

The PCR product was TOPO® cloned (Invitrogen) and transformed into chemically-competent E. coli. Transformants were screened by whole cell PCRusing primers specific to pCR4-TOPO®. Whole cell PCR reaction products were purified using a Qiagen 96-well PCR purification kit and sequenced by standard Sanger sequencing using a M13 forward primer.

Small RNA Library Generation from Prostate Epithelial and Stromal Cell Cultures and 454 Sequencing

Total RNA derived from two primary prostate epithelial cultures and from two primary stromal cell cultures was spiked, as described above. This RNA was 3′-ligated with T4 RNA ligase and miRNA cloning linker 1 (IDT), and 5′-ligated with T4 RNA ligase and 5′ linker (ATCGTrArGrGrCrArCrCrUrGrArArA). The fully ligated RNA was reverse transcribed using RT primer (ATTGATGGTGCCTAC). The resulting cDNA was PCR amplified with AmpliTaq® Gold using primers 5′PhosATTGATGGTGCCTACAG and 5′PhosATCGTAGGCACCTGAGA). PCR products were PCR amplified a second time using forward primer 5′GCCTCCCTCGCGCCATCAGATCGTAGGCACCTGAGA′3 and reverse primer 5′GCCTTGCCAGCCCGCTCAGATTGATGGTGCCTACAG′3. The resulting amplified DNA was sequenced by 454 Life Technologies using a Genome Sequencer FLX.

The complete set of reads for the sequencing data obtained was compiled into a set of non-redundant sequences, with the number of reads for each sequence reflecting relative abundance. The PEC dataset contained 4,721 non-redundant sequences with 60,390 reads and the PSC dataset contained 4,307 non-redundant sequences with 32,829 reads. The sequences were compared against the set of known miRNAs in miRBase Release v. 10. Reads for supersequences, sub-sequences, or sequences having significant overlap with known miRNAs were incorporated into the total number of reads given. As shown in the Examples below, 131 known miRNAs were identified in the PEC dataset and 122 known miRNAs were identified in the PSC dataset.

### Mouse Xenografts

The primary prostate epithelial and primary prostate stromal cells were cultured by methods of ordinary skill Xenografts were established in NOD/SCID mice by subcutaneous injection and blood was collected by cardiac puncture at 28 days following injection.

qRT-PCR

Plasma miRNAs were quantified using TaqMan® miRNA qRT-PCR assays with modifications. RNA was isolated from a plasma or serum sample. The miRNA was quantified by reverse transcription (RT) reaction using a TaqMan® miRNA Reverse Transcription Kit and miRNA-specific stem-loop primers (Applied BioSystems). Real-time PCR was carried out on an Applied BioSystems 7900HT thermocycler. Data were analyzed with the SDS Relative Quantification Software version 2.2.2 and with the automatic cycle threshold (Ct) setting for assigning baseline and threshold for Ct determination.

For miR-100, miR-125b, miR-141, miR-205, miR-296, miR-660, and miR-629*, the protocol was modified to include a pre-amplification step using preamplification PCR reagents (TaqMan® PreAmp Master Mix (2×) and TaqMan® miRNA Assay and a Tetrad2 Peltier Thermal Cycler (BioRad). The preamplification PCR product is diluted and introduced into the real-time PCR reaction.

Standard curves were generated for each miRNA assay using a dilution series of known input amounts of synthetic miRNA oligonucleotide corresponding to the target of the assay. The dilution series samples were run with common RT and PCR enzyme master mixes.

Synthetic single-stranded RNA oligonucleotides corresponding to the mature miRNA sequence (miRBase Release v. 10.1) were purchased from IDT (miR-629*), Sigma (miR-141 and miR-660) and Qiagen (miR-15b, miR-16, miR-19b, and miR-24). Synthetic miRNAs are input into the RT reaction over an empirically-derived range of copies to generate standard curves for each of the miRNA TaqMan® assays listed above.

In general, the lower-limit of accurate quantification for each assay is designated based on the minimal number of copies input into an RT reaction that resulted in a Ct value within the linear range of the standard curve and that was also not equivalent to or higher than a Ct obtained from an RT input of a lower copy number. A line was fit to data from each dilution series using Ct values within the linear range, from which y=mln(x)+b equations were derived for quantification of the absolute miRNA copies (x) from each sample Ct (y). The absolute copies of miRNA input into the RT reaction are converted to copies of miRNA per microliter of plasma (or serum) based on the knowledge that the material input into the RT reaction corresponds to RNA from 2.1% of the total starting volume of plasma (i.e., 1.67 μl of the total RNA eluate volume (80.4 μl on average) was input into the RT reaction).

Normalization of Experimental qRT-PCR Data

A fixed volume of RNA eluate from a given volume of starting plasma was used, rather than a fixed mass of RNA, as input into the RT reaction. Data normalization was performed using spiking-in three synthetic RNA oligonucleotides corresponding to miRNAs that do not exist in the mouse or human genomes. These RNAs were synthesized to match the sequence of three C. elegans miRNAs, cel-miR-39, cel-miR-54, and cel-miR-238 (purchased as custom RNA oligonucleotide syntheses from Qiagen)

The data were normalized across samples using a median normalization procedure. For each sample, the Ct values obtained for the three spiked-in C. elegans miRNAs were averaged to generate SpikeIn_Average_Ct value. The median of the SpikeIn_Average_Ct values obtained from all the samples to be compared was next calculated (designated here as the Median_SpikeIn_Ct value). A Normalization_Factor was then calculated for each sample based on the following formula: Normalization-Factor=1/[2̂(Median_SpikeIn_Ct value)−(SpikeIn_Average_Ct value of the given sample)].

The number of copies of a given miRNA in each sample was multiplied by the Normalization-Factor corresponding to the sample to obtain a normalized copy number value. In cases where it was desirable to apply normalization directly to Raw_Ct values corresponding to miRNAs of interest, the Raw_Ct for a given miRNA in a given sample was adjusted as follows:

Normalized_{—}Ct value for the miRNA in the sample=Raw_{—}Ct value−[(SpikeIn_Average_{—}Ct value of the given sample)−(Median_SpikeIn_{—}Ct value)]

miRNA Profiling

miRNA expression in the 22Rv1 prostate cancer cell line and in a plasma sample of RNA from a healthy human donor was profiled using TaqMan® Human mRNA Arrays (v1.0). The RNA was briefly reverse transcribed using the TaqMan® MiRNA Reverse Transcription Kit, and the TaqMan® MiRNA Multiplex RT Assays.

RNA from 22RV1 cells of eluted human plasma RNA was added to each of the eight multiplex reverse transcription reactions. The qRT-PCR was carried out on an Applied BioSystems 7900HT thermocycler. The generated data were analyzed with the SDS Relative Quantification Software version 2.2.2 having an assigned minimum threshold of 0.123518, which was above the baseline of all the assays showing a measurable amplification above background.

### Synthetic RNA Oligonucleotides

Synthetic RNA oligonucleotides used in the Examples include the synthetic RNA oligonucleotides listed in Table 3 below.

TABLE 3

| | | | SEQ |
|---|---|---|---|
| miRNA | Source | Sequence | ID NO: |
| miR-15b | Qiagen | UAGCAGCACAUCAUGGUUUACA | 14 |
| miR-16 | Qiagen | UAGCAGCACGUAAAUAUUGGCG | 15 |
| miR-19b | Qiagen | UGUGCAAAUCCAUGCAAAACUGA | 19 |
| miR-24 | Qiagen | UGGCUCAGUUCAGCAGGAACAG | 26 |
| miR-141 | Sigma | UAACACUGUCUGGUAAAGAUGG | 61 |
| miR-629* | IDT | GUUCUCCCAACGUAAGCCCAGC | 110 |
| miR-660 | Sigma | UACCCAUUGCAUAUCGGAGUUG | 111 |
| miR-29 | Qiagen | UCACCGGGUGUAAAUCAGCUUG | 42 |
| miR-54 | Qiagen | UACCCGUAAUCUUCAUAAUCCGAG | 43 |
| miR-238 | Qiagen | UUUGUACUCCGAUGCCAUUCAGA | 89 |

### Example 1

### Radiolabeling and Visualization of miRNAs/Small RNAs from Human Plasma

To determine if low molecular weight RNAs present in human plasma might include miRNAs, Small RNAs present in human plasma was isolated and characterized by size, using Radiolabeling, polyacrylamide gel electrophoresis, and phosphoimaging.

All of the detected RNA was of low molecular weight, i.e., migrating at sizes less than 100 nucleotides. Two bands were detected in the 18-24 nucleotide size range. One was observed at approximately 22 nucleotides, the most common length for known miRNAs, and the other just below the 24 nucleotide miRNA, within the size range of many known miRNAs. The detected signal was sensitive to RNase treatment but insensitive to DNase I treatment, confirming that the signal originated from RNA. These results demonstrated that human plasma contains small, detectable RNAs in the size range of miRNAs (18-24 nucleotides).

To determine if the identified small sized RNAs identified in Example 1 represented miRNAs present in human plasma, the 18-24 nucleotide RNA fraction identified in Example 1 was isolated by polyacrylamide gel electrophoresis and cloned and sequenced as described for Example 2.

### Example 2

### Generation of a Human Small RNA cDNA Library

A small RNA cDNA library was generated from RNA extracted from human plasma of a healthy donor in E. coli. Inserts from a total of 125 individual colonies yielded high-quality sequences. These sequences were compared to a reference database of known miRNA sequences (miRBase Release v.10.1) as well as to sequences available in GenBank. Of the 125 clones sequenced from this library, 73% corresponded to known miRNAs. The next most abundant species matched the sequence of the synthetic RNAs spiked-in as radiolabeled 18 and 24 nucleotide molecular size miRNAs during gel isolation steps or linker-linker dimers (27 of 125 sequences). When only endogenously-derived RNA sequences are considered, the miRNAs represent 93% (91 out of 98) of the recovered sequences. This data provides direct confirmation that mature miRNAs are present in human plasma indicating that the vast majority of the 18-24 nucleotide plasma RNA species cloned by this protocol are indeed miRNAs.

### Example 3

### Detection of Micro RNAs in Human Plasma

Quantitation of miRNA was done by qRT-PCR for miR-15b, miR-16, and miR-24 in plasma from three healthy individuals, as representative moderate- to low-abundance plasma miRNAs. These three miRNAs were all readily detected at concentrations ranging from 8,910 copies/μl of plasma to 133,970 copies/μl of plasma in the plasma of each individual, depending on the miRNA examined.

### Example 4

### Stability of miRNAs in Plasma Tested by Prolonged Incubation at Room Temperature, Repetitive Freeze-Thaw Cycles, and Endogenous RNase Activity

The stability of miRNAs in plasma was investigated using incubation at room temperature, repetitive freeze-thaw cycles, and examining stability of plasma miRNAs in the presence of endogenous RNase activity. Aliquots of plasma isolated from individuals were maintained at room temperature for 30 minutes, 1 hour, 2 hours, 4 hours, 8 hours, and 24 hours. RNA was isolation and levels of endogenous human miRNAs and of the normalization control miRNAs were measured by qRT-PCR.

The results showed that plasma samples could be incubated at room temperature for up to 24 hours, or subjected to up to eight cycles of freeze-thawing with minimal effect on the levels of miR-15b, miR-16, or miR-24.

### RNase Stability

Stability of plasma miRNA was tested in the presence of endogenous plasma RNase activity. Synthetic RNA oligonucleotides corresponding to C. elegans miRNAs cel-miR-39, cel-miR-54, and cel-miR-238 (Qiagen) were added to one plasma aliquot as a mixture of 25 fmol of each oligonucleotide. RNA was analyzed TaqMan qRTPCR. RNA was isolated from both plasma samples and the abundance of each of the three C. elegans miRNAs was measured by TaqMan® qRT-PCR as was that of three endogenous plasma miRNAs.

These results confirm the presence of RNase activity in the plasma and the sensitivity of naked miRNA to degradation. In contrast, the levels of endogenous miRNAs (i.e., miR-15b, miR-16, and miR-24) were not significantly altered in any of the experimental samples, indicating that endogenous plasma miRNAs exist in a form that is resistant to plasma RNase activity.

### Example 5

### MiRNA in Human Plasma and Serum

MiRNA measurements in serum were compared to plasma by analyzing miR-15b, miR-16, miR-19b, and miR-24 in matched samples of serum and plasma collected from a given individual at the same blood draw. The results from three different individuals demonstrate that miRNA measurements were highly correlated in both sample types. Results shown for synthetic C. elegans miRNAs spiked into each plasma or serum sample further demonstrate that experimental recovery of miRNAs and robustness of subsequent qRT-PCR is not affected by whether the sample is plasma or serum. A strong correlation between the level of miRNA in serum and plasma was found, indicating that both serum and plasma samples are suitable for investigations of miRNAs as blood-based measures of cancer.

### Example 6

### Detection of Tumor-Derived miRNAs in Plasma

Tumor-derived miRNAs enter the circulation at levels sufficient to be measurable for cancer detection as shown in a mouse prostate cancer xenograft model system using the 22Rv1 human prostate cancer cell line in NODISCID immunocompromised mice. Mice injected with 22Rv1 cells developed visible tumors. No tumors developed in the control mice.

Mice were sacrificed 28 days post-injection. Plasma and tumors were isolated and frozen. A cohort of 12 mice xenografted with 22Rv1 cells injected with Matrigel™ and 12 control mice inoculated with Matrigel™ alone were produced. Plasma was collected from xenografts and control mice 28 days later and RNA was isolated for miRNA quantitation. Plasma was assayed for the presence of MiR-15b, miR-16, and miR-24. The mature sequence of these miRNAs was conserved between mice and humans. MiRNAs miR-15b, miR-16, and miR-24 were not expressed at substantially different levels in xenograft-bearing mice, which indicated that the presence of tumor does not lead to a generalized increase in plasma miRNAs. Ct values were converted to absolute number of copies/μl plasma using a dilution series of known input quantities of synthetic target miRNA.

Identification of Tumor-Derived miRNAs

To identify candidate tumor-derived miRNAs for examination in plasma, the expression of 365 miRNAs known to be produced in 22Rv1 cells was profiled using a microfluidic TaqMan® Low-Density miRNA qRT-PCR Array (Applied BioSystems, Inc.). Two, miR-629* and miR-660, were identified as: expressed in these cells, as indicated by their low Ct values and having no known mouse homologs, therefore expected to be tumor-specific miRNAs in this setting.

Plasma levels of miR-629* and miR-660 were measured in all control and xenografied mice samples. Ct values were converted to absolute number of copies/μl plasma using a dilution series of known input quantities of synthetic target miRNA run on the same plate as the experimental samples. Next, plasma samples from control and xenograft mice were analyzed for levels of miR-629* and miR-660 using TaqMan qRT-PCR as described above. Levels of miR-629* and miR-660 were generally undetectable in the control mice, whereas they were readily detected in all the xenografted mice ranging from 10 to 1,780 copies/μl plasma for miR-629* and 5,189 to 90,783 copies/μl for miR-660. In the control (non-tumor-bearing) mice group, qRT-PCR for miR-629* or miR-660 in plasma from most animals could not detect any appreciable signal.

Levels of both miR-629* and miR-660 independently differentiated xenografted mice from controls with 100% sensitivity and 100% specificity. These results establish that tumor-derived miRNAs reach levels in plasma that can serve as a means for diagnosis of disease, such as cancer detection.

Abundance of Tumor-Derived miRNAs in Plasma of Xenografted Mice Correlates with Tumor Mass.

To understand the basis for the wide variation in miRNA abundance observed among the different xenografted mice, plasma levels of miR-629* and miR-660 were compared to tumor mass in each mouse. The abundance of tumor-derived miRNAs miR-629* and miR-660 in the plasma of mice xenografted with 22Rv1 prostate cancer cells was compared with the mass of the corresponding primary tumor. Levels of both miRNAs correlated with tumor mass (miR-629*: Spearman Rank correlation Coefficient=+0.69; miR-660: Spearman Rank Correlation Coefficient=+0.52). Accordingly, variation in miRNA abundance across animals reflects the differences in tumor burden.

### Example 7

### Biophysical Characterization of Tumor-Derived miRNAs in Mouse Plasma by Differential Centrifugation

Frozen plasma from 22Rv1 xenografted or control NOD/SCID mice from the second cohort described in Example 6 was thawed at room temperature. Two plasma pools were generated, from xenograft and control mice, by combining equal volume aliquots of plasma from each mouse within a group. Pooled plasma corresponding to xenograft or control groups were analyzed.

Pooled plasma generated from the xenograft or control groups was filtered through a 0.22 micron filter, followed by RNA extraction from the filtrate as well as from the material retained on the filter (retentate). Measurement of miR-629* and miR-660 by qRT-PCR in each of the samples demonstrated that virtually all of the tumor-derived miRNAs passed through the 0.22 micron filter. Tumor-derived miRNAs were essentially undetectable in all the samples from the control group.

Alilquots of plasma pools corresponding to xenograft and control groups were both diluted in PBS and passed through filter unit. The retentate was collected by passing 2× Denaturing Solution (Ambion) through filter unit. The PVDF filter membrane was the manually dissected from HDPE filter apparatus using a sterile scalpel and added into the retentate. Retentate was vortexed to solubilize RNA from the filter unit, centrifuged and then diluted in PBS, to which synthetic C. elegans miRNAs cel-miR-39, cel-miR-54 and cel-miR-238 were added. Filtrate samples were diluted in PBS and immediately denatured by addition of 2× Denaturing solution. Samples were vortexed after which synthetic C. elegans miRNAs cel-miR-39, cel-miR-54 and cel-miR-238 were added. RNA was isolated.

Filtrates of the xenograft animals contained 21,716 copies of miR-629* and 19,953 copies of miR-660. Retentates contained less than 50 copies and 612 copies, respectively. A retentate sample from the control group formally yielded a value of 20.9 copies; however, the Ct value was below the linear range of accurate quantification based on the standard curve and likely represents low-level background amplification.

Alternatively, plasma pools from the xenograft and control groups were subjected to a series of two centrifugations: one to pellet any intact cells remaining after the initial centrifugation used to collect plasma, followed by another at higher speed to pellet any large cell fragments. MiRNA expression was determined in the starting material, in any pelleted material obtained from each centrifugation, and in the supernatant remaining after the second centrifugation. Virtually all of the tumor-derived miRNA was present in the supernatant of the higher centrifugation. Taken together, this data indicates that tumor-derived miRNAs are not associated with intact cells or large cell fragments.

### Example 8

### Detection of Human Prostate Cancer by Detecting miRNA in Serum

To identify candidate serum miRNA s for prostate cancer, human prostate cancer miRNA expression data from the large cancer miRNA profiling dataset of Lu et al., 2005, Nature 435:834-8 showed a dataset in which 126 of the 151 miRNAs profiled had log₂ expression value >5 across prostate cancer samples. Of the 126 miRNAs, 40 miRNAs were common to a prostate cancer miRNA profiling study published by Porkka et al., 2007, Cancer Res. 67:6130-35.

A preferred miRNA cancer candidate should have low or absent expression in plasma from healthy individuals, a list of miRNAs detectable at baseline in normal human plasma was compiled by examining the list of miRNAs identified in the normal human plasma miRNA experiment reported for Example 2. MiRNA profiling of the healthy-donor plasma was performed using a TaqMan® Human mRNA Array v1.0, as described in Example 14. This is a moderate-sensitivity TaqMan® qRT-PCR-based platform for miRNA profiling.

From the list of miRNA candidates identified above, miRNAs that were sequenced in the plasma miRNA cloning study (Example 2) or that were detected at any Ct value in the normal human plasma by TaqMan® miRNA qRT-PCR Array experiment (see Table 9 in Example 14) were removed. These procedures yielded a list of six miRNA candidates: miR-100, miR-125b, miR-141, miR-143, miR-205, and miR-296, that were measured in RNA samples derived from pools of prostate cancer cases and controls as described (See Table 2).

These candidate s were analyzed in a cancer case-control cohort of serum samples collected from 25 individuals with metastatic prostate cancer and 25 healthy age-matched male control individuals. In order to efficiently screen multiple miRNA candidates, two pools of serum aliquots derived from the individuals in the cancer and control groups, respectively, were created by combining 25 samples. Each pool was mixed and RNA was isolated from each pool and screened for differential expression of the six candidate miRNAs using TaqMan qRT-PCR assays.

Results of this screen indicated that five out of six of these candidate miRNA s showed increased expression, although to varying degrees, in the prostate cancer serum pool compared to the healthy control group serum pool (Table 2). For one of the candidates (miR-205), no conclusion could be reached because miRNA levels in both pools were lower than the limit of detection of the assay (as determined by a standard curve using a dilution series of a synthetic miR-205 RNA oligonucleotide). Of all the candidates, miR-141 showed the greatest differential expression (46-fold overexpressed) in the prostate cancer pool compared to the control pool (Table 4).

TABLE 4

| | | | | | | Ratio |
|---|---|---|---|---|---|---|
| | Normal Controls | | Prostate Cancer | | | PC/Normal |
| miRNA | Raw Ct | Δ Ct | Raw Ct | Δ Ct | ΔΔ Ct | RQ |
| miR-100 | 21.8918235 | 5.899684833 | 21.0723035 | 4.431417167 | −1.439257667 | 2.77 |
| miR-125b | 22.940055 | 6.947916333 | 20.922019 | 4.281132667 | −2.666783667 | 6.35 |
| miR-141 | 25.4960765 | 9.503937833 | 20.631195 | 3.990308667 | −5.513629167 | 45.68 |
| miR-143 | 23.0193355 | 7.027196833 | 23.064886 | 6.423999667 | −0.603197167 | 1.52 |
| miR-205 | NQ | NQ | NQ | NQ | NQ | NQ |
| miR-296 | 22.9490635 | 6.38040225 | 22.3942595 | 5.82559825 | −0.554804 | 1.47 |

MiRNA expression is represented as raw cycle threshold (Ct) values from qRT-PCR analysis (using SDS software v.2.2.2). The cycle threshold values were inversely related to expression level (e.g., a lower Ct value corresponds to higher expression). Delta Ct (ΔCt) values are defined as the Raw Ct value−Avg Ct of spiked-in synthetic C. elegans miRNAs for that sample. Relative Quantification (RQ) reflects the ratio of expression in the cancer pool relative to the control pool and is calculated as 2̂(−ΔΔCt) where delta delta Ct (ΔΔCt) is defined as the Δ Ct_{Cancer}−ΔCt_{Control}.

miR-141

Focusing on miR-141 as a potential for prostate cancer, the abundance of this miRNA was next measured in each of the individual serum samples comprising the cancer and control groups. Serum levels of the prostate cancer-expressed miRNA miR-141 were measured in 25 healthy control men and in 25 patients with metastatic prostate cancer. Ct values were converted to absolute number of copies/μl serum using a dilution series of known input quantities of synthetic target miRNA run simultaneously (on the same plate) as the experimental samples.

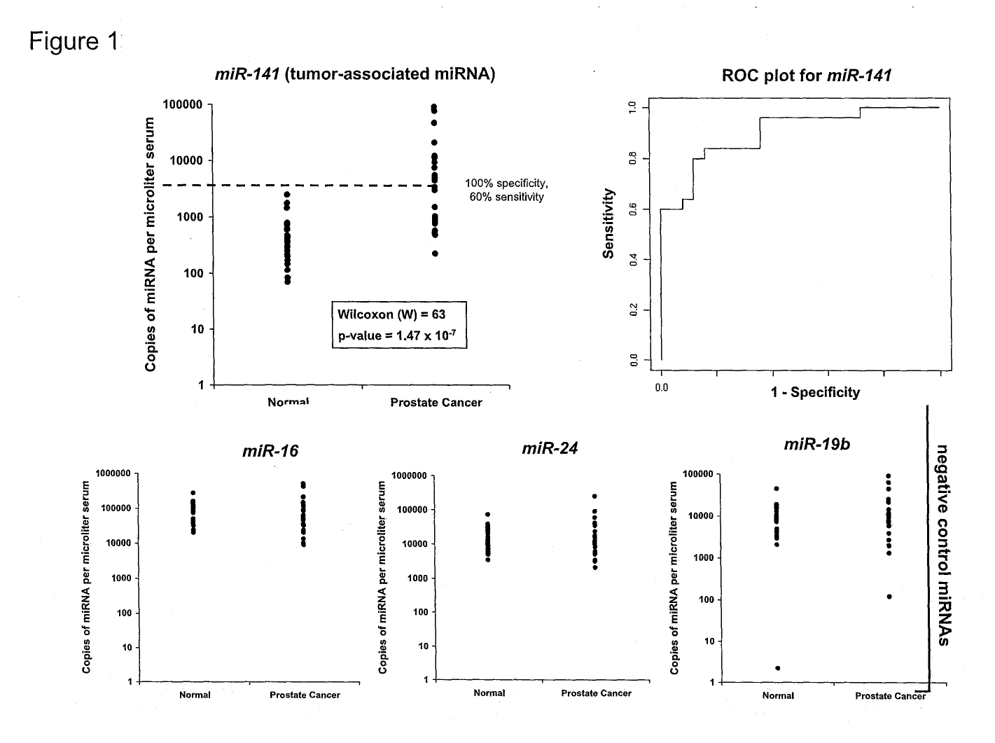

Consistent with results from the analysis of pooled samples; serum levels of miR-141 were, in general, substantially higher in cancer samples compared to controls (FIG. 1, top left panel). Comparison of the cancer and control groups by a Wilcoxon two-sample test yielded W=63 with a p-value of 1.47×10⁻⁷, confirming a significant difference in miR-141 levels between the two groups. Furthermore, serum levels of miR-141 could detect individuals with cancer with 60% sensitivity at 100% specificity (FIG. 1). Representation of the data using a Receiver Operating Characteristic (ROC) plot (FIG. 1, top right panel) reflects strong separation between the two groups.

Serum levels of non-candidate miRNAs miR-16, miR-19b, and miR-24 were not significantly different between cancer samples and controls, supporting the notion that miR-141 is specifically elevated in prostate cancer, as opposed to reflecting a nonspecific, generalized increase in serum miRNA levels in the setting of cancer. Serum levels of miR-16, miR-24 and miR-19b were measured as negative controls as these are not expected to be cancer-associated in the serum. Absolute quantification of miRNAs and data normalization were carried out as described above for miR-141 samples. Taken together, these results extend to human cancer the concept that circulating miRNAs provide useful measures of cancer.

Additional Prostate Cancer miRNA Serum Biomarkers

Additional profiling of RNA from pooled sera from 25 men with advanced prostate cancer and 25 control men without prostate cancer was performed using TLDA TaqMan® qRT-PCR of 365 microRNAs. Samples were run in duplicate. Data was normalized using measurement of spiked-in C. elegans controls spiked in after initial sample denaturation before RNA isolation. MicroRNAs with the highest fold-differences between cancer case sera and controls are listed below:

| | Normal Control Sera | Prostate Cancer Patient Sera | miRNA serum |
|---|---|---|---|
| | Pool -- Two Replicates | Pool -- Two Replicates | expression |
| | (Raw Ct values shown; | (Raw Ct values shown; | Ratio Prostate |
| miRNA | not detected = 40) | not detected = 40) | Cancer/Normal |
| miR-200c | 40, 40 | 35.26, 35.20 | 27.2 |
| miR-200a | 40, 40 | 35.23, 35.39 | 25.7 |
| miR-141 | 40, 40 | 36.88, 37.03 | 8.26 |
| miR-100 | 40, 40 | 34.17, 34.10 | 42.9 |
| miR-210 | 40, 40 | 33.85, 34.57 | 55.2 |
| miR-222 | 33.08, 34.08 | 40, 40 | 0.012 |
| miR-425-5p | 40, 40 | 35.77, 33.61 | 39.5 |
| miR-375 | 40, 40 | 31.19, 34.91 | 123 |

These combined results show changes of miR-100, miR-125b, miR-141, miR-143, miR-200a, miR-200c, miR-210, miR-222, miR-296, miR-375, and miR-425-5p during development of prostate cancer.

### Example 9

### Small RNA Library Generation from Prostate Epithelial and Stromal Cell Cultures and 454 Sequencing

To determine the relative expression of this miRNA specifically between the epithelial and stromal compartments of prostate tissue, both comparatively between the two cell types and relative to all other known miRNAs within a cell type, small RNA libraries from primary cultures of human prostate epithelial and stromal cells and subjected to massively parallel sequencing (454 sequencing).

Of the complete set of reads, the number of reads for each sequence reflected relative abundance. The prostate epithelial cell (PEC) dataset contained 4,721 nonredundant sequences with 60,390 reads and the prostate stromal cell (PSC) dataset contained 4,307 nonredundant sequences with 32,829 reads. These sequences were compared against the set of known miRNAs in miRBase Release v. 10, and is shown in Table 6. When reads in both data sets are listed in decreasing order of expression ratio, PEC/RSC, miRNA-141 and related family members miR-200b and miR200c, were readily detected in the prostate epithelial cell (PEC) dataset but strikingly absent in the prostate stromal cells.

The data showed that miR-141 and its related family members (miR-141, miR-200a, miR-200b, miR-200c, and miR-429), as well as epithelial miRNAs miR-203 and miR-205, are typically co-expressed in an epithelial cell type-specific manner in a range of common human cancers. It is expected that any of these miRNA or combinations of these can detect cancer recurrence for those cancer types where clinically validated blood markers are lacking (e.g., lung cancer, breast cancer, etc.).

Other blood-based miRNAs that are specific for particular cancer types can be discovered using the methods described herein. Each of the miRNAs listed in Table 6 is a potential miRNA for detecting epithelial cell cancer, and in particular, miR-141. 131 and 122 known miRNAs in the PEC and PSC datasets, respectively. In particular, those miRNAs expressed at some level of abundance, for example, 3 or more reads or 10 or more reads in this dataset, are potential candidate miRNAs for detection of disease in body fluids such as serum and plasma.

### Epithelial Cell MiRNAs

TABLE 6

| | PEC | Fraction PEC | PSC | Fraction PSC | Ratio: | |
|---|---|---|---|---|---|---|
| miRNA | Reads | Reads | Reads | Reads | PECf/PSCf | p-value |
| has-miR-200b | 49 | 8.11E−04 | 0 | | ∞ | <1E−6 |
| has-miR-141 | 35 | 5.80E−04 | 0 | | ∞ | <1E−6 |
| has-miR-205 | 252 | 4.17E−03 | 0 | | ∞ | <1E−6 |
| has-miR-203 | 28 | 4.64E−04 | 0 | | ∞ | 0.000009 |
| has-miR-200c | 252 | 4.17E−03 | 2 | 6.09E−05 | 68.50 | <1E−6 |
| has-let-7b | 8094 | 1.34E−01 | 1419 | 4.32E−02 | 3.10 | <1E−6 |
| has-miR-17-5p | 68 | 1.13E−03 | 13 | 3.96E−04 | 2.84 | 0.000168 |
| has-miR-30d | 8 | 1.32E−04 | 2 | 6.09E−05 | 2.17 | 0.510203 |
| has-let-7a | 11327 | 1.88E−01 | 3199 | 9.74E−02 | 1.92 | <1E−6 |
| has-miR-331 | 14 | 2.32E−04 | 4 | 1.22E−04 | 19.0 | 0.326997 |
| has-miR-19b | 154 | 2.55E−03 | 48 | 1.46E−03 | 1.74 | 0.000508 |
| has-miR-224 | 35 | 5.80E−04 | 11 | 3.35E−04 | 1.73 | 0.123317 |
| has-miR-106b | 116 | 1.92E−03 | 38 | 1.16E−03 | 1.66 | 0.006686 |
| has-miR-20a | 182 | 3.01E−03 | 62 | 1.89E−03 | 1.60 | 0.001232 |
| has-miR-19a | 29 | 4.80E−04 | 10 | 3.05E−04 | 1.58 | 0.242926 |
| has-let-7g | 78 | 1.29E−03 | 27 | 8.22E−04 | 1.57 | 0.041223 |
| has-miR-18a | 14 | 2.32E−04 | 5 | 1.52E−04 | 1.52 | 0.480909 |
| has-miR-26a | 13 | 2.15E−04 | 5 | 1.52E−04 | 1.41 | 0.626109 |
| has-miR-29b | 252 | 4.17E−03 | 100 | 3.05E−03 | 1.37 | 0.007234 |
| has-miR-149 | 7 | 1.16E−04 | 3 | 9.14E−05 | 1.27 | 1 |
| has-miR-101 | 7 | 1.16E−04 | 3 | 9.14E−05 | 1.27 | 1 |
| has-miR-23a | 106 | 1.76E−03 | 50 | 1.52E−03 | 1.15 | 0.450505 |
| has-miR-424 | 54 | 8.94E−04 | 28 | 8.53E−04 | 1.05 | 0.908165 |
| has-miR-25 | 28 | 4.64E−04 | 15 | 4.57E−04 | 1.01 | 1 |
| has-miR-130a | 88 | 1.46E−03 | 52 | 1.58E−03 | 0.92 | 0.65821 |
| has-let-7f | 3096 | 5.13E−02 | 1867 | 5.69E−02 | 0.90 | 0.000295 |
| has-miR-29a | 62 | 1.03E−03 | 38 | 1.16E−03 | 0.89 | 0.600658 |
| has-let-7e | 52 | 8.61E−04 | 32 | 9.75E−04 | 0.88 | 0569916 |
| has-miR-26b | 26 | 4.31E−04 | 16 | 4.87E−04 | 0.88 | 0.747137 |
| has-miR-100 | 29 | 4.80E−04 | 18 | 5.48E−04 | 0.88 | 0.649647 |
| has-miR-382 | 8 | 1.32E−04 | 5 | 1.52E−04 | 0.87 | 0.778951 |
| has-miR-495 | 7 | 1.16E−04 | 5 | 1.52E−04 | 0.76 | 0.763773 |
| has-miR-369-3p | 8 | 1.32E−04 | 6 | 1.83E−04 | 0.72 | 0.581543 |
| has-miR-365 | 33 | 5.46E−04 | 25 | 7.62E−04 | 0.72 | 0.217416 |
| has-miR-339 | 9 | 1.49E−04 | 7 | 2.13E−04 | 0.70 | 0.601483 |
| has-miR-16 | 159 | 2.63E−03 | 124 | 3.78E−03 | 0.70 | 0.00275 |
| has-miR-27a | 239 | 3.96E−03 | 190 | 5.79E−03 | 0.68 | 0.000113 |
| has-miR-222 | 163 | 2.70E−03 | 137 | 4.17E−03 | 0.65 | 0.00021 |
| has-miR-103 | 143 | 2.37E−03 | 121 | 3.69E−03 | 0.64 | 0.000375 |
| has-miR-34a | 72 | 1.19E−03 | 62 | 1.89E−03 | 0.63 | 0.008563 |
| has-miR-22 | 15 | 2.48E−04 | 13 | 3.96E−04 | 0.63 | 0.236842 |
| has-miR-98 | 123 | 2.04E−03 | 108 | 3.29E−03 | 0.62 | 0.000319 |
| has-miR-107 | 9 | 1.49E−04 | 8 | 2.44E−04 | 0.61 | 0.31794 |
| has-miR-30b | 46 | 7.62E−04 | 43 | 1.31E−03 | 0.58 | 0.014059 |
| has-miR-21 | 1432 | 2.37E−02 | 1394 | 4.25E−02 | 0.52 | <1E−6 |
| has-let-7i | 49 | 8.11E−04 | 49 | 1.49E−03 | 0.54 | 0.002895 |
| has-miR-30a-5p | 6 | 9.94E−05 | 6 | 1.83E−04 | 0.54 | 0.365183 |
| has-miR-136 | 13 | 2.15E−04 | 14 | 4.26E−04 | 0.50 | 0.104627 |
| has-miR-221 | 584 | 9.67E−03 | 629 | 1.92E−02 | 0.50 | <1E−6 |
| has-miR-143 | 8 | 1.32E−04 | 9 | 2.74E−04 | 0.48 | 0.134238 |
| has-miR-191 | 22 | 3.64E−04 | 25 | 7.62E−04 | 0.48 | 0.013708 |
| has-miR-125a | 6 | 9.94E−05 | 7 | 2.13E−04 | 0.47 | 0.243234 |
| has-miR-210 | 33 | 5.46E−04 | 39 | 1.19E−03 | 0.46 | 0.001179 |
| has-let-7d | 21 | 3.48E−04 | 25 | 7.62E−04 | 0.46 | 0.008358 |
| has-miR-130b | 10 | 1.66E−04 | 12 | 3.66E−04 | 0.45 | 0.072956 |
| has-miR-15b | 9 | 1.49E−04 | 11 | 3.35E−04 | 0.44 | 0.098035 |
| has-miR-154 | 9 | 1.49E−04 | 11 | 3.35E−04 | 0.44 | 0.098035 |
| has-miR-99b | 46 | 7.62E−04 | 62 | 1.89E−03 | 0.40 | 0.000003 |
| has-miR-409-3p | 10 | 1.66E−04 | 14 | 4.26E−04 | 0.39 | 0.029933 |
| has-miR-148b | 10 | 1.66E−04 | 14 | 4.26E−04 | 0.39 | 0.029933 |
| has-miR-15b | 176 | 2.91E−03 | 251 | 7.65E−03 | 0.38 | <1E−6 |
| has-miR-196b | 4 | 6.62E−05 | 6 | 1.83E−04 | 0.36 | 0.110526 |
| has-miR-324-5p | 23 | 3.81E−04 | 36 | 1.10E−03 | 0.35 | 0.000058 |
| has-let-7c | 231 | 3.83E−03 | 371 | 1.13E−02 | 0.34 | <1E−6 |
| has-miR-24 | 174 | 2.88E−03 | 174 | 9.05E−03 | 0.32 | <1E−6 |
| has-miR-342 | 4 | 6.62E−05 | 8 | 2.44E−04 | 0.27 | 0.031964 |
| has-miR-30c | 4 | 6.62E−05 | 8 | 2.44E−04 | 0.27 | 0.031964 |
| has-miR-125b | 616 | 1.02E−02 | 1261 | 3.84E−02 | 0.27 | <1E−6 |
| has-miR-361 | 4 | 6.62E−05 | 11 | 3.35E−04 | 0.20 | 0.004478 |
| has-miR-484 | 5 | 8.28E−05 | 17 | 5.18E−04 | 0.16 | 0.000069 |
| has-miR-27b | 54 | 8.94E−04 | 190 | 5.79E−03 | 0.15 | <1E−6 |
| has-miR-299-5p | 2 | 3.31E−05 | 9 | 2.74E−04 | 0.12 | 0.002142 |
| has-miR-199a* | 3 | 4.97E−05 | 14 | 4.26E−04 | 0.12 | 0.00093 |
| has-miR-152 | 5 | 8.28E−05 | 25 | 7.62E−04 | 0.11 | <1E−6 |
| has-miR-132 | 2 | 3.31E−05 | 16 | 4.87E−04 | 0.07 | 0.000004 |
| has-miR-23b | 2 | 3.31E−05 | 17 | 5.18E−04 | 0.06 | 0.000001 |
| has-miR-199a | 4 | 6.62E−05 | 260 | 7.92E−03 | 0.01 | <1E−6 |
| has-miR-199b | 0 | 0.00E+00 | 22 | 6.70E−04 | 0.00 | <1E−6 |

By providing the first evidence that tumor-derived miRNAs can enter the circulation even when originating from an epithelial cancer type (as compared to hematopoietic malignancies like lymphoma), a new diagnostic paradigm is enabled. Most importantly, this study of miR-141 in prostate cancer patients is the first demonstration that serum levels of a tumor-expressed miRNA can distinguish, with significant specificity and sensitivity, patients with cancer from healthy controls.

### Example 10

### Identification of Candidate miRNA s for Ovarian Cancer

To identify miRNAs overexpressed in serous ovarian cancers compared to primary cultures of normal human ovarian surface epithelium (HOSE), two approaches were taken: MiRNA microarrays and high-throughput sequencing (‘454 sequencing’) of small FWA cDNA libraries generated from these two sources. For the miRNA microarray experiments, microarrays bearing locked nucleic acid probes (Exiqon, Inc.) corresponding to approximately 470 known human miRNAs were used to profile RNAs derived from 16 advanced stage serous ovarian cancers selected for >70% malignant epithelium, and from primary normal HOSE cultures corresponding to four individuals. Labeling controls were added, as well as a second-channel synthetic miRNA universal reference for data normalization and quality control. Significance of differential expression was assessed using t-tests for microarray data and Fisher's exact test in the case of 454 sequencing data.

In the high-throughput sequencing approach small RNA cDNA libraries were generated from pooled serous ovarian cancer RNA and from pooled primary normal HOSE RNA using standard methods involving 5′ and 3′ linker ligation followed by reverse transcription and PCR. In a second round of PCR, sequences were incorporated into the PCR primers that enabled the products to be sequenced by a massively parallel sequencing technology known as 454 sequencing. 273,739 reads were obtained from serous ovarian cancer (intentionally sequenced more deeply) and 80,502 reads were obtained from the HOSE sample.

A bioinformatic pipeline was established for analysis of the sequences. The pipeline annotated species and also identified sequences representing novel miRNAs on the basis of a number of accepted criteria including evidence for origin from a hairpin precursor encoded in the genome. The results of such deep sequencing analysis thus identified both miRNAs that have been previously described and miRNAs of entirely novel sequence that have not been described in any previous studies. The number of times a particular sequence is recovered in the dataset relative to the total number of sequences provides an estimate of its relative abundance.

An identified miRNA with an abundance of 3 reads or more is a potential for ovarian cancer. Some s may be more useful for a particular ovarian cancer compared to another ovarian cancer. The number of times a particular sequence is recovered in the dataset (reads) relative to the total number of sequences provides a fraction, is another estimate of its relative abundance. To select a set of ten candidate plasma miRNAs for ovarian cancer detection, a list of miRNAs showing strong overexpression in serous ovarian cancer relative to normal HOSE cells was defined by combining results from miRNA microarray and high-throughput sequencing experiments. This list was further ranked on the basis of high abundance in serous ovarian cancer tissue (as reflected by the number of reads obtained in the high-throughput sequencing study). A list of the top ten candidates from the set of known miRNAs is provided in Table 7.

TABLE 7

| | #of reads in | # reads in | Fisher exact |
|---|---|---|---|
| miRNA | serous ov ca tissue | normal HOSE | test P-value |
| hsa-miR-449 | 943 | 0 | <0.001 |
| hsa-miR-141 | 817 | 0 | <0.001 |
| hsa-miR-135a | 177 | 0 | <0.001 |
| hsa-miR-200a | 177 | 0 | <0.001 |
| hsa-miR-429 | 90 | 0 | <0.001 |
| miR-205 | 84 | 0 | <0.001 |
| miR-20b | 70 | 0 | <0.001 |
| hsa-miR-142-5p | 55 | 0 | <0.001 |
| miR-29c | 55 | 0 | <0.001 |
| miR-182 | 44 | 0 | <0.001 |

In addition to the known miRNAs found to be expressed in serous ovarian cancer tissue, a number of putative miRNAs of completely novel sequence were identified by high-throughput sequencing. Five of the most abundant novel miRNAs were chosen as additional candidates for assessment in a case-control study, 29 sequences expressed in ovarian cancer with ≧3 reads, and, eight miRNAs detected in normal ovarian surface epithelial cells absent in the cancer datasets, are shown in Table 8.

TABLE 8

| | | | SEQ |
|---|---|---|---|
| | | Sequence | ID |
| Novel | S1359.1 | GGUCCCAUCUGGGUCGCCA | 123 |
| sequences | S2574.1 | UGUGUCCCAUUAUUGGUGAUUU | 124 |
| | S1982.1-2 | GCUGCGUCUUUGUGCUUUC | 125 |
| | S2102.3 | UGUCCCAUCUGGGUCGCCA | 126 |
| | S4204.1-3 | GCUCCAGCCCUGCCGGGGC | 127 |

| | | |
|---|---|---|
| Expressed with 3 | AUCCCACUCCUGACACCA | 128 |
| reads or more | UUCUCAAGGAGGUGUCGUUUAU | 129 |
| | GUCCCUGUUCGGGCGCCA | 130 |
| | AGUCCCUUCGUGGUCGCCA | 131 |
| | AGUCCCAUCUGGGUCGCCA | 132 |
| | AGAGGAUACCCUUUGUAUGUUC | 133 |
| | UGUCCCUUCGUGGUCGCCA | 134 |
| | UUUCCGGCUCGCGUGGGUGUGU | 135 |
| | GCUCCAGCCCUGCCGGGGC | 136 |
| | CUUGGCACCUAGCAAGCACUCA | 137 |
| | UGGUGUGGUCUGUUGUUU | 138 |
| | UGCAUAAGGUGGGUCCA | 139 |
| | UGUUGCCAGUCUCUAGG | 140 |
| | UUAGGGCCCUGGCUCCAUCUCC | 141 |
| | CUCCGUUUGCCUGUUUCGCUGA | 142 |
| | AGGGAGGAACCAAGAUGG | 143 |
| | UGGGGCGGAGCUUCCGGAG | 144 |
| | AGGAACCGCAGGUUCAGA | 145 |
| | CUGGACUGAGCCGUGCUACUGG | 146 |
| | UAGUCCCUUCCUUGAAGCGGUC | 147 |
| | AGCUUCCAUGACUCCUGAUGGA | 148 |
| | UUGCAGCUGCCUGGGAGUGAC | 149 |
| | UUCUGGAAUUCUGUGUGAGGGA | 150 |
| | UGUGGUGCUUAUGUGUGUGUC | 151 |
| | CGUGUGGUGUGCGCCUGUAA | 152 |
| | CGACACAAGGGUUUGAA | 153 |
| | AUCCCACCGCUGCCACA | 154 |
| | CUCGCUGUGAUGAGUGA | 155 |
| | AACUAGACUGUGAGCUUCUAGA | 156 |
| Absent in ovarian | CCAGGAAUCCUGCUGUGGUGGA | 157 |
| cancer with 3 reads | UGUCCUUGCUGUUUGGAGAUA | 158 |
| or more in normal | CGGCCCCACGCACCAGGGUAAGA | 159 |
| HOSE | GGUUCUUAGCAUAGGAGGUCU | 160 |
| | UGGUGCAAAGUAAUUGUGGUUU | 161 |
| | GCAGUAGUGUAGAGAUUGGUU | 162 |
| | UUGGCCCCAGCUCCCCGACC | 163 |
| | GAGAUGUUACCUAGCGUUU | 164 |

For example, during 454 sequencing of small RNA cDNA libraries generated from three histologic subtypes of ovarian cancer (ov/osc=serous ovarian cancer; ov/occ=clear cell ovarian cancer; ov/oec=endometrioid ovarian cancer). Except for one, the reads obtained did not match the miRBase entry exactly, but are clearly derived from the same miRNA precursor. See the alignments below, where the top line in each section indicates the RNA expected to be transcribed based on the human genome reference sequence (the miRBase-defined miRNA precursor), with the canonical mature miRNA sequence (from miRBase on or about Jul. 5, 2007) indicated in capital letters. The subsequent lines indicate sequences that were empirically identified, and the “count” indicates the number of reads corresponding to that sequence observed in a given dataset. “len” indicates length in nucleotides of the sequence, and the number preceding “miRNA” is an identifier for the sequence. As shown, mature miRNA sequences can vary at the 5′ and/or 3′ ends, even though they originate from the same longer precursor miRNA sequence. This suggests the miRNA sequence may vary at each of the 3′ or 5′ ends by up to 3 nucleotides.

TABLE 9
ov/osc_just5moreabund: has-miR-506 MIMAT0002878
gccaccaccaucagccauacuauguguagugccuuauucaggaagguguuacuuaauagauuaau
auuugUAAGGCACCCUUCUGAGUAGAguaaugugcaacauggacaacauuugugguggc

| | |
|---|---|
| GUAAGGCACCCUUCUGAGUAGA | (1378 miRNA len = 22, count 11) |
| UGUAAGGCACCCUUCUGAGUAGA | (2284 miRNA len = 23, count 6) |
| GUAAGGCACCCUUCUGAGUAG | (2723 miRNA len = 21, count 5) |

ov/occ_just5moreabund: hsa-miR-506 MIMAT0002878
gccaccaccaucagccauacuauguguagugccuuauucaggaagguguuacuuaauagauuaau
auuugUAAGGCACCCUUCUGAGUAGAguaaugugcaacauggacaacauuugugguggc

| | |
|---|---|
| GUAAGGCACCCUUCUGAGUAGA | (3766 miRNA len = 22, count 3) |
| UGUAAGGCACCCUUCUGAGUAGA | (3797 miRNA len = 23, count 3) |
| GUAAGGCACCCUUCUGAGUAG | (5908 miRNA len = 21, count 2) |
| UGUAAGGCACCCUUCUGAGUAG | (15331 miRNA len = 22, count 1) |

ov/oec_just5moreabund: hsa-miR-506 MIMAT0002878
gccaccaccaucagccauacuauguguagugccuuauucaggaagguguuacuuaauagauuaau
auuugUAAGGCACCCUUCUGAGUAGAguaaugugcaacauggacaacauuugugguggc

| | |
|---|---|
| GUAAGGCACCCUUCUGAGUAGA | (1757 miRNA len = 22, count 3) |
| GUAAGGCACCCUUCUGAGUAG | (1787 miRNA len = 21, count 3) |
| UGUAAGGCACCCUUCUGAGUAG | (10972 miRNA len = 22, count 1) |
| UGUAAGGCACCCUUCUGAGUAGA | (11275 miRNA len = 23, count 1) |

ov/osc_just5moreabund: hsa-miR-514-1 MI0003198, hsa-miR-514
MIMAT0002883
aacauguugucugugguacccuacucuggagagugacaaucauguauaauuaa
auuugAUUGACACUUCUGUGAGUAGaguaacgcaugacacguacg

| | |
|---|---|
| UGAUUGACACUUCUGUGAGUAGA | (1861 miRNA len = 23, count 7) |
| UGAUUGACACUUCUGUGAGU | (3485 miRNA len = 20, count 4) |
| UGAUUGACACUUCUGUGAGUAG | (7256 miRNA len = 22, count 2) |

ov/occ_just5moreabund: hsa-miR-514-1 MI0003198, hsa-miR-514
MIMAT0002883
aacauguugucugugguacccuacucuggagagagugacaaucauguauaauuaa
auuugAUUGACACUUCUGUGAGUAGaguaacgcaugacacguacg

| | |
|---|---|
| UGAUUGACACUUCUGUGAGUAGA | (3380 miRNA len 23, count 3) |
| AUUGACACUUCUGUGAGUAG | (5002 miRNA len 20, count = 2 CANON) |

ov/oec_just5moreabund: hsa-miR-514-1 MI0003198, hsa-miR-514
MIMAT0002883
aacauguugucugugguacccuacucuggagagugacaaucauguauaauuaa
auuugAUUGACACUUCUGUGAGUAGaguaacgcaugacacguacg

| | |
|---|---|
| UGAUUGACACUUCUGUGAGUAGA | (2047 miRNA len = 23, count 2) |
| UGAUUGACACUUCUGUGAGUAG | (3067 miRNA len = 22, count 2) |
| UUGAUUGACACUUCUGUGAGUAG | (6580 miRNA len = 23, count 1) |

### Example 11

### Known miRNAs Show Increased Abundance in Plasma from Ovarian Cancer Cases Relative to that from Healthy Controls

The expression of miR-449 and miR-141, two candidate ovarian cancer plasma s showing greatest abundance in serous ovarian cancer tissue, was examined in a plasma pool derived from 20 ovarian cancer patients and in a plasma pool derived from 20 age-matched healthy women controls. Ct values, after normalization, were lower in the ovarian cancer pool relative to the control pool by 2.30 and 3.28 cycles for miR-449 and miR-141, respectively. This corresponds to 4.9-fold over-expression of miR-449 and 9.7-fold over-expression of miR-141 in ovarian cancer patient plasma relative to plasma from the controls. These results further demonstrate that the list of identified ovarian cancer plasma s includes miRNAs useful for measuring ovarian cancer.

### Example 12

### Plasma miRNA s for Detecting Ovarian Cancer

The abundance of identified ovarian cancer microRNAs such as the known miRNAs and novel miRNAs selected as ovarian cancer plasma candidates can be measured in plasma. Custom qRT-PCR assays to measure novel micro RNAs in plasma may be developed using a robust method that involves an extended reverse transcription primer and locked nucleic acid modified PCR. Each custom miRNA assay developed can be tested with a dilution series of chemically synthesized miRNA corresponding to the target sequence to determine the limit of detection and linear range of quantitation of each assay. When used as a standard curve, this allows estimation of the absolute abundance of endogenous miRNAs in plasma samples. In addition, for each miRNA assay, two single base-mismatched synthetic control miRNAs can be generated to assess the specificity of the assays.

Moderate abundance of a miRNA can be defined as 5-99 reads in a 454 sequencing dataset. An alternative to the development of qRT-PCR assays for novel miRNAs is to utilize the proprietary TaqMan® technology of Applied Bio Systems, Inc. for developing custom assays.

Plasma is derived from patients, for example, 50 patients with advanced serous ovarian cancer (Stage IIIB or higher), collected prior to therapy. Control plasma may be collected from 50 age- and menopausal status-matched, healthy female patients from a specimen repository. All samples can be chosen to be collected within the same 3-year span. The requisite plasma samples are collected using a standardized protocol. Furthermore, the samples can also been characterized for CA-125, HE4 and mesothelin, leading protein biomarkers for ovarian cancer.

An archival aliquot of each plasma ample can first be removed and stored frozen. RNA is isolated and subjected to qRT-PCR based measurements, including three spiked-in control miRNAs. Average Ct values are calculated for each miRNA. No Template Controls (NTC) are run and evaluated for every assay. Data for each candidate miRNA in a given sample are normalized by subtracting the Reference Ct for that sample. Normalized data are therefore represented by a ΔCt value (Normalized=Average Ct of the miRNA assayed−Reference Ct). Given that Ct values are on a log₂ scale, the value 2^{ΔCt} represents linear scale expression values that can be compared directly for subsequent statistical analyses.

Amplification curves can be optionally checked to ensure that Ct values are being assessed in the linear range of each amplification plot. The linear range has spanned several orders of magnitude for most miRNA assays tested. Furthermore, for each candidate miRNA assayed, known or novel, a chemically synthesized version of the miRNA is obtained and analyzed in a dilution series to determine the limit of sensitivity of the assay, the linear range of quantitation, and to estimate the absolute abundance of the candidate miRNAs measured.

Analysis of the 15 miRNA Candidates as a Group

Taking as an example a set of 15 candidate blood-based miRNA markers for ovarian cancer, it is possible that miRNAs may be significant as classifiers as a group. The first step in the overall workflow for establishing classification can be to compute a p-value to determine whether or not as a group the 15 miRNAs can classify cancer samples from normal controls.

Specific statistical methods developed or outlined in previous publications are applied. The general theoretic framework that defines the optimal manner to combine miRNAs and, importantly, among the approaches in this framework (binary regression, i.e., logistic regression) can be adapted to identify optimal combinations. Properties of the approach include: Case-control sampling is accommodated) unbiased in case-control sampling designs typically used to evaluate miRNAs); covariates are accommodated (practical approaches must accommodate covariate adjustments due to sampling biases); and miRNA candidates can be selected based on their complementarity (“variable selection” procedures allow selecting miRNAs that work better together as a classifier of samples). This framework is used when evaluating the miRNA combinations. Because this approach is based on logistic regression, likelihood ratio methods can be used to assess whether, as a group, the 15 miRNAs contribute to classification above and beyond a null model.

The miRNAs are ranked with respect to their ability to classify cancer samples from controls. This ranking is performed in two ways. First, each miRNA is ranked based on its performance when used alone. Secondly, miRNAs, one by one, on their ability to complement known markers CA 125, HE4 and mesothelin, markers for which all plasma samples have already been characterized. Ranking uses receiver operating characteristic (ROC) curve methods for the individual analysis and odds ratios for analysis involving complementarity to known markers.

Receiver Operating Characteristic (ROC) Curve Analysis for Individual miRNAs

After quantitative RT-PCR data is obtained for candidate miRNAs and appropriately normalized, ROC analysis is useful to determine the extent to which each miRNA can distinguish cancer samples from controls. ROC analysis is chosen over t-tests and fold-change due to its specific role in measuring classification performance. Following the establishment that together the miRNAs can achieve significance, the FDR calculation may be used to rescue one or more miRNAs that does have high performance on its own. Overall miRNAs can be ranked by their FDR (or equivalently, their p-value).

Ranking miRNAs Based on Complementarity to a Panel.

MiRNAs can also be ranked based on their complementarity to existing biomarkers CA 125, HE4 and mesothelin. A miRNA panel is first estimated using these three miRNAs to form a composite miRNA (CM) that is a weighted summary of this existing monoclonal panel; specifically, CM=f(CA125, HE4 and mesothelin). One new composite miRNA for each miRNA is then estimated by a second logistic regression, log(odds)=logit(P)=b₀+₁*(CM)+b₂*(miRN˜), where P represents the probability of cancer. The p-value for b₂ is used to calculate FDR and also to rank miRNAs based on their significance to an existing panel.

### Power Calculation

The power calculations that permit classification of 50 cases and 50 controls are based on comparing the groups using a non-parametric Wilcoxon-test. The power to identify a panel based on the logistic regression is greater than the power calculated here due to its parametric nature. Power was determined by simulation. MiRNAs were simulated to have a determined AUC value of between 0.55 and 0.9 that corresponds to a miRNA having sensitivity equal to 0.1 through 0.8 at high specificity, respectively. The power (the fraction of time the Wilcoxon test statistic exceeded significance of 0.05) is shown in Table 10. For 50 cases and 50 controls, this study has 80% power to identify a miRNA (or miRNA panel) having sensitivity of 30% or greater. Power is higher for t-tests. The power of the logistic regression to classify cases from controls is approximate to this. Thus, this study has the power to detect an overall classification ability should the miRNAs together achieve 30% sensitivity, even if no individual miRNA exceeds that performance.

Table 10 shows a summary of power to detect miRNAs of a given sensitivity based on numbers of cases and controls using the Mann-Whitney test.

TABLE 10

| | | Number of Cases/Controls | | | | | | | | | |
|---|---|---|---|---|---|---|---|---|---|---|---|
| | | 100/100 | | 75/75 | | 50/50 | | 25/25 | | 10/10 | |
| AUC | Sensitivity | 0.05 | 0.10 | 0.05 | 0.10 | 0.05 | 0.10 | 0.05 | 0.10 | 0.05 | 0.10 |
| 0.55 | 0.1 | 0.22 | 0.34 | 0.14 | .24 | 0.12 | 0.19 | 0.06 | 0.12 | 0.05 | 0.09 |
| 0.60 | 0.2 | 0.70 | 0.81 | 0.56 | .71 | 0.39 | 0.54 | 0.20 | 0.31 | 0.06 | 0.13 |
| 0.65 | 0.3 | 0.99 | 1.00 | 0.94 | .97 | 0.80 | 0.89 | 0.38 | 0.54 | 0.14 | 0.23 |
| 0.70 | 0.4 | 1.00 | 1.00 | 1.00 | 1.00 | 0.99 | 1.00 | 0.75 | 0.87 | 0.24 | 0.39 |
| 0.75 | 0.5 | 1.00 | 1.00 | 1.00 | 1.00 | 1.00 | 1.00 | 0.97 | 0.99 | 0.43 | 0.62 |
| 0.80 | 0.6 | 1.00 | 1.00 | 1.00 | 1.00 | 1.00 | 1.00 | 1.00 | 1.00 | 0.67 | 0.88 |
| 0.85 | 0.7 | 1.00 | 1.00 | 1.00 | 1.00 | 1.00 | 1.00 | 1.00 | 1.00 | 0.94 | 0.99 |
| 0.90 | 0.8 | 1.00 | 1.00 | 1.00 | 1.00 | 1.00 | 1.00 | 1.00 | 1.00 | 1.00 | 1.00 |

### Example 13

### Identification of Plasma miRNA s by Sequencing of Small RNA cDNA Libraries Generated from Plasma

### Technology for Ultra-High-Throughput Sequencing.

For the discovery of novel miRNA candidate s directly from plasma, new, ultra-high-throughput sequencing technology (uHTS) (i.e., Solexa®; known formally as the Illumina® Genome Analyzer) is used to sequence plasma small RNA cDNA libraries generated from plasma obtained from women with ovarian cancer and from healthy women controls. Additional information on the platform is available at www.Illumina.com and more specifically at http://www.illumina.com/pages.ilmn?ID=204. In contrast to high-throughput 454 sequencing technology (described above) that generates typically about 200,000 sequences from one sample in one run, the Solexa® platform generates 5 million reads per sample from each of 8 samples in a single run.

Plasma RNA Isolation and Synthetic miRNA Normalization Controls to be Spiked in.

About 2.5 mL of plasma from each individual can be used. This amount is calculated based on the amount of small RNA used in our experiments with serous ovarian cancer tissue described in Example 10. In the tissue experiments, we began with 100 pg of total RNA, of which 0.1% (or 100 ng) is estimated to be the small RNA fraction. In plasma, we have found that all the RNA is present as small RNA and the yield after RNA isolation is typically 200 ng from 1 mL of input plasma. A starting amount of 2.5 mL therefore represents a total of 500 ng of small RNA input that is five times as much small RNA input as we have successfully used in past library generation experiments.

Plasma RNA is isolated using the method described in the above Examples, quality controls for FWA isolation and subsequent library generation a pool of 40 non-human miRNAs (derived from the C. elegans and Arabidopsis genomes) that are highly divergent from any known human miRNAs and do not have any matches to the human genome sequence can be as an alternate control. Based on our earlier 454 studies in which synthetic small RNAs were spiked in as size makers for PAGE, we estimate that 20 nmol per sample should yield final datasets in which 0.5-1% of the sequences correspond to the spiked-in non-human miRNAs.

Because a fixed amount of these will be spiked into each sample, the sequence abundance data corresponding to these controls will help provide a measure of technical variability in library generation and sequencing and will help to normalize the data between samples.

### Example 14

### Detection of Underexpressed Plasma miRNA s Correlate with Detection of Ovarian Cancer

Novel sequences were identified from the 454 sequencing and had three reads or more. These miRNAs were detected in normal ovarian surface epithelial cells but absent in the cancer datasets. These novel miRNAs can be useful for detecting ovarian cancer. (Table 11.)

TABLE 11

| SEQ ID NO | Sequence |
|---|---|
| 157 | CCAGGAAUCCUGCUGUGGUGGA |
| 158 | UGUCCUUGCUGUUUGGAGAUA |
| 159 | CGGCCCCACGCACCAGGGUAAGA |
| 160 | GGUUCUUAGCAUAGGAGGUCU |
| 161 | UGGUGCAAAGUAAUUGUGGUUU |
| 162 | GCAGUAGUGUAGAGAUUGGUU |
| 163 | UUGGCCCCAGCUCCCCGACC |
| 164 | GAGAUGUUACCUAGCGUUU |

Particular ovarian cancer miRNAs (such as the 8 miRNAs in Table 11) can be measured in plasma (for example, by qRT-PCR), and their under-expression can be correlated with ovarian cancer. Under-expression can be defined as an absence of miRNA expression or less expression in ovarian cancer cells compared to basal levels of miRNA expressed in normal ovarian surface epithelial cells.

Commercially available TaqMan® qRT-PCR assays for the miRNAs can be used, and custom qRT-PCR assays can be developed for the novel miRNA s for their measurement, as discussed previously in Example 12. Biological samples can be obtained and analyzed as discussed in Example 12. Analysis can be also be performed as previously described. However, under-expressed miRNAs would be absent or quantifiably less abundant in a biological sample, particularly a plasma sample, from a subject with ovarian cancer compared to a biological sample from a subject not having ovarian cancer.

### Example 14

### Identification of Plasma miRNA s of Disease

An ideal extracellular of a disease or disorder, for example, to be detected in extracellular fluid such as plasma or serum, is one that is highly disease specific. The extracellular ideal measure would based on very low or absent expression in normal extracellular fluid, and its detection and/or abundance in extracellular fluid that would be indicative of disease.

The results of TLDA TaqMan® qRT-PCR assay of human normal blood (as described in Examples 2 and 8) demonstrated that many of the assayed miRNAs were not detectable in normal plasma sample. Where these known miRNAs are expressed in a disease or disorder, e.g., expressed by a cancer such as prostate or ovarian cancer, and not normally found in body fluids such as plasma of healthy individuals, the miRNA is an ideal extracellular for the disorder. Those miRNAs not detected in plasma of healthy individuals are candidates for ideal measures of disease. These miRNAs can be evaluated for diagnosing the disease from analysis of miRNA in extracellular fluids of individuals suffering from the disease or disorder.

As shown in Table 12, many of the assayed microRNAs were absent (not detectable) in the normal plasma sample. Where these known microRNAs are expressed in a disease or disorder, e.g., expressed by a cancer such as prostate or ovarian cancer, and not normally found in body fluids such as plasma of healthy individuals, the microRNA is an ideal extracellular biomarker for the disorder.

Reviewing the data of Table 12 below, those microRNAs listed as not detected in plasma of healthy individuals or detected at low abundance (Ct value equal to or greater than 30) in plasma of healthy individuals are candidates for ideal biomarkers of disease. These microRNAs can be evaluated for correlation to disease and detection in extracellular fluids of individuals suffering from the disease or disorder.

TABLE 12

| SEQ ID | miRNA | Sequence | Ct |
|---|---|---|---|
| 165 | hsa-miR-223-4373075 | AGCUACAUCUGGCUACUGGGUCUC | 21.387175 |
| 166 | hsa-miR-16-4373121 | UAGCAGCACGUAAAUAUUGGCG | 23.676111 |
| 167 | hsa-miR-126-4378064 | CAUUAUUACUUUUGGUACGCG | 25.18676 |
| 168 | hsa-miR-26a-4373070 | UUCAAGUAAUCCAGGAUAGGC | 25.349148 |
| 169 | hsa-miR-24-4373072 | UGGCUCAGUUCAGCAGGAACAG | 25.352934 |
| 170 | hsa-miR-19b-4373098 | UGUGCAAAUCCAUGCAAAACUGA | 25.541594 |
| 171 | hsa-miR-142-3p-4373136 | UGUAGUGUUUCCUACUUUAUGGA | 25.777431 |
| 172 | hsa-miR-92-4373013 | UCUUUGGUUAUCUAGCUGUAUGA | 26.029703 |
| 173 | hsa-miR-26b-4373069 | UUCAAGUAAUUCAGGAUAGGUU | 26.197817 |
| 174 | hsa-miR-191-4373109 | CAACGGAAUCCCAAAAGCAGCU | 26.402868 |
| 175 | hsa-miR-20a-4373286 | UAAAGUGCUUAUAGUGCAGGUAG | 26.709143 |
| 176 | hsa-miR-146a-4373132 | UGAGAACUGAAUUCCAUGGGUU | 26.751621 |
| 177 | hsa-miR-484-4381032 | UCAGGCUCAGUCCCCUCCCGAU | 26.773163 |
| 178 | hsa-miR-222-4373076 | AGCUACAUUGUCUGCUGGGUUUC | 27.214977 |
| 179 | hsa-miR-93-4373012 | UAAAGCUAGAUAACCGAAAGU | 27.30701 |
| 180 | hsa-miR-486-4378096 | UCCUGUACUGAGCUGCCCCGAG | 27.477003 |
| 181 | hsa-miR-186-4373112 | CAAAGAAUUCUCCUUUUGGGCUU | 27.807243 |
| 182 | hsa-miR-126-4373269 | UCGUACCGUGAGUAAUAAUGC | 27.860714 |
| 183 | hsa-miR-30b-4373290 | UGUAAACAUCCUACACUCAGCU | 28.025436 |
| 184 | hsa-miR-15b-4373122 | UAGCAGCACAUCAUGGUUUACA | 28.029036 |
| 185 | hsa-miR-30c-4373060 | UGUAAACAUCCUACACUCUCAGC | 28.051128 |
| 186 | hsa-miR-19a-4373099 | UGUGCAAAUCUAUGCAAAACUGA | 28.140797 |
| 187 | hsa-miR-221-4373077 | CCACACCGUAUCUGACACUUU | 28.160389 |
| 188 | hsa-miR-151-4373179 | ACUAGACUGAAGCUCCUUGAGG | 28.363777 |
| 189 | hsa-miR-142-5p-4373135 | CAUAAAGUAGAAAGCACUAC | 28.433594 |
| 190 | hsa-miR-103-4373158 | AGCAGCAUUGUACAGGGCUAUGA | 28.643219 |
| 191 | hsa-miR-17-5p-4373119 | CAAAGUGCUUACAGUGCAGGUAGU | 28.771618 |
| 192 | hsa-miR-30a-5p-4373061 | UGUAAACAUCCUCGACUGGAAG | 28.948341 |
| 193 | hsa-miR-140-4373138 | AGUGGUUUUACCCUAUGGUAG | 29.038239 |
| 194 | hsa-miR-342-4373040 | UCUCACACAGAAAUCGCACCCGUC | 29.161871 |
| 195 | hsa-miR-425-5p-4380926 | AAUGACACGAUCACUCCCGUUGA | 29.179981 |
| 196 | hsa-miR-27a-4373287 | UUCACAGUGGCUAAGUUCCGC | 29.258652 |
| 197 | hsa-miR-320-4373055 | UAUUGCACAUUACUAAGUUGC | 29.308796 |
| 198 | hsa-miR-106b-4373155 | UAAAGUGCUGACAGUGCAGAU | 29.49641 |
| 199 | hsa-miR-374-4373028 | UUAUAAUACAACCUGAUAAGUG | 29.52162 |
| 200 | hsa-miR-30d-4373059 | UGUAAACAUCCCCGACUGGAAG | 29.610056 |
| 201 | hsa-miR-146b-4373178 | UGAGAACUGAAUUCCAUAGGCU | 29.699888 |
| 202 | hsa-miR-432-4373280 | UCUUGGAGUAGGUCAUUGGGUGG | 29.779055 |
| 203 | hsa-miR-197-4373102 | UUCACCACCUUCUCCACCCAGC | 29.831097 |
| 204 | hsa-miR-331-4373046 | GCAAAGCACACGGCCUGCAGAGA | 29.86672 |
| 205 | hsa-miR-127-4373147 | UCGGAUCCGUCUGAGCUUGGCU | 29.948317 |
| 206 | hsa-let-7g-4373163 | UGAGGUAGUAGUUUGUACAGU | 29.965107 |
| 207 | hsa-miR-125a-4373149 | UCCCUGAGACCCUUUAACCUGUG | 29.986898 |
| 208 | hsa-miR-328-4373049 | CUGGCCCUCUCUGCCCUUCCGU | 30.116018 |
| 209 | hsa-miR-301-4373064 | CAGUGCAAUAGUAUUGUCAAAGC | 30.40973 |
| 210 | hsa-miR-382-4373019 | GAAGUUGUUCGUGGUGGAUUCG | 30.51329 |
| 211 | hsa-miR-21-4373090 | ACCAUCGACCGUUGAUUGUACC | 30.570774 |
| 212 | hsa-miR-28-4373067 | AAGGAGCUCACAGUCUAUUGAG | 30.670748 |
| 213 | hsa-miR-425-4373202 | AUCGGGAAUGUCGUGUCCGCC | 30.68779 |
| 214 | hsa-miR-20b-4373263 | CAAAGUGCUCAUAGUGCAGGUAG | 30.689703 |
| 215 | hsa-let-7b-4373168 | UGAGGUAGUAGGUUGUGUGGUU | 30.78085 |
| 216 | hsa-miR-195-4373105 | UAGCAGCACAGAAAUAUUGGC | 30.927505 |
| 217 | hsa-miR-134-4373141 | UGUGACUGGUUGACCAGAGGG | 30.999826 |
| 218 | hsa-miR-130a-4373145 | CAGUGCAAUGUUAAAAGGGCAU | 31.000528 |
| 219 | hsa-miR-130b-4373144 | CAGUGCAAUGAUGAAAGGGCAU | 31.078936 |
| 220 | RNU48-4373383 | GAUGACCCCAGGUAACUCUGAGUGUG | 31.113333 |
| | | UCGCUGAUGCCAUCACCGCAGCGCUC | |
| | | UGACC | |
| 221 | hsa-miR-25-4373071 | CAUUGCACUUGUCUCGGUCUGA | 31.12904 |
| 222 | hsa-miR-485-3P-4378095 | GUCAUACACGGCUCUCCUCUCU | 31.133604 |
| 220 | RNU48-4373383 | | 31.251173 |
| 220 | RNU48-4373383 | | 31.336447 |
| 223 | hsa-miR-411-4381013 | UAGUAGACCGUAUAGCGUACG | 31.392809 |
| 220 | RNU48-4373383 | | 31.430758 |
| 224 | hsa-miR-335-4373045 | UCAAGAGCAAUAACGAAAAAUGU | 34.442823 |
| 220 | RNU48-4373383 | | 31.452744 |
| 220 | RNU48-4373383 | | 31.479742 |
| 220 | RNU48-4373383 | | 31.515448 |
| 225 | hsa-let-7a-4373169 | UGAGGUAGUAGGUUGUAUAGUU | 31.531221 |
| 226 | hsa-miR-181d-4373180 | AACAUUCAUUGUUGUCGGUGGGUU | 31.545853 |
| 227 | hsa-miR-32-4373056 | GCACAUUACACGGUCGACCUCU | 31.569336 |
| 228 | hsa-miR-376A-4373026 | AUCAUAGAGGAAAAUCCACGU | 31.573658 |
| 229 | hsa-miR-152-4373126 | UCAGUGCAUGACAGAACUUGGG | 31.622772 |
| 230 | hsa-miR-532-4380928 | CAUGCCUUGAGUGUAGGACCGU | 31.661785 |
| 231 | hsa-miR-451-4373209 | AAACCGUUACCAUUACUGAGUUU | 31.692917 |
| 232 | hsa-miR-324-3P-4373053 | CCACUGCCCCAGGUGCUGCUGG | 31.720333 |
| 233 | hsa-miR-410-4378093 | AAUAUAACACAGAUGGCCUGU | 31.905563 |
| 234 | hsa-miR-340-4373041 | UCCGUCUCAGUUACUUUAUAGCC | 31.926765 |
| 235 | hsa-miR-148a-4373130 | UCAGUGCACUACAGAACUUUGU | 31.928728 |
| 236 | hsa-miR-199a-4378068 | UACAGUAGUCUGCACAUUGGUU | 32.002617 |
| 237 | hsa-miR-487b-4378102 | AAUCGUACAGGGUCAUCCACUU | 32.159157 |
| 220 | RNU48-4373383 | | 32.170868 |
| 238 | hsa-miR-224-4373187 | CAAGUCACUAGUGGUUCCGUUUA | 32.18172 |
| 239 | hsa-miR-18a-4373118 | UAAGGUGCAUCUAGUGCAGAUA | 32.210087 |
| 240 | hsa-miR-29a-4373065 | UAGCACCAUCUGAAAUCGGUU | 32.36902 |
| 241 | hsa-miR-30e-3p-4373057 | CUUUCAGUCGGAUGUUUACAGC | 32.405838 |
| 242 | hsa-miR-200c-4373096 | UAAUACUGCCGGGUAAUGAUGG | 32.412086 |
| 243 | hsa-miR-22-4373079 | UGUCAGUUUGUCAAAUACCCC | 32.444557 |
| 244 | hsa-miR-339-4373042 | UCCCUGUCCUCCAGGAGCUCA | 32.55535 |
| 245 | hsa-miR-155-4373124 | UUAAUGCUAAUCGUGAUAGGGG | 32.556374 |
| 246 | hsa-miR-433-4373205 | AUCAUGAUGGGCUCCUCGGUGU | 32.639957 |
| 247 | hsa-miR-192-4373108 | CUGACCUAUGAAUUGACAGCC | 32.696526 |
| 248 | hsa-miR-27b-4373068 | UUCACAGUGGCUAAGUUCUGC | 32.729248 |
| 249 | hsa-miR-99b-4373007 | CACCCGUAGAACCGACCUUGCG | 32.960518 |
| 250 | hsa-miR-23b-4373073 | AUCACAUUGCCAGGGAUUACC | 32.97151 |
| 251 | hsa-miR-491-4373216 | AGUGGGGAACCCUUCCAUGAGGA | 33.038338 |
| 252 | RNU44-4373384 | CCUGGAUGAUGAUAGCAAAUGCUGAC | 33.05032 |
| | | UGAACAUGAAGGUCUUAAUUAGCUCUA | |
| | | ACUGACU | |
| 253 | hsa-miR-423-4373015 | AGCUCGGUCUGAGGCCCCUCAG | 33.054043 |
| 254 | hsa-miR-133b-4373172 | UUGGUCCCCUUCAACCAGCUA | 33.080162 |
| 255 | hsa-miR-148b-4373129 | UCAGUGCAUCACAGAACUUUGU | 33.082035 |
| 226 | hsa-miR-383-4373018 | AGAUCAGAAGGUGAUUGUGGCU | 33.127384 |
| 257 | hsa-miR-15a-4373123 | UAGCAGCACAUAAUGGUUUGUG | 33.143764 |
| 258 | hsa-miR-98-4373009 | UGAGGUAGUAAGUUGUAUUGUU | 33.154266 |
| 259 | hsa-miR-345-4373039 | UGCUGACUCCUAGUCCAGGGC | 33.263832 |
| 260 | hsa-miR-375-4373027 | UUUGUUCGUUCGGCUCGCGUGA | 33.360348 |
| 261 | hsa-miR-660-4380925 | UACCCAUUGCAUAUCGGAGUUG | 33.55976 |
| 262 | hsa-let-7f-4373164 | UGAGGUAGUAGAUUGUAUAGUU | 33.60402 |
| 263 | hsa-miR-550-4380954 | UGUCUUACUCCCUCAGGCACAU | 33.62478 |
| 252 | RNU44-4373384 | | 33.718765 |
| 252 | RNU44-4373384 | | 33.909008 |
| 264 | hsa-miR-1-4373161 | UGGAAUGUAAAGAAGUAUGUA | 33.91329 |
| 265 | hsa-miR-210-4373089 | UAGCUUAUCAGACUGAUGUUGA | 33.956635 |
| 266 | hsa-miR-189-4378067 | GUGCCUACUGAGCUGAUAUCAGU | 33.97108 |
| 252 | RNU44-4373384 | | 33.97398 |
| 253 | hsa-miR-23a-4373074 | AUCACAUUGCCAGGGAUUUCC | 34.062016 |
| 254 | hsa-let-7d-4373166 | AGAGGUAGUAGGUUGCAUAGU | 34.074215 |
| 255 | hsa-miR-424-4373201 | CAGCAGCAAUUCAUGUUUUGAA | 34.11119 |
| 252 | RNU44-4373384 | | 34.251835 |
| 256 | hsa-miR-493-4373218 | UUGUACAUGGUAGGCUUUCAUU | 34.25925 |
| 257 | hsa-miR-29c-4373289 | UAGCACCAUUUGAAAUCGGU | 34.266956 |
| 258 | hsa-miR-362-4378092 | AAUCCUUGGAACCUAGGUGUGAGU | 34.38831 |
| 259 | hsa-miR-378-4373024 | CUCCUGACUCCAGGUCCUGUGU | 34.471447 |
| 260 | hsa-miR-361-4373035 | UUAUCAGAAUCUCCAGGGGUAC | 34.52986 |
| 261 | hsa-miR-330-4373047 | GUGCAUUGUAGUUGCAUUG | 34.549976 |
| 262 | hsa-miR-145-4373133 | GUCCAGUUUUCCCAGGAAUCCCUU | 34.583946 |
| 252 | RNU44-4373384 | | 34.836792 |
| 263 | hsa-miR-107-4373154 | AGCAGCAUUGUACAGGGCUAUCA | 34.892826 |
| 264 | hsa-miR-379-4373023 | UGGUAGACUAUGGAACGUA | 35.036655 |
| 265 | hsa-miR-199a-4373272 | CCCAGUGUUCAGACUACCUGUUC | 35.053696 |
| 266 | hsa-miR-196b-4373103 | UAGGUAGUUUCCUGUUGUUGG | 35.070187 |
| 267 | hsa-miR-101-4373159 | UACAGUACUGUGAUAACUGAAG | 35.332664 |
| 268 | hsa-miR-494-4373219 | UGAAACAUACACGGGAAACCUCUU | 35.349953 |
| 269 | hsa-miR-485-5p-4373212 | AGAGGCUGGCCGUGAUGAAUUC | 35.529865 |
| 270 | hsa-miR-183-4373114 | UAUGGCACUGGUAGAAUUCACUG | 35.559986 |
| 271 | hsa-miR-326-4373050 | CCUCUGGGCCCUUCCUCCAG | 35.68104 |
| 272 | hsa-miR-572-4381017 | GUCCGCUCGGCGGUGGCCCA | 35.794235 |
| 273 | hsa-miR-657-4380922 | GGCAGGUUCUCACCCUCUCUAGG | 35.9125 |
| 274 | hsa-miR-30e-5p-4373058 | UGUAAACAUCCUUGACUGGA | 35.951595 |
| 252 | RNU44-4373384 | | 36.030655 |
| 275 | hsa-miR-7-4373014 | UGGAAGACUAGUGAUUUUGUUG | 36.180187 |
| 276 | hsa-let-7c-4373167 | UGAGGUAGUAGGUUGUAUGGUU | 34.44413 |
| 277 | hsa-miR-17-3p-4373120 | ACUGCAGUGAAGGCACUUGU | 36.803318 |
| 278 | hsa-miR-99a-4373008 | AACCCGUAGAUCCGAUCUUGUG | 37.132515 |
| 279 | hsa-miR-181b-4373116 | AACAUUCAUUGCUGUCGGUGGG | 37.506042 |
| 280 | hsa-miR-190-4373110 | UGAUAUGUUUGAUAUAUUAGGU | 38.05101 |
| 281 | hsa-miR-199a-4373100 | CCCAGUGUUUAGACUAUCUGUUC | 39.025414 |
| 282 | hsa-miR-181c-4373115 | AACAUUCAACCUGUCGGUGAGU | 39.53926 |
| | 4343438-Blank | | Not detected |
| | 4343438-Blank | | Not detected |
| 283 | hsa-let-7e-4373165 | UGAGGUAGGAGGUUGUAUAGU | Not detected |
| 284 | hsa-miR-9-4373285 | UAUUGCACUUGUCCCGGCCUG | Not detected |
| 285 | hsa-miR-9-4378074 | AAAGUGCUGUUCGUGCAGGUAG | Not detected |
| 286 | hsa-miR-10a-4373153 | UACCCUGUAGAUCCGAAUUUGUG | Not detected |
| 287 | hsa-miR-10b-4373152 | UACCCUGUAGAACCGAAUUUGU | Not detected |
| 288 | hsa-miR-18b-4373184 | UAAGGUGCAUCUAGUGCAGUUA | Not detected |
| 289 | hsa-miR-30a-3p-4373062 | CUUUCAGUCGGAUGUUUGCAGC | Not detected |
| 290 | hsa-miR-31-4373190 | GGCAAGAUGCUGGCAUAGCUG | Not detected |
| 291 | hsa-miR-33-4373048 | GCCCCUGGGCCUAUCCUAGAA | Not detected |
| 292 | hsa-miR-34a-4373278 | UGGCAGUGUCUUAGCUGGUUGUU | Not detected |
| 293 | hsa-miR-34b-4373037 | UAGGCAGUGUCAUUAGCUGAUUG | Not detected |
| 294 | hsa-miR-34c-4373036 | AGGCAGUGUAGUUAGCUGAUUGC | Not detected |
| 295 | hsa-miR-95-4373011 | UUCAACGGGUAUUUAUUGAGCA | Not detected |
| 296 | hsa-miR-96-4373010 | UUUGGCACUAGCACAUUUUUGC | Not detected |
| 297 | hsa-miR-100-4373160 | AACCCGUAGAUCCGAACUUGUG | Not detected |
| 298 | hsa-miR-105-4373157 | UCAAAUGCUCAGACUCCUGU | Not detected |
| 299 | hsa-miR-122a-4373151 | UGGAGUGUGACAAUGGUGUUUGU | Not detected |
| 300 | hsa-miR-124a-4373150 | UUAAGGCACGCGGUGAAUGCCA | Not detected |
| 301 | hsa-miR-125b-4373148 | UCCCUGAGACCCUAACUUGUGA | Not detected |
| 302 | hsa-miR-128b-4373170 | UCACAGUGAACCGGUCUCUUUC | Not detected |
| 303 | hsa-miR-129-4373171 | CUUUUUGCGGUCUGGGCUUGC | Not detected |
| 304 | hsa-miR-132-4373143 | UAACAGUCUACAGCCAUGGUCG | Not detected |
| 305 | hsa-miR-133a-4373142 | UUGGUCCCCUUCAACCAGCUGU | Not detected |
| 306 | hsa-miR-135a-4373140 | UAUGGCUUUUUAUUCCUAUGUGA | Not detected |
| 307 | hsa-miR-135b-4373139 | UAUGGCUUUUCAUUCCUAUGUG | Not detected |
| 308 | hsa-miR-137-4373174 | UAUUGCUUAAGAAUACGCGUAG | Not detected |
| 309 | hsa-miR-139-4373176 | UCUACAGUGCACGUGUCU | Not detected |
| 310 | hsa-miR-141-4373137 | UAACACUGUCUGGUAAAGAUGG | Not detected |
| 311 | hsa-miR-143-4373134 | UGAGAUGAAGCACUGUAGCUCA | Not detected |
| 312 | hsa-miR-147-4373131 | GUGUGUGGAAAUGCUUCUGC | Not detected |
| 313 | hsa-miR-149-4373128 | UCUGGCUCCGUGUCUUCACUCC | Not detected |
| 314 | hsa-miR-153-4373125 | UUGCAUAGUCACAAAAGUGA | Not detected |
| 315 | hsa-miR-182-4373271 | UUUGGCAAUGGUAGAACUCACA | Not detected |
| 316 | hsa-miR-182-4378066 | UGGUUCUAGACUUGCCAACUA | Not detected |
| 317 | hsa-miR-184-4373113 | UGGACGGAGAACUGAUAAGGGU | Not detected |
| 318 | hsa-miR-185-4373181 | UGGAGAGAAAGGCAGUUC | Not detected |
| 319 | hsa-miR-187-4373111 | UCGUGUCUUGUGUUGCAGCCG | Not detected |
| 320 | hsa-miR-193a-4373107 | AACUGGCCUACAAAGUCCCAG | Not detected |
| 321 | hsa-miR-193b-4373185 | AACUGGCCCUCAAAGUCCCGCUUU | Not detected |
| 322 | hsa-miR-194-4373106 | UGUAACAGCAACUCCAUGUGGA | Not detected |
| 323 | hsa-miR-196a-4373104 | UAGGUAGUUUCAUGUUGUUGG | Not detected |
| 324 | hsa-miR-198-4373101 | GGUCCAGAGGGGAGAUAGG | Not detected |
| 325 | hsa-miR-200a-4373273 | UAACACUGUCUGGUAACGAUGU | Not detected |
| 326 | hsa-miR-200a-4378069 | CAUCUUACCGGACAGUGCUGGA | Not detected |
| 327 | hsa-miR-200b-4381028 | UAAUACUGCCUGGUAAUGAUGAC | Not detected |
| 328 | hsa-miR-202-4373274 | AGAGGUAUAGGGCAUGGGAAAA | Not detected |
| 329 | hsa-miR-202-4378075 | UUUCCUAUGCAUAUACUUCUUU | Not detected |
| 330 | hsa-miR-203-4373095 | GUGAAAUGUUUAGGACCACUAG | Not detected |
| 331 | hsa-miR-204-4373094 | UUCCCUUUGUCAUCCUAUGCCU | Not detected |
| 332 | hsa-miR-205-4373093 | UCCUUCAUUCCACCGGAGUCUG | Not detected |
| 333 | hsa-miR-206-4373092 | UGGAAUGUAAGGAAGUGUGUGG | Not detected |
| 334 | hsa-miR-208-4373091 | AUAAGACGAGCAAAAAGCUUGU | Not detected |
| 335 | hsa-miR-211-4373088 | CUGUGCGUGUGACAGCGGCUGA | Not detected |
| 336 | hsa-miR-213-4373086 | UUCCCUUUGUCAUCCUUCGCCU | Not detected |
| 337 | hsa-miR-214-4373085 | ACAGCAGGCACAGACAGGCAG | Not detected |
| 338 | hsa-miR-215-4373084 | AUGACCUAUGAAUUGACAGAC | Not detected |
| 339 | hsa-miR-216-4373083 | UAAUCUCAGCUGGCAACUGUG | Not detected |
| 340 | hsa-miR-217-4373082 | UACUGCAUCAGGAACUGAUUGGAU | Not detected |
| 341 | hsa-miR-218-4373081 | UUGUGCUUGAUCUAACCAUGU | Not detected |
| 342 | hsa-miR-219-4373080 | UGAUUGUCCAAACGCAAUUCU | Not detected |
| 343 | hsa-miR-220-4373078 | AAGCUGCCAGUUGAAGAACUGU | Not detected |
| 344 | hsa-miR-296-4373066 | AGGGCCCCCCCUCAAUCCUGU | Not detected |
| 345 | hsa-miR-299-3p-4373189 | UAUGUGGGAUGGUAAACCGCUU | Not detected |
| 346 | hsa-miR-299-5p-4373188 | UGGUUUACCGUCCCACAUACAU | Not detected |
| 347 | hsa-miR-302a-4373275 | UAAGUGCUUCCAUGUUUUGGUGA | Not detected |
| 348 | hsa-miR-302a-4378070 | UAAACGUGGAUGUACUUGCUUU | Not detected |
| 349 | hsa-miR-302b-4373276 | UAAGUGCUUCCAUGUUUUAGUAG | Not detected |
| 350 | hsa-miR-302b-4378071 | ACUUUAACAUGGAAGUGCUUUCU | Not detected |
| 351 | hsa-miR-302c-4373277 | UAAGUGCUUCCAUGUUUCAGUGG | Not detected |
| 352 | hsa-miR-320c-4378072 | UUUAACAUGGGGGUACCUGCUG | Not detected |
| 353 | hsa-miR-302d-4373063 | UAAGUGCUUCCAUGUUUGAGUGU | Not detected |
| 354 | hsa-miR-323-4373054 | AAAAGCUGGGUUGAGAGGGCGAA | Not detected |
| 355 | hsa-miR-324-5P-4373052 | CGCAUCCCCUAGGGCAUUGGUGU | Not detected |
| 356 | hsa-miR-325-4373051 | CCUAGUAGGUGUCCAGUAAGUGU | Not detected |
| 357 | hsa-miR-329-4373191 | AACACACCUGGUUAACCUCUUU | Not detected |
| 358 | hsa-miR-337-4373044 | UCCAGCUCCUAUAUGAUGCCUUU | Not detected |
| 359 | hsa-miR-338-4373043 | UCCAGCAUCAGUGAUUUUGUUGA | Not detected |
| 360 | hsa-miR-363-4380917 | CGGGUGGAUCACGAUGCAAUUU | Not detected |
| 361 | hsa-miR-365-4373194 | UAAUGCCCCUAAAAAUCCUUAU | Not detected |
| 362 | hsa-miR-367-4373034 | AAUUGCACUUUAGCAAUGGUGA | Not detected |
| 363 | hsa-miR-368-4373033 | ACAUAGAGGAAAUUCCACGUUU | Not detected |
| 364 | hsa-miR-369-3p-4373032 | AAUAAUACAUGGUUGAUCUUU | Not detected |
| 365 | hsa-miR-369-5p-4373195 | AGAUCGACCGUGUUAUAUUCGC | Not detected |
| 366 | hsa-miR-371-4373030 | GUGCCGCCAUCUUUUGAGUGU | Not detected |
| 367 | hsa-miR-372-4373029 | AAAGUGCUGCGACAUUUGAGCGU | Not detected |
| 368 | hsa-miR-373-4373279 | GAAGUGCUUCGAUUUUGGGGUGU | Not detected |
| 369 | hsa-miR-373-4378073 | ACUCAAAAUGGGGGCGCUUUCC | Not detected |
| 370 | hsa-miR-376a-4378104 | GGUAGAUUCUCCUUCUAUGAG | Not detected |
| 371 | hsa-miR-376b-4373196 | AUCAUAGAGGAAAAUCCAUGUU | Not detected |
| 372 | hsa-miR-380-3p-4373022 | UAUGUAAUAUGGUCCACAUCUU | Not detected |
| 373 | hsa-miR-380-5p-4373021 | UGGUUGACCAUAGAACAUGCGC | Not detected |
| 374 | hsa-miR-381-4373020 | UAUACAAGGGCAAGCUCUCUGU | Not detected |
| 375 | hsa-miR-409-5p-4373197 | AGGUUACCCGAGCAACUUUGCA | Not detected |
| 376 | hsa-miR-412-4373199 | ACUUCACCUGGUCCACUAGCCGU | Not detected |
| 377 | hsa-miR-422a-4373200 | CUGGACUUAGGGUCAGAAGGCC | Not detected |
| 378 | hsa-miR-422b-4373016 | CUGGACUUGGAGUCAGAAGGCC | Not detected |
| 379 | hsa-miR-429-4373203 | UAAUACUGUCUGGUAAAACCGU | Not detected |
| 380 | hsa-miR-432-4378076 | CUGGAUGGCUCCUCCAUGUCU | Not detected |
| 381 | hsa-miR-448-4373206 | UUGCAUAUGUAGGAUGUCCCAU | Not detected |
| 382 | hsa-miR-449-4373207 | UGGCAGUGUAUUGUUAGCUGGU | Not detected |
| 383 | hsa-miR-449b-4381011 | AGGCAGUGUAUUGUUAGCUGGC | Not detected |
| 384 | hsa-miR-450-4373208 | UUUUUGCGAUGUGUUCCUAAUA | Not detected |
| 385 | hsa-miR-452-4373281 | UGUUUGCAGAGGAAACUGAGAC | Not detected |
| 386 | hsa-miR-452-4378077 | UCAGUCUCAUCUGCAAAGAAG | Not detected |
| 387 | hsa-miR-453-4373210 | GAGGUUGUCCGUGGUGAGUUCG | Not detected |
| 388 | hsa-miR-488-4373213 | CCCAGAUAAUGGCACUCUCAA | Not detected |
| 389 | hsa-miR-489-4373214 | AGUGACAUCACAUAUACGGCAGC | Not detected |
| 390 | hsa-miR-490-4373215 | CAACCUGGAGGACUCCAUGCUG | Not detected |
| 391 | hsa-miR-492-4373217 | AGGACCUGCGGGACAAGAUUCUU | Not detected |
| 392 | hsa-miR-496-4373221 | AUUACAUGGCCAAUCUC | Not detected |
| 393 | hsa-miR-497-4373222 | CAGCAGCACACUGUGGUUUGU | Not detected |
| 394 | hsa-miR-500-4373225 | AUGCACCUGGGCAAGGAUUCUG | Not detected |
| 395 | hsa-miR-501-4373226 | AAUCCUUUGUCCCUGGGUGAGA | Not detected |
| 396 | hsa-miR-502-4373227 | AUCCUUGCUAUCUGGGUGCUA | Not detected |
| 397 | hsa-miR-503-4373228 | UAGCAGCGGGAACAGUUCUGCAG | Not detected |
| 398 | hsa-miR-504-4373229 | AGACCCUGGUCUGCACUCUAU | Not detected |
| 399 | hsa-miR-505-4373230 | GUCAACACUUGCUGGUUUCCUC | Not detected |
| 400 | hsa-miR-506-4373231 | UAAGGCACCCUUCUGAGUAGA | Not detected |
| 401 | hsa-miR-507-4373232 | UUHUGCACCUUUUGGAGUGAA | Not detected |
| 402 | hsa-miR-508-4373233 | UGAUUGUAGCCUUUUGGAGUAGA | Not detected |
| 403 | hsa-miR-509-4373234 | UGAUUGGUACGUCUGUGGGUAGA | Not detected |
| 404 | hsa-miR-510-4373235 | UACUCAGGAGAGUGGCAAUCACA | Not detected |
| 405 | hsa-miR-511-4373236 | GUGUCUUUUGCUCUGCAGUCA | Not detected |
| 406 | hsa-miR-512-3p-4381034 | AAGUGCUGUCAUAGCUGAGGUC | Not detected |
| 407 | hsa-miR-512-5p-4373238 | CACUCAGCCUUGAGGGCACUUUC | Not detected |
| 408 | hsa-miR-513-4373239 | UUCACAGGGAGGUGUCAUUUAU | Not detected |
| 409 | hsa-miR-514-4373240 | AUUGACACUUCUGUGAGUAG | Not detected |
| 410 | hsa-miR-515-3p-4373241 | GAGUGCCUUCUUUUGGAGCGU | Not detected |
| 411 | hsa-miR-515-5p-4373242 | UUCUCCAAAAGAAAGCACUUUCUG | Not detected |
| 412 | hsa-miR-516-5p-4378099 | CAUCUGGAGGUAAGAAGCACUUU | Not detected |
| 413 | hsa-miR-517-4378078 | CCUCUAGAUGGAAGCACUGUCU | Not detected |
| 414 | hsa-miR-517a-4373243 | AUCGUGCAUCCCUUUAGAGUGUU | Not detected |
| 415 | hsa-miR-517b-4373244 | UCGUGCAUCCCUUUAGAGUGUU | Not detected |
| 416 | hsa-miR-517c-4373264 | AUCGUGCAUCCUUUUAGAGUGU | Not detected |
| 417 | hsa-miR-518a-4373186 | AAAGCGCUUCCCUUUGCUGGA | Not detected |
| 418 | hsa-miR-518b-4373246 | CAAAGCGCUCCCCUUUAGAGGU | Not detected |
| 419 | hsa-miR-518c-4373247 | CAAAGCGCUUCUCUUUAGAGUG | Not detected |
| 420 | hsa-miR-518c-4378082 | UCUCUGGAGGGAAGCACUUUCUG | Not detected |
| 421 | hsa-miR-518d-4373248 | CAAAGCGCUUCCCUUUGGAGC | Not detected |
| 422 | hsa-miR-518e-4373265 | AAAGCGCUUCCCUUCAGAGUGU | Not detected |
| 423 | hsa-miR-518f-4378083 | AAAGCGCUUCUCUUUAGAGGA | Not detected |
| 424 | hsa-miR-519b-4373250 | AAAGUGCAUCCUUUUAGAGGUUU | Not detected |
| 425 | hsa-miR-519c-4373251 | AAAGUGCAUCUUUUUAGAGGAU | Not detected |
| 426 | hsa-miR-519d-4373266 | CAAAGUGCCUCCCUUUAGAGUGU | Not detected |
| 427 | hsa-miR-519e-4373267 | AAAGUGCCUCCUUUUAGAGUGU | Not detected |
| 428 | hsa-miR-520a-4373268 | AAAGUGCUUCCCUUUGGACUGU | Not detected |
| 429 | hsa-miR-520b-4373252 | AAAGUGCUUCCUUUUAGAGGG | Not detected |
| 430 | hsa-miR-520c-4373253 | AAAGUGCUUCCUUUUAGAGGGUU | Not detected |
| 440 | hsa-miR-520d-4373254 | AAAGUGCUUCUCUUUGGUGGGUU | Not detected |
| 441 | hsa-miR-520e-4373255 | AAAGUGCUUCCUUUUUGAGGG | Not detected |
| 442 | hsa-miR-520f-4373256 | AAGUGCUUCCUUUUAGAGGGUU | Not detected |
| 443 | hsa-miR-520g-4373257 | ACAAAGUGCUUCCCUUUAGAGUGU | Not detected |
| 444 | hsa-miR-520h-4373258 | ACAAAGUGCUUCCCUUUAGAGU | Not detected |
| 445 | hsa-miR-521-4373259 | AACGCACUUCCCUUUAGAGUGU | Not detected |
| 446 | hsa-miR-522-4373245 | AAAAUGGUUCCCUUUAGAGUGUU | Not detected |
| 447 | hsa-miR-523-4373260 | AACGCGCUUCCCUAUAGAGGG | Not detected |
| 448 | hsa-miR-524-4378087 | GAAGGCGCUUCCCUUUGGAGU | Not detected |
| 449 | hsa-miR-526b-4378080 | AAAGUGCUUCCUUUUAGAGGC | Not detected |
| 450 | hsa-miR-542-5p-4378105 | UCGGGGAUCAUCAUGUCACGAG | Not detected |
| 451 | hsa-miR-544-4380919 | AUUCUGCAUUUUUAGCAAGU | Not detected |
| 452 | hsa-miR-545-4380918 | AUCAGCAAACAUUUAUUGUGUG | Not detected |
| 453 | hsa-miR-548a-4380948 | CAAAACUGGCAAUUACUUUUGC | Not detected |
| 454 | hsa-miR-548b-4380951 | CAAGAACCUCAGUUGCUUUUGU | Not detected |
| 455 | hsa-miR-548c-4380993 | CAAAAAUCUCAAUUACUUUUGC | Not detected |
| 456 | hsa-miR-548d-4381008 | CAAAAACCACAGUUUCUUUUGC | Not detected |
| 457 | hsa-miR-549-4380921 | UGACAACUAUGGAUGAGCUCU | Not detected |
| 458 | hsa-miR-551a-4380929 | GCGACCCACUCUUGGUUUCCA | Not detected |
| 489 | hsa-miR-551b-4380945 | GCGACCCAUACUUGGUUUCAG | Not detected |
| 490 | hsa-miR-552-4380930 | AACAGGUGACUGGUUAGACAA | Not detected |
| 491 | hsa-miR-553-4380931 | AAAACGGUGAGAUUUUGUUUU | Not detected |
| 492 | hsa-miR-554-4380932 | GCUAGUCCUGACUCAGCCAGU | Not detected |
| 493 | hsa-miR-555-4380933 | AGGGUAAGCUGAACCUCUGAU | Not detected |
| 494 | hsa-miR-556-4380934 | GAUGAGCUCAUUGUAAUAUG | Not detected |
| 495 | hsa-miR-558-4380936 | UGAGCUGCUGUACCAAAAU | Not detected |
| 496 | hsa-miR-562-4380939 | AAAGUAGCUGUACCAUUUGC | Not detected |
| 497 | hsa-miR-563-4380940 | AGGUUGACAUACGUUUCCC | Not detected |
| 498 | hsa-miR-564-4380941 | AGGCACGGUGUCAGCAGGC | Not detected |
| 499 | hsa-miR-565-4380942 | GGCUGGCUCGCGAUGUCUGUUU | Not detected |
| 500 | hsa-miR-566-4380943 | GGGCGCCUGUGAUCCCAAC | Not detected |
| 501 | hsa-miR-569-4380946 | AGUUAAUGAAUCCUGGAAAGU | Not detected |
| 502 | hsa-miR-570-4380947 | GAAAACAGCAAUUACCUUUGCA | Not detected |
| 503 | hsa-miR-575-4381020 | GAGCCAGUUGGACAGGAGC | Not detected |
| 504 | hsa-miR-576-4381021 | AUUCUAAUUUCUCCACGUCUUUG | Not detected |
| 505 | hsa-miR-578-4381022 | CUUCUUGUGCUCUAGGAUUGU | Not detected |
| 506 | hsa-miR-579-4381023 | AUUCAUUUGGUAUAAACCGCGAU | Not detected |
| 507 | hsa-miR-580-4381024 | UUGAGAAUGAUGAAUCAUUAGG | Not detected |
| 508 | hsa-miR-585-4381027 | UGGGCGUAUCUGUAUGCUA | Not detected |
| 509 | hsa-miR-586-4380949 | UAUGCAUUGUAUUUUUAGGUCC | Not detected |
| 510 | hsa-miR-587-4380950 | UUUCCAUAGGUGAUGAGUCAC | Not detected |
| 511 | hsa-miR-588-4380952 | UUGGCCACAAUGGGUUAGAAC | Not detected |
| 512 | hsa-miR-589-4380953 | UCAGAACAAAUGCCGGUUCCCAGA | Not detected |
| 513 | hsa-miR-591-4380955 | AGACCAUGGGUUCUCAUUGU | Not detected |
| 514 | hsa-miR-593-4380957 | AGGCACCAGCCAGGCAUUGCUCAGC | Not detected |
| 515 | hsa-miR-594-4380958 | CCCAUCUGGGGUGGCCUGUGACUUU | Not detected |
| 516 | hsa-miR-596-4380959 | AAGCCUGCCCGGCUCCUCGGG | Not detected |
| 517 | hsa-miR-597-4380960 | UGUGUCACUCGAUGACCACUGU | Not detected |
| 518 | hsa-miR-599-4380962 | GUUGUGUCAGUUUAUCAAAC | Not detected |
| 519 | hsa-miR-600-4380963 | ACUUACAGACAAGAGCCUUGCUC | Not detected |
| 520 | hsa-miR-601-4380965 | UGGUCUAGGAUUGUUGGAGGAG | Not detected |
| 521 | hsa-miR-603-4380972 | CACACACUGCAAUUACUUUUGC | Not detected |
| 522 | hsa-miR-606-4380974 | AAACUACUGAAAAUCAAAGAU | Not detected |
| 523 | hsa-miR-607-4380975 | GUUCAAAUCCAGAUCUAUAAC | Not detected |
| 524 | hsa-miR-608-4380976 | AGGGGUGGUGUUGGGACAGCUCCGU | Not detected |
| 525 | hsa-miR-609-4380978 | AGGGUGUUUCCUCUCAUCUCU | Not detected |
| 526 | hsa-miR-613-4380989 | AGGAAUGUUCCUUCUUUGCC | Not detected |
| 527 | hsa-miR-614-4380990 | GAACGCCUGUUCUUGCCAGGUGG | Not detected |
| 528 | hsa-miR-615-4380991 | UCCGAGCCUGGGUCUCCCUCU | Not detected |
| 529 | hsa-miR-616-4380992 | ACUCAAAACCCUUCAGUGACUU | Not detected |
| 530 | hsa-miR-617-4380994 | AGACUUCCCAUUUGAAGGUGGC | Not detected |
| 531 | hsa-miR-618-4380996 | AAACUCUACUUGUCCUUCUGAGU | Not detected |
| 532 | hsa-miR-622-4380961 | ACAGUCUGCUGAGGUUGGAGC | Not detected |
| 533 | hsa-miR-624-4380964 | UAGUACCAGUACCUUGUGUUCA | Not detected |
| 534 | hsa-miR-626-4380966 | AGCUGUCUGAAAAUGUCUU | Not detected |
| 535 | hsa-miR-627-4380967 | GUGAGUCUCUAAGAAAAGAGGA | Not detected |
| 536 | hsa-miR-629-4380969 | GUUCUCCCAACGUAAGCCCAGC | Not detected |
| 537 | hsa-miR-630-4380970 | AGUAUUCUGUACCAGGGAAGGU | Not detected |
| 538 | hsa-miR-631-4380971 | AGACCUGGCCCAGACCUCAGC | Not detected |
| 539 | hsa-miR-633-4380979 | CUAAUAGUAUCUACCACAAUAAA | Not detected |
| 540 | hsa-miR-639-4380987 | AUCGCUGCGGUUGCGAGCGCUGU | Not detected |
| 541 | hsa-miR-642-4380995 | GUCCCUCUCCAAAUGUGUCUUG | Not detected |
| 542 | hsa-miR-644-4380999 | AGUGUGGCUUUCUUAGAGC | Not detected |
| 543 | hsa-miR-645-4381000 | UCUAGGCUGGUACUGCUGA | Not detected |
| 544 | hsa-miR-646-4381002 | AAGCAGCUGCCUCUGAGGC | Not detected |
| 545 | hsa-miR-647-4381003 | GUGGCUGCACUCACUUCCUUC | Not detected |
| 546 | hsa-miR-649-4381005 | AAACCUGUGUUGUUCAAGAGUC | Not detected |
| 547 | hsa-miR-650-4381006 | AGGAGGCAGCGCUCUCAGGAC | Not detected |
| 548 | hsa-miR-651-4381007 | UUUAGGAUAAGCUUGACUUUUG | Not detected |
| 549 | hsa-miR-652-4380927 | AAUGGCGCCACUAGGGUUGUGCA | Not detected |
| 550 | hsa-miR-653-4381012 | UUGAAACAAUCUCUACUGAAC | Not detected |
| 551 | hsa-miR-654-4381014 | UGGUGGGCCGCAGAACAUGUGC | Not detected |
| 552 | hsa-miR-656-4380920 | AAUAUUAUACAGUCAACCUCU | Not detected |
| 553 | hsa-miR-658-4380923 | GGCGGAGGGAAGUAGGUCCGUUGGU | Not detected |
| 554 | hsa-miR-659-4380924 | CUUGGUUCAGGGAGGGUCCCCA | Not detected |
| 555 | hsa-miR-661-4381009 | UGCCUGGGUCUCUGGCCUGCGCGU | Not detected |
| 556 | hsa-miR-662-4381010 | UCCCACGUUGUGGCCCAGCAG | Not detected |
| 252 | RNU44-4373384 | | Not detected |
| 557 | RNU6B-4373381 | CGCAAGGAUGACACGCAAAUUCGUGAA | Not detected |
| | | GCGUUCCAUAUUUUU | |

### Example 15

The level of miR-135b and miR-429 microRNAs was examined in RNA isolated from individual plasma samples from women with ovarian cancer (n=54) as well as three groups of matched controls: healthy women (n=36), women with benign ovarian disease (n=42) and women undergoing surgery for non-ovarian malignant disease (n=14). Absolute quantitation was inferred using synthetic miRNA oligonucleotide dilution series run in parallel, permitting calculation of number of molecules (i.e., copies) of the miRNA of interest per uL of plasma. Data was normalized using C. elegans spiked-in controls as described earlier. Table _ below shows that the plasma levels of both miR-135b and miR-429 are substantially elevated in patients with cancer compared to all three control groups, demonstrating that these miRNAs are biomarkers of ovarian cancer.

TABLE 13

| | | Women with | Women undergoing surgery | Women with epithelial |
|---|---|---|---|---|
| | Healthy control | benign ovarian | for non-ovarian, non- | ovarian cancer, Stages I-IV |
| microRNA | women (n = 36) | disease (n = 42) | malignant disease (n = 14) | represented (n = 54) |
| miR-135b | 169 copies/uL | 147 copies/uL | 182 copies/uL | 1455 copies/uL |
| | plasma | plasma | plasma | plasma |
| miR-429 | 542 copies/uL | 619 copies/uL | 497 copies/uL | 1937 copies/uL |
| | plasma | plasma | plasma | plasma |

All publications and patent applications in this specification are indicative of the level of ordinary skill in the art to which this invention pertains. All publications and patent applications are herein incorporated by reference to the same extent as if each individual publication or patent application was specifically and individually indicated by reference.

The invention has been described with reference to various specific and preferred embodiments and techniques. However, it should be understood that many variations and modifications may be made while remaining within the spirit and scope of the invention.

1-40. (canceled)

41. A method comprising:
(a) extracting RNA from a biological sample of a subject;
(b) measuring a level of a miRNA in the biological sample;
(c) comparing said level of said miRNA to that in a control sample to calculate a difference in the miRNA level in the biological sample of the subject; and
(d) diagnosing a disease or disorder in said subject based on said difference in the miRNA level.

42. The method of claim 41, wherein the controlled sample is from said subject at an earlier time or from a healthy subject.

43. The method of claim 41, wherein said biological sample is blood or a cell-free fraction thereof, blood serum, blood plasma, urine, semen, seminal fluid, seminal plasma, prostatic fluid, pre-ejaculatory fluid (Cowper's fluid), pleural effusion, tears, saliva, sputum, sweat, biopsy, ascites, cerebrospinal fluid, amniotic fluid, lymph, marrow, cervical secretions, vaginal secretions, endometrial secretions, gastrointestinal secretions, bronchial secretions, breast secretions, ovarian cyst secretions, or fluid extracted from a tissue sample.

44. The method of claim 43, wherein the biological sample is blood.

45. The method of claim 41, wherein the biological sample is a biological fluid.

46. The method of claim 41, wherein the miRNA is converted to cDNA and is measured by contacting said cDNA with at least one nucleic acid probe.

47. The method of claim 46, wherein the cDNA is amplified in a polymerase chain reaction.

48. The method of claim 41, wherein the miRNA is measured by nucleic acid sequencing.

49. The method of claim 48, wherein the miRNA is converted to cDNA and is then isolated by cloning in a bacterial host.

50. The method of claim 49, wherein the cDNA is extracted from the bacterial host and sequenced by high throughput sequencing.

51. The method of any of claim 41 wherein the miRNA is selected from the group consisting of miR-100, miR135a, miR-135b, miR-141, miR-148a, miR-200a, miR-200b, miR-200c, miR-210, miR-222, miR-375, miR-205, and miR-429.

52. The method of claim 51, wherein the disease is a cancer.

53. The method of claim 52, wherein the cancer is of epithelial cell origin.

54. The method of claim 53, wherein the cancer is of prostate or ovarian origin.

55. A method of treating a disease or disorder in a subject, consisting of diagnosing said disease or disorder according to the method of claim 41, and administering a polynucleotide comprising a nucleotide sequence capable of hybridizing with said miRNA.

56. A method for measuring an epithelial cell cancer from RNA extracted from a biological sample of a subject, comprising:
(a) comparing a level of one or more miRNA in the biological sample of the subject to that in a control sample;
(b) diagnosing said cancer based on said comparison; and
(c) treating said cancer based on said diagnosis.

57. The method of claim 56, wherein said biological sample is blood or a fraction thereof, blood serum, blood plasma, urine, semen, seminal fluid, seminal plasma, prostatic fluid, pre-ejaculatory fluid (Cowper's fluid), pleural effusion, tears, saliva, sputum, sweat, biopsy, ascites, cerebrospinal fluid, amniotic fluid, lymph, marrow, cervical secretions, vaginal secretions, endometrial secretions, gastrointestinal secretions, bronchial secretions, breast secretions, ovarian cyst secretions, or fluid extracted from a tissue sample.

58. The method of claim 57, wherein the biological sample is blood.

59. The method of claim 56, wherein the biological sample is a biological fluid.

60. The method of claim 56, wherein the miRNA is selected from the group consisting of miR-100, miR135a, miR-135b, miR-141, miR-148a, miR-200a, miR-200b, miR-200c, miR-210, miR-222, miR-375, miR-205, and miR-429.
